(19) United States
(12) Patent Application Publication
(10) Pub. No.: US 2020/0289456 A1
(43) Pub. Date: Sep. 17, 2020

(54) METHODS FOR INHIBITING TUMORS AND DRUG RESISTANCE

(71) Applicant: SEQUOIA PHARMACEUTICALS, INC., Potomac, MD (US)

(72) Inventors: John E. ERICKSON, Potomac, MD (US); Catherine MACKEY, Potomac, MD (US); William CARLEY, Potomac, MD (US)

(21) Appl. No.: 16/706,440

(22) Filed: Dec. 6, 2019

Related U.S. Application Data
(63) Continuation of application No. 15/688,340, filed on Aug. 28, 2017, now abandoned.
Application No. 15/688,340 is a continuation of application No. PCT/EP2016/020142, filed on Feb. 29, 2016.
(60) Provisional application No. 62/126,529, filed on Feb. 28, 2015.

(51) Int. Cl. A61K 31/343; A61K 31/337; A61K 31/5377; A61K 45/06; A61K 31/427; A61K 31/4155

(52) U.S. Cl. CPC A61K 31/343; A61K 31/337; A61K 31/4155; A61K 45/06; A61K 31/427; A61K 31/5377

(57) ABSTRACT

Compositions and methods are provided for improving the treatment of tumors by inhibiting cytochrome P450 enzymes (“CYPs”) expressed by tumors. Inhibition of tumor-expressed CYPs provides improved dosing of anti-tumor drugs, inhibits development of drug resistance by tumors and provides improved patient outcomes.

Compositions and methods are provided for improving the treatment of tumors by inhibiting cytochrome P450 enzymes (“CYPs”) expressed by tumors. Inhibition of tumor-expressed CYPs provides improved dosing of anti-tumor drugs, inhibits development of drug resistance by tumors and provides improved patient outcomes.

### BACKGROUND

Chemotherapy is widely used to treat all forms of cancers, but its efficacy is often limited or curtailed completely due to drug resistance. For example, drug resistance accounts for nearly 90% of deaths in patients with metastatic breast cancer (Wang, Cancer Cell & Microenvironment 1:1-9, (2014)). A recent summary of mechanisms of drug resistance to cancer agents lists a number of proposed mechanisms: increased target expression; MLH1 hypermethylation; activation of survival pathways (for example, ERBB signaling pathways); increased expression of anti-apoptotic proteins (for example, FLIP, BCL-2 or MCL1); reduced cellular uptake; increased efflux; increased DNA repair; reduced target expression; topoisomerase I mutations; suppression of apoptosis; MDR1 overexpression; mutation or decreased expression of topoisomerase II; and decreased apoptosis due to mutation of p53 (Holohan, Nature Reviews Cancer 13:714-726, 2013). Efforts to combat the resistance to cancer agents have met with limited success, and usually have involved combinations of anticancer drugs, such as combinations of drugs with different mechanisms, or combinations with drugs that inhibit efflux transporters, such as Pgp.

New therapeutic agents specifically aimed at tumor cells while eliminating or minimizing systemic toxicity and combating or preventing drug resistance are desirable. It has unexpectedly been found that CYPs play an important role in enzyme-catalyzed inactivation of cancer agents, leading to drug resistance. Compositions and methods are provided for inhibiting this drug resistance using high potency, functionally irreversible CYP inhibitors (“ICIs”) to address this mechanism of drug resistance in cancer patients. Methods also are provided for using ICIs to (i) prevent the emergence of drug resistance in cancer patients, (ii) improve the efficacy of cancer drugs by using them in combinations with ICIs that inhibit drug degradation in cancer cells, (iii) prepare cancer patients for drug therapy by inhibiting CYP activity in cancer cells, (iv) improve clinical trial outcomes by reducing interpatient variability of drug exposure caused by varying CYP expression by cancer cells and (v) prevent induction of CYP activity in tumor cells and selection of drug-resistant cancer cells in patients. Methods of identifying patients for treatment also are provided in which cancer cells from the patient are tested for CYP expression and/or CYP activity. Patients whose cancer cells expressed elevated levels of CYP enzymes are identified and are treated with ICIs to inhibit CYP expression in those cells, thereby improving the outcome of treatment with anticancer drugs in those patients.

CYP-catalyzed activation of carcinogenic substances from endogenous and exogenous sources has also been identified is an important mechanism involved in carcinogenesis, tumor growth and metastatic disease, leading to increased patient deaths. Methods are provided for using ICIs to treat, as well as to prevent, the development of neoplastic diseases through the inhibition of CYP enzymes responsible for the activation of compounds with carcinogenic potential.

### SUMMARY

Methods of inhibiting cytochrome P450 enzymes are provided. Also provided are methods of treating tumors with inhibitors of cytochrome P450 enzymes, methods of inhibiting resistance to drugs that are metabolized by cytochrome P450 enzymes, methods of enhancing the therapeutic effect of anticancer drugs that are metabolized by cytochrome P450 enzymes, methods of decreasing the toxic effects of anticancer drugs that are metabolized to toxic by-products by cytochrome P450 enzymes, methods of increasing oral bioavailability of anticancer drugs that are metabolized by cytochrome p450 enzymes, methods of increasing the duration of action of anticancer drugs that are metabolized by cytochrome P450 enzymes, and methods of curing diseases that are caused or exacerbated by the activity of cytochrome P450 enzymes.

The methods described herein prevent cancer cells from inactivating an anticancer drug and also can inhibit progression of tumors to highly invasive forms. Other advantages of the methods include controlling the pharmacokinetic properties of anticancer drugs, controlling the rate of metabolism and/or degradation of anticancer drugs. The methods also can enhance the bioavailability and/or efficacy of drugs, including boosting the efficacy of certain drugs so that the drugs can be administered at a lower concentration or dosage thereby reducing their toxicity. By reducing drug dosage the methods permit a lowering of the overall cost associated with the treatment of disorders.

The methods provided include a method of inhibiting the growth of a tumor in a patient, comprising administering to the patient an effective amount of at least one cytochrome p450 monooxidase inhibitor and an effective amount of an anticancer drug, where the cytochrome p450 monooxidase inhibitor is an irreversible inhibitor, where the effective amount of the cytochrome p450 monooxidase inhibitor is effective to inhibit cytochrome P450 monooxidase activity in the tumor or in cells required for growth of the tumor and to substantially prevent degradation of the anticancer drug in the tumor or in the cells. The effective amount of the cytochrome p450 monooxidase inhibitor may be effective to inhibit cytochrome P450 monooxidase activity in the tumor. The effective amount of the cytochrome p450 monooxidase inhibitor may be effective to inhibit cytochrome P450 monooxidase activity in cells required for growth of the tumor, and where the cells are selected from the group consisting of cancer stem cells, stromal cells and endothelial cells. The cells may be cancer stem cells, stromal cells and/or endothelial cells.

In these methods the tumor may have become resistant to the antitumor activity of at least one anticancer drug. The anticancer drug may be selected from the group consisting of alkylating agents, vinca alkaloids, aromatase inhibitors, selective estrogen receptor modulators, topoisomerase I inhibitors, topoisomerase II inhibitors, microtubule stabilizing and disrupting agents, tubulin binding agents, tyrosine kinase inhibitors, proteosome inhibitors, mTOR inhibitors and conjugated antibodies.

In a specific embodiment the tumor or the cells required for the growth of the tumor may have been shown to express elevated levels of the cytochrome p450 monooxidase.

Also provided is a method of preventing or slowing the development of drug resistance in a tumor, comprising administering to a patient suffering from the tumor an effective amount of at least one cytochrome p450 monooxidase inhibitor, where the cytochrome p450 monooxidase inhibitor is an irreversible inhibitor, and where the effective amount of the cytochrome p450 monooxidase inhibitor is effective to inhibit cytochrome monooxidase activity in the tumor or in cells required for growth of the tumor and to substantially prevent degradation of a preselected anticancer drug in the tumor or in the cells.

Also provided is a method of improving a cancer therapy outcome in a patient being treated with at least one anticancer drug, comprising administering to the patient an effective amount of at least one cytochrome p450 monooxidase inhibitor, where the cytochrome p450 monooxidase inhibitor is an irreversible inhibitor, where the effective amount of the cytochrome p450 monooxidase inhibitor is effective to inhibit cytochrome P450 monooxidase activity in tumor cells or in cells required for growth of the tumor cells, and to substantially prevent degradation of a preselected anticancer drug in the tumor cells or in the cells required for growth of the tumor cells. The cancer therapy outcome may be improved efficacy and/or improved safety.

In the above methods the anticancer drug may be not substantially degraded by cytochrome activity in systemic circulation outside of the tumor or the cells required for the growth of the tumor.

Further provided is a method of preparing a patient for cancer therapy prior to treatment with an anticancer drug, comprising administering to the patient an effective amount of a cytochrome p450 monooxidase inhibitor, where the cytochrome p450 monooxidase inhibitor is an irreversible inhibitor, where the effective amount of the cytochrome p450 monooxidase inhibitor is effective to inhibit cytochrome P450 monooxidase activity in tumor cells in the patient or in cells in the patient required for growth of the tumor cells such that degradation of the anticancer drug in the tumor or in the cells is substantially inhibited upon subsequent administration of the anticancer drug to the patient. The cytochrome inhibitor may be administered to the patient prior to the first treatment with the anticancer drug.

Also provided is a method of improving a clinical trial of an anticancer drug, comprising administering to the patients in the trial an effective amount of a cytochrome p450 monooxidase inhibitor, either prior to or substantially contemporaneously with the anticancer drug. The improvement in the trial may be reduced interpatient variability of drug-degrading metabolic activity, improved clinical outcome, or reduced clinical trial size.

Further provided is a method of treating cancer in a patient comprising determining CYP3A4 and CYP3A5 expression levels in cancer cells in the patient and treating the patient with a cancer therapy selected based on the expression levels, where (a) if the expression levels are elevated the patient is (i) treated with at least one cytotoxic drug that is not substantially degraded by CYP activity or (ii) treated with an anticancer drug and an effective dose of a CYP inhibitor sufficient to substantially inhibit degradation of the anticancer drug by CYP activity or (b) if the expression levels are not elevated the patient is treated with an anticancer drug that may be a substrate for CYP activity.

In the methods described above the anticancer drug can be, but is not limited to, for example, cyclophosphamide, Ifosfamide, Vincristine, Vinblastine, Vindesine, Vinorelbine, Exemestane, Letrozole, Tamoxifen, Toremifene, Camptothecin and Camptothecan analogs such as Topotecan and Irinotecan, Etoposide, Teniposide, Taxol and Taxol analogs such as Taxotere, Erlotinib, Lapatanib, Sunitinib, Pazopanib, Imatinib, Dasatanib, Nilotinib, Bortezomib, Ibrutinib, semaxinib, vatalinib, sorafenib, leflunomide, canertinib, Temsirolimus, Cyclosporine, Tacrolimus (FK506), Sirolimus (rapamycin), Indinavir, Ritonavir, Saquinavir, Felodipine, Isradipine, Nicardipine, Nisoldipine, Nimodipine, Nitrendipine, Nifedipine, Verapamil, Etoposide, Tamoxifen, Vinblastine, Vincristine, Taxol, Atorvastatin, Fluvastatin, Lovastatin, Pravastatin, Simvastatin, Terfenadine, Loratadine, Astemizole, Alfentanil, Carbamazepine, Azithromycin, Clarithromycin, Erythromycin, Itraconazole, Rifabutin, Lidocaine, Cisapride, Sertraline, Pimozide, Triazolam, Anastrazole, Busulfan, Corticosteroids (dexamethasone, methylprednisone and prednisone), Cyclophosphamide, Cytarabine, Docetaxel, Doxorubicin, Erlotinib, Exemestane, Gefitinib, Idarubicin, Ifosphamide, Imatinib mesylate, Irinotecan, Ketoconazole, Letrozole, Paclitaxel, Teniposide, Tretinoin, Vinorelbine, telithromycin: quinidine; alprazolam, diazepam, midazolam, nelfinavir, chlorpheniramine, amlodipine, diltiazem, lercanidipine, cerivastatin, estradiol, hydrocortisone, progesterone, testosterone, alfentanyl, aripiprazole, buspirone, cafergot, caffeine, cilostazol, cocaine, codeine, dapsone, dextromethorphan, docetaxel, domperidone, eplerenone, fentanyl, finasteride, gleevec, haloperidol, irinotecan, Levo-Alpha Acetyl Methadol (LAAM), methadone, nateglinide, odanestron, propranolol, quinine, salmeterol, sildenafil, terfenadine, trazodone, vincristine, zaleplon, zolpidem, and ixabepilone, either individually or in suitable combinations.

The details of the methods are set forth in the accompanying treatment schemes and description. Further features, aspects, and advantages of the technology will become apparent from the description, the schemes, and the claims.

### BRIEF DESCRIPTION OF THE DRAWINGS

FIG. 1 shows exemplary potent ICI compounds. All compounds in the FIGURE have an IC₅₀ less than 100 nM for the metabolism of dibenzylfluorescein (DBF) by human liver microsomes (Xeno Tech, LLC, Lenexa, Kans.). Where tested, the compounds also have an IC₅₀ less than 100 nM for the inhibition of DBF metabolism by CYP 3A4 bactosomes.

### DETAILED DESCRIPTION

Compositions and methods are provided for improving the treatment of cancer by inhibiting cytochrome P450 enzymes (“CYPs”) expressed by cancer cells. Inhibition of tumor-expressed CYPs provides improved dosing of anti-tumor drugs, inhibits development of drug resistance by tumors and provides improved patient outcomes.

More specifically, it has unexpectedly been found that CYPs expressed in cancer cells play an important role in enzyme-catalyzed inactivation of cancer agents in those cells, leading to drug resistance. Compositions and methods are provided for reducing or eliminating this drug resistance using high potency, functionally irreversible CYP inhibitors (“ICIs”) that inhibit CYP enzymes expressed in cancer cells. Methods also are provided for using ICIs to (i) prevent the emergence of drug resistance in cancer patients, (ii) improve the efficacy of cancer drugs by using them in combinations with ICIs that inhibit drug degradation in cancer cells, (iii) prepare cancer patients for drug therapy by inhibiting CYP activity in cancer cells prior to treatment with anticancer drugs, (iv) improve clinical trial outcomes by reducing interpatient variability of drug exposure caused by varying CYP expression by cancer cells and (v) prevent induction of CYP activity in tumor cells and selection of drug-resistant cancer cells in patients. Methods of identifying patients for treatment also are provided in which cancer cells from the patient are tested for CYP expression and/or CYP activity and patients are identified who should receive treatment with ICIs to inhibit CYP expression in cancer cells.

In the context of the methods described herein a “tumor” or a “tumor cell” refers to a neoplastic cell that is malignant and that also may metastasize. Unless indicated otherwise a “cancer cell” can include a tumor cell or a cell required for growth of tumor cells, such as cancer stem cells, stromal cells and endothelial cells proximate to the tumor cells. The ICI may be used to inhibit CYP activity in tumor cells and in cells required for growth of tumor cells. The tumor cells may be cells of a solid tumor or of a liquid tumor, such as a leukemia or lymphoma cell.

The anticancer drugs used in combination with the ICIs as described herein can include targeted drugs, such as kinase inhibitors that preferentially inhibit kinase activity in tumor cells or drugs directed at tumor cells by selective cell binding, such as antibody-directed drugs or antibody conjugates, or non-targeted drugs that kill rapidly growing cells. In the context of the present disclosure a cytotoxic drug will be understood to include targeted and non-targeted drugs, unless indicated otherwise. Specific classes of anticancer drug that may be used with ICIs as described herein include, but are not limited to, alkylating agents, vinca alkaloids, aromatase inhibitors, selective estrogen receptor modulators, topoisomerase I inhibitors, topoisomerase II inhibitors, microtubule stabilizing and disrupting agents, tubulin binding agents, kinase inhibitors including tyrosine kinase inhibitors, proteasome inhibitors, mTOR inhibitors and conjugated antibodies, including antibodies conjugated with a cytotoxic moiety. A specific example of a conjugated antibody is Kadcyla (trastuzumab emtansine).

CYP-catalyzed activation of carcinogenic substances from endogenous and exogenous sources has also been identified is an important mechanism involved in carcinogenesis, tumor growth and metastatic disease leading to increased patient deaths. Methods are provided for using ICIs to treat, as well as to prevent, the development of neoplastic diseases through the inhibition of CYP enzymes responsible for the activation of compounds with carcinogenic potential.

Cytochrome Enzymes

Metabolism of exogenous compounds, or xenobiotics, constitute major detoxification and clearance mechanisms by which the body rids itself of unwanted chemical compounds, whether ingested or produced endogenously. The CYP1, CYP2 and CYP3 gene families are responsible for the majority of xenobiotic metabolism, and play a critical role in the breakdown and clearance of drugs. Xenobiotic P450 enzymes are widely distributed in the liver, intestines, lungs and other tissues (Krishna et al., Clinical Pharmacokinetics. 26:144-160, 1994). P450 enzymes catalyze the phase I reaction of drug metabolism, to generate metabolites for excretion. Estimates are that xenobiotic CYPs metabolize about 75% of all drugs, and that a single subfamily, CYP3A, accounts for about half of all xenobiotic CYP metabolism (Liu et al., Drug Metab. Rev. 39:699-721 2007; for a comprehensive review, see Guengerich, “Human cytochrome P450 enzymes” in: Ortiz de Montellano, PR., editor. Cytochrome P450: Structure, Mechanism, and Biochemistry. 3rd ed. Kluwer Academic-Plenum Press; New York: 2005. p. 377-530).

Use of ICIs in Cancer Treatment

Improved Pharmacokinetics and Reduced Interpatient Variability

Current cancer treatment guidelines contraindicate the co-administration of cancer drugs that are CYP3A (and particularly CYP3A4) substrates with agents that possess CYP3A inhibitory activity (see, for example, Sarantopoulos et al., Cancer chemotherapy and pharmacology 74:1113-1124 (2014)). Most cancer drugs that are metabolized by CYP3A are labeled with precautions concerning possible adverse drug interactions with known CYP3A inhibitors.

Contrary to this prejudice in the art, the instant disclosure describes methods of treatment using highly potent, functionally irreversible CYP3A inhibitors (“ICIs”) together with cancer drugs that are CYP3A substrates. This combination of CYP3A inhibitors with cancer drugs provides better pharmacokinetic parameters for the cancer drug, such as greater oral bioavailability through reduced first-pass metabolism, reduced clearance and higher steady-state Cₘᵢₙ in levels. This results in greater efficacy and better safety profiles for drugs with Cₘₐₓ-limiting toxicity. A lower Cₘₐₓ may be used for anticancer drugs while maintaining the area under the curve (AUC). The reduction in interpatient variability of drug exposure also provides an advantage in the reduction of clinical trial size.

Improved Treatment Efficacy

In addition to the methods for improving cancer drug pharmacokinetics described above, the instant disclosure provides improved treatment methods in which ICIs are used to reduce or eliminate CYP activity in tumor cells. It has unexpectedly been found that degradation of anticancer drugs inside cancer cells is a significant cause of drug resistance in those cells and that ICIs can be used to overcome this mode of drug resistance in cancer patients. More specifically, a patient receiving an anticancer drug regimen comprising at least one drug that is metabolized by an intracellular CYP is treated with an ICI at a dose that is effective to reduce drug-metabolizing CYP activity to a level that CYP-mediated drug metabolism is no longer a significant mechanism of drug resistance in the cancer cell. One skilled in the art will recognize that the dosing regimen of the ICI will depend on the identity of the ICI used, the level of CYP expression in the cancer cells and other well-understood pharmacokinetic parameters. It will also be understood that the duration of CYP inhibition required to achieve the desired goal of minimal or no substantial CYP-mediated drug resistance also will depend on the nature of the cancer drug that is being used—thus a drug that requires only a short duration of action will similarly require only a short period of CYP inhibition while a drug that has an extended duration of action will likely require an extended period of CYP inhibition. In addition, methods of measuring CYP activity in cells are known in the art and, accordingly, CYP activity can be determined in biopsy samples of cancer cells from a patient to guide the timing and duration of the desired CYP inhibition. Methods of measuring CYP activity in cancer cells are discussed in more detail below.

Reduced Systemic Toxicity and Improved Safety Profiles of Cancer Drugs

Inhibition of drug-metabolizing CYPs in this manner not only can improve efficacy of cancer drugs, but also can reduce the dosage of the cancer drug that is administered, because the drug is no longer being metabolized by CYPs to a significant extent. This in turn can improve the safety profile of an anticancer drug by lowering the systemic toxicity of the drug due to the lower dosage, or less frequent dosing, that is required. The skilled artisan will recognize that methods for determining drug dosing regimens are well known in the art.

Methods of Preventing Drug Resistance in Cancer Cells

Also provided are methods of preventing cancer cells from becoming resistant to cancer drugs, or reducing the extent of drug resistance, by administering an effective dose of one or more ICIs to the cancer cells. Anticancer drugs can induce expression of CYPs in cancer cells and thus either pretreatment with an ICI prior to administration of the cancer drug, or co-administration with the cancer drug, can inhibit activity of CYPs as soon as they are expressed, thereby preventing the cells from becoming drug resistant and/or reducing the extent of drug resistance. These methods are particularly useful in treatment-naïve patients where cancer cell CYP activity likely is low because CYP expression induced by anticancer drugs has not occurred. Once again, the skilled artisan will recognize that methods for determining dosing regimens for pretreatment or co-administration of ICIs so as to prevent CYP-mediated drug resistance can be devised using methods that are known in the art.

Preparation of Patients for Anticancer Drug Treatment

Similarly, ICIs can be used to prepare patients for anticancer drug therapy by pretreatment with one or more ICIs before beginning the drug therapy. In this manner all the cancer cells in a patient can be rendered equally susceptible to drug treatment by inhibition of intracellular CYP activity. In the absence of CYP inhibition, drug therapy will be most effective on cancer cells that have low levels of CYP activity, whereas cells with high CYP activity will be least susceptible to the anticancer drug, and drug treatment will therefore lead to selection of resistant cancer cells. Pretreatment with ICIs reduces CYP activity in all cancer cells to levels that are ineffective to degrade anticancer drugs, thereby ensuring maximum efficacy of the treatment. These methods can be used on treatment-naïve patients but are especially effective in patients who already have been treated with at least one anticancer drug, which may have led to inducement of CYP expression in the cancer cells. Administration of ICIs prior to either retreatment with the same anticancer drug or treatment with a new drug is effective to ensure optimal treatment outcomes in patients.

Improved Clinical Trials

It was discussed above how use of ICIs to inhibit systemic CYP activity, such as liver CYP activity, can be used to reduce variations in drug pharmacokinetics between patients and permit reductions in the size of patient population required for clinical trials. Similarly, use of ICIs can also reduce interpatient variability of drug exposure caused by varying CYP expression by cancer cells and also permit reductions in the size of patient populations for clinical trials. ICIs also can be used to prevent induction of CYP activity in tumor cells and/or selection of drug-resistant cancer cells in patients. Methods of identifying patients for treatment also are provided in which cancer cells from the patient are tested for CYP expression and/or CYP activity. Patients whose cancer cells express elevated levels of CYP enzymes are identified and are treated with ICIs to inhibit CYP expression in those cells, thereby improving the outcome of treatment with anticancer drugs in those patients.

Prevention of Activation of Carcinogens

CYP-catalyzed activation of carcinogenic substances from endogenous and exogenous sources has also been identified as an important mechanism involved in carcinogenesis, tumor growth, and metastatic disease, leading to increased patient deaths. Many known carcinogens, for example aromatic hydrocarbons such as benzene, are not directly carcinogenic but rather are oxidized by CYPs to metabolites that are mutagenic or otherwise carcinogenic. ICIs can be used to inhibit the CYP enzymes responsible for the activation of compounds with carcinogenic potential. An example of using an ICI to prevent activation of carcinogens would be in a patient accidentally exposed to a carcinogenic compound—ICI treatment could be used to prevent activation of the carcinogen while the patient is treated with other therapy to remove the carcinogen or while the carcinogen is excreted from the body by other metabolic processes.

Selection of ICIs

Suitable ICIs for use in the methods described herein have high potency and are functionally irreversible. A functionally irreversible inhibitor is one that either covalently interacts with the target enzyme, thereby essentially eliminating enzyme activity, or that binds so tightly to the enzyme that the inhibition is essentially irreversible.

As used herein a high potency ICI is one that has an IC₅₀ better than 100 nM, better than 50 nM, better than 25 nM or better than 10 nM in a standard in vitro assay. A suitable assay is a microtiter plate based, fluorometric assay that determines the concentration of a test compound that will decrease by half the maximal rate of metabolism of dibenzylfluorescein, a CYP3A4 substrate, by human liver microsomes. The assay may be run as described by Crespi et al. Anal. Biochem. 248: 188-90 (1997).

Suitable ICIs are described in, for example, U.S. Pat. Nos. 8,048,871, 8,148,374, 7,939,553, 8,952,056, 8,906,647, and 8,481,520, and US2011/0124578, the contents of each of which are hereby incorporated by reference in their entireties. Specific ICI compounds include ritonavir and cobicistat. In other specific examples, the ICI can be represented by a formula (I):

X-A-B-X′ I

where:

X is a lipophilic group containing from 1 to 12 carbon atoms optionally containing from 1 to 3 heteroatoms independently selected from the group consisting of O, S, and N,

A is selected from the group consisting of a bond, —OCON(R2)-, —S(O)ₙN(R2)-, —CON(R2)-, —COCO(NR2)-, —N(R2)CON(R2)-, —N(R2)S(O)ₙN(R2)-, N(R2)CO or —N(R2)COO—;

B is —(CG₁G₂)ₘ-, where m is 0-6 and where G₁ and G₂ are the same or different and where each G₁ and G₂ independently is selected from the group consisting of a bond, H, halo, haloalkyl, OR, optionally substituted alkyl, optionally substituted alkenyl, optionally substituted alkynyl, optionally substituted aryl, optionally substituted cycloalkyl, optionally substituted cycloalkylalkyl, optionally substituted aralkyl, optionally substituted heteroaryl, optionally substituted heteroaralkyl, and optionally substituted heterocycloalkyl where each optional substitution independently is selected from the group consisting of alkyl, halo, cyano, CF₃, OR, C₃-C₇ cycloalkyl, C₅-C₇ cycloalkenyl, R6, OR2, SR2, N(R2)₂, OR3, SR3, NR2R3, OR6, SR6, and NR2R6, and where G₁ and G₂, together with the atoms to which they are attached, optionally may form a 3-7-membered carbocyclic or heterocyclic ring containing up to three heteroatoms selected from the group consisting of N, S and O, and where the ring optionally may be substituted with up to 3 R7 moieties,

X′ is

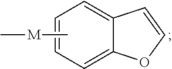

where M is selected from the group consisting of: a bond, OC(R8)_{q}, —CO—, —SOₙ—, —O—, —O—CO—, —N(D)-SOₙ—, —N(D)-COₙ—, —N(D)-(R8)_{q}-, —SOₙ—N(D)-(R8)_{q}-, or —COₙ—N(D)-(R8)_{q}-,

where M can be linked in either orientation with respect to the benzofuran ring,

where D is selected from hydrogen, alkyl, alkenyl, alkynyl, cycloalkyl, cycloalkylalkyl, heterocycloalkyl, heterocycloalkylalkyl, aryl, heteroaryl, heteroaralkyl or aralkyl, O-alkyl, where D optionally is substituted by alkyl, halo, nitro, cyano, O-alkyl, or S-alkyl;

where R is H, alkyl, haloalkyl, alkenyl, alkynyl, alkoxyalkyl, cycloalkyl, cycloalkylalkyl, heterocycloalkyl, heteroaryl, heterocycloalkylalkyl, aryl, aralkyl, and heteroaralkyl;

where each R2 is independently selected from the group consisting of H, C₁-C₁₂ alkyl, C₃-C₈ cycloalkyl, aryl, aralkyl, heteroaryl, heteroaralkyl, and heterocycloalkyl each further optionally substituted with one or more substituents selected from the group consisting of C₂-C₆ alkenyl, C₂-C₆ alkynyl, C₃-C₈ cycloalkyl, C₅-C₈ cycloalkenyl, heterocyclo; halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, ═NRPOₙN(R)₂, NRPₙOR, oxo, ═N—OR, ═N—N(R)₂, ═NR, ═NNRC(O)N(R)₂, ═NNRCOₙR, ═NNRS(O)ₙN(R)₂, and ═NNRS(O)ₙ(R);

or each R2 is independently selected from the group consisting of C₁-C₆ alkyl; substituted by aryl or heteroaryl; which groups optionally are substituted with one or more substituents selected from the group consisting of halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR;

R3 is C₂-C₆ alkenyl, C₂-C₆ alkynyl, C₃-C₈ cycloalkyl, C₅-C₈ cycloalkenyl, or heterocyclo; which groups optionally are substituted with one or more substituents selected from the group consisting of halo, OR2, R2-OH, R2-halo, NO₂, CN, COₙR2, C(O)N(R2)₂, C(O)N(R2)N(R2)₂, C(S)R2, C(S)N(R2)₂, S(O)ₙN(R2)₂, SR2, SOₙR2, N(R)₂, N(R2)COₙR2, NR2S(O)ₙR2, NR2C[═N(R2)]N(R2)₂, N(R2)N(R2)COₙR2, oxo, ═N—OR2, ═N—N(R2)₂, ═NR2, ═NNRC(O)N(R2)₂, ═NNR2C(O)ₙR2, ═NNR2S(O)ₙN(R2)₂, and ═NNR2S(O)(R2);

R6 is aryl or heteroaryl, where the aryl or heteroaryl optionally are substituted with one or more groups selected from the group consisting of aryl, heteroaryl, R2, R3, halo, OR2, R2OH, R2-halo, NO₂, CN, COₙR2, C(O)N(R2)₂, C(O)N(R2)N(R2)₂, C(S)R2, C(S)N(R2)₂, S(O)ₙN(R2)₂, SR2, SOₙR2, N(R)₂, N(R2)COₙR2, NR2S(O)ₙR2, NR2C[═N(R2)]N(R2)₂, N(R2)N(R2)COₙR2, OC(O)ₙR2, OC(S)R2, OC(O)N(R2)₂, and OC(S)N(R2)₂;

R7 is H, oxo, C₁-C₁₂ alkyl; C₃-C₈ cycloalkyl, aryl, aralkyl, heteroaryl, heteroaralkyl, or heterocycloalkyl, each further optionally substituted with one or more substituents selected from the group consisting of C₂-C₆ alkenyl, C₂-C₆ alkynyl, C₃-C₈ cycloalkyl, C₅-C₅ cycloalkenyl, heterocyclo; halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR, oxo, ═N—OR, ═N—N(R)₂, ═NR, ═NNRC(O)N(R)₂, ═NNRCOₙR, ═NNRS(O)ₙN(R)₂, and ═NNRS(O)ₙ(R);

R8 is alkyl, haloalkyl, alkenyl, alkynyl, alkoxyalkyl, cycloalkyl, cycloalkylalkyl, heterocycloalkyl, heteroaryl, heterocycloalkylalkyl, aryl, aralkyl, and heteroaralkyl;

where n=1-2, and q=0-1,

where the benzene ring of the benzofuran moiety may optionally by substituted by up to three substituents independently selected from the group consisting of R2, halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, and NRPOₙOR, where the up to three substituents do not form a ring between any adjacent carbon atoms of the benzene ring, and with the proviso that the compound does not contain a basic aliphatic amine function and does not contain a carboxylic acid group.

In a specific embodiment, the ICI may be represented by the formula II:

X-A-B-X′ II

where:

X is a lipophilic group containing from 1 to 12 carbon atoms optionally containing from 1 to 3 heteroatoms independently selected from the group consisting of O, S, and N,

A is —OCON(R2)-, —S(O)ₙN(R2)-, —CON(R2)-, —COCO(NR2)-, —N(R2)CON(R2)-, —N(R2)S(O)ₙN(R2)-, N(R2)CO or —N(R2)COO—;

B is —(CG₁G₂)ₘ-, where m is 2-6 and where G₁ and G₂ are the same or different and where each G₁ and G₂ independently is selected from the group consisting of a bond, H, halo, haloalkyl, OR, optionally substituted alkyl, optionally substituted alkenyl, optionally substituted alkynyl, optionally substituted aryl, optionally substituted cycloalkyl, optionally substituted cycloalkylalkyl, optionally substituted aralkyl, optionally substituted heteroaryl, optionally substituted heteroaralkyl, and optionally substituted heterocycloalkyl where each optional substitution independently is selected from the group consisting of alkyl, halo, cyano, CF₃, OR, C₃-C₇ cycloalkyl, C₅-C₇ cycloalkenyl, R6, OR2, SR2, N(R2)₂, OR3, SR3, NR2R3, OR6, SR6, and NR2R6, and where G₁ and G₂, together with the atoms to which they are attached, optionally may form a 3-7-membered carbocyclic or heterocyclic ring containing up to three heteroatoms selected from the group consisting of N, S and O, and where the ring optionally may be substituted with up to 3 R7 moieties,

X′ is

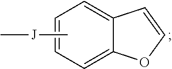

where J is selected from:

—N(D)-SOₙ—, —N(D)-COₙ—, —N(D)-(R8)_{q}-, —N(CO-D)-(R8)_{q}-, —N(SOₙ-D)-(R8)_{q}-, —SOₙ—N(D)-(R8)_{q}-, or —COₙ—N(D)-(R8)_{q}-,

where D is selected from hydrogen, alkyl, alkenyl, alkynyl, cycloalkyl, cycloalkylalkyl, heterocycloalkyl, heterocycloalkylalkyl, aryl, heteroaryl, heteroaralkyl or aralkyl, O-alkyl, O-cycloalkyl, O-cycloalkylalkyl, O-heterocycloalkyl, O-heterocycloalkylalkyl, O-heteroaralkyl O-aralkyl, N(R2)-alkyl, N(R2)-cycloalkyl, N(R2)-cycloalkylalkyl, N(R2)-heterocycloalkyl, N(R2)-heterocycloalkylalkyl, N(R2)-heteroaralkyl, N(R2)-aralkyl, wherein D optionally is substituted by alkyl, halo, nitro, cyano, O-alkyl, or S-alkyl;

where R is H, alkyl, haloalkyl, alkenyl, alkynyl, alkoxyalkyl, cycloalkyl, cycloalkylalkyl, heterocycloalkyl, heteroaryl, heterocycloalkylalkyl, aryl, aralkyl, and heteroaralkyl;

where each R2 is independently selected from the group consisting of H, C₁-C₁₂ alkyl, C₃-C₈ cycloalkyl, aryl, aralkyl, heteroaryl, heteroaralkyl, and heterocycloalkyl each further optionally substituted with one or more substituents selected from the group consisting of C₂-C₆ alkenyl, C₂-C₆ alkynyl, C₃-C₈ cycloalkyl, C₅-C₈ cycloalkenyl, heterocyclo; halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR, oxo, ═N—OR, ═N—N(R)₂, ═NR, ═NNRC(O)N(R)₂, ═NNRCOₙR, ═NNRS(O)ₙN(R)₂, and ═NNRS(O)ₙ(R);

or each R2 is independently selected from the group consisting of C₁-C₆ alkyl; substituted by aryl or heteroaryl; which groups optionally are substituted with one or more substituents selected from the group consisting of halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR;

R3 is C₂-C₆ alkenyl, C₂-C₆ alkynyl, C₃-C₈ cycloalkyl, C₅-C₈ cycloalkenyl, or heterocyclo; which groups optionally are substituted with one or more substituents selected from the group consisting of halo, OR2, R2-OH, R2-halo, NO₂, CN, COₙR2, C(O)N(R2)₂, C(O)N(R2)N(R2)₂, C(S)R2, C(S)N(R2)₂, S(O)ₙN(R2)₂, SR2, SOₙR2, N(R)₂, N(R2)COₙR2, NR2S(O)ₙR2, NR2C[═N(R2)]N(R2)₂, N(R2)N(R2)COₙR2, oxo, ═N—OR2, ═N—N(R2)₂, ═NR2, ═NNRC(O)N(R2)₂, ═NNR2C(O)ₙR2, ═NNR2S(O)ₙN(R2)₂, and ═NNR2S(O)ₙ(R2);

R6 is aryl or heteroaryl, where the aryl or heteroaryl optionally are substituted with one or more groups selected from the group consisting of aryl, heteroaryl, R2, R3, halo, OR2, R2OH, R2-halo, NO₂, CN, COₙR2, C(O)N(R2)₂, C(O)N(R2)N(R2)₂, C(S)R2, C(S)N(R2)₂, S(O)ₙN(R2)₂, SR2, SOₙR2, N(R)₂, N(R2)COₙR2, NR2S(O)ₙR2, NR2C[═N(R2)]N(R2)₂, N(R2)N(R2)COₙR2, OC(O)ₙR2, OC(S)R2, OC(O)N(R2)₂, and OC(S)N(R2)₂;

R7 is H, oxo, C₁-C₁₂ alkyl; C₃-C₈ cycloalkyl, aryl, aralkyl, heteroaryl, heteroaralkyl, or heterocycloalkyl, each further optionally substituted with one or more substituents selected from the group consisting of C₂-C₆ alkenyl, C₂-C₆ alkynyl, C₃-C₈ cycloalkyl, C₅-C₈ cycloalkenyl, heterocyclo; halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR, oxo, ═N—OR, ═N—N(R)₂, ═NR, ═NNRC(O)N(R)₂, ═NNRCOₙR, ═NNRS(O)ₙN(R)₂, and ═NNRS(O)ₙ(R);

R8 is alkyl, haloalkyl, alkenyl, alkynyl, alkoxyalkyl, cycloalkyl, cycloalkylalkyl, heterocycloalkyl, heteroaryl, heterocycloalkylalkyl, aryl, aralkyl, and heteroaralkyl;

where n=1-2, and

where q=0-1.

In another aspect, X may be alkyl, alkenyl, alkynyl, alkoxyalkyl, cycloalkyl, cycloalkylalkyl, heterocycloalkyl, heteroaryl, heterocycloalkylalkyl, aryl, aralkyl, or heteroaralkyl; where X optionally is substituted with one or more substituents selected from the group consisting of C₂-C₆ alkenyl, C₂-C₆ alkynyl, C₃-C₈ cycloalkyl, C₅-C₈ cycloalkenyl, heterocyclo; halo, OR, ROH, R-halo, NO₂, CN, COₙR, CON(R)₂, C(S)R, C(S)N(R)₂, SOₙN(R)₂, SR, SOₙR, N(R)₂, N(R)COₙR, NRS(O)ₙR, NRC[═N(R)]N(R)₂, N(R)N(R)COₙR, NRPOₙN(R)₂, NRPOₙOR, oxo, ═N—OR, ═N—N(R)₂, ═NR, ═NNRC(O)N(R)₂, ═NNRCOₙR, ═NNRS(O)ₙN(R)₂, and ═NNRS(O)ₙ(R). In one embodiment, X may be selected from the group consisting of alkyl, cycloalkyl, aryl, aralkyl, heteroaryl, and heteroaralkyl. X optionally is substituted with one or more substituents selected from the group consisting of halo, OR, ROH, R-halo, CN, COₙR, CON(R)₂, SOₙN(R)₂, SR, SOₙR, N(R)₂, N(R)COₙR, NRS(O)ₙR, oxo, and ═N—OR.

In other aspects, G₁ and G₂ may be the same or different and independently are selected from the group consisting of a bond, H, OR, optionally substituted alkyl, optionally substituted aryl, optionally substituted cycloalkyl, optionally substituted cycloalkylalkyl, optionally substituted aralkyl, optionally substituted heteroaryl, and optionally substituted heteroaralkyl. In specific embodiments, G₁ and G₂ do not form a ring, or at least one G₁ and at least one G₂ form a ring. G₁ and G₂ may be different and, in certain embodiments, neither G₁ nor G₂ is OH.

In other aspects G₁ and G₂ are selected from the group consisting of H, O-alkyl, alkyl, optionally substituted aryl and optionally substituted aralkyl.

In the embodiments above, J may be

—N(D)-SOₙ—, —N(D)-COₙ—, —N(D)-(R8)_{q}-, —N(CO-D)-(R8)_{q}-, —N(SOₙ-D)-(R8)_{q}-, —SOₙ—N(D)-(R8)_{q}-, or —COₙ—N(D)-(R8)_{q}-.

In the embodiments above, D may be hydrogen, alkyl, alkenyl, alkynyl, cycloalkyl, cycloalkylalkyl, heterocycloalkyl, heterocycloalkylalkyl, aryl, heteroaryl, heteroaralkyl or aralkyl, O-alkyl, O-cycloalkyl, O-cycloalkylalkyl, O-heterocycloalkyl, O-heterocycloalkylalkyl, O-heteroaralkyl O-aralkyl, N(R2)-alkyl, N(R2)-cycloalkyl, N(R2)-cycloalkylalkyl, N(R2)-heterocycloalkyl, N(R2)-heterocycloalkylalkyl, or N(R2)-heteroaralkyl, N(R2)-aralkyl, where D optionally is substituted by alkyl, halo, nitro, cyano, O-alkyl, or S-alkyl.

In the compounds, when X is a 5-7 membered non-aromatic monocyclic heterocycle, optionally fused or bridged with one or more 3-7 membered non-aromatic monocyclic heterocycle to form a polycyclic system, where any of the heterocyclic ring systems contains one or more heteroatoms selected from O, N, S, and P, and

when B is

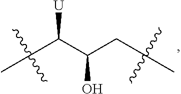

where U is selected from optionally substituted alkyl, optionally substituted alkenyl, optionally substituted alkynyl, optionally substituted aryl, optionally substituted cycloalkyl, or optionally substituted aralkyl, then J preferably is not —N(D)-SOₙ— or —N(D)-COₙ.

In other embodiments, the compound used in the methods described above does not inhibit HIV protease. In the context of the present invention, a compound is said to not inhibit HIV protease when the Ki of the compound is greater than about 1 M. Such a Ki means that the compound is not clinically useful for inhibiting HIV protease in a patient infected with HIV.

This technology also envisions the quaternization of any basic nitrogen-containing groups of the compounds disclosed herein. The basic nitrogen can be quaternized with any agents known to those of ordinary skill in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products can be obtained by such quaternization.

An exemplary ICI inhibitor has the structure (IA):

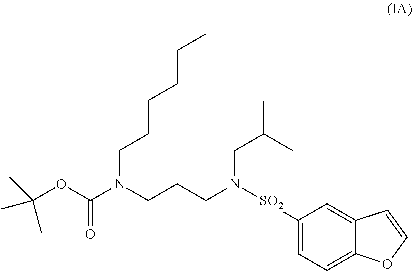 [SMILES: CCCCCCN(CCCN(CC(C)C)S(=O)(=O)c1ccc2occc2c1)C(=O)OC(C)(C)C]

In this molecule, D is isobutyl, B is —(CH₂)₃, A is —OCON(n-hexyl)-, and X is t-butyl.

The table below shows examples of various X, A, B and J moieties, although it will be recognized that these examples are merely illustrative and not limiting of ICI compounds conforming to the general structures I and II above:

| X | A | B | J |
|---|---|---|---|
| 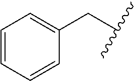 [SMILES: *Cc1ccccc1] | 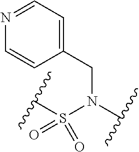 [SMILES: *N(Cc1ccncc1)S(*)(=O)=O] | 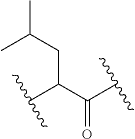 [SMILES: *C(=O)C(*)CC(C)C] | 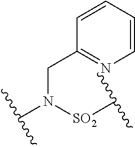 |
| 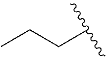 [SMILES: *CCC] | 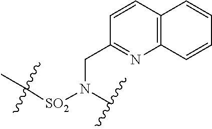 | 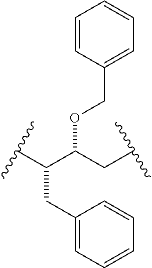 | 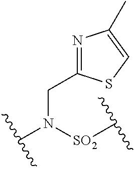 |
| 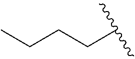 [SMILES: *CCCC] | 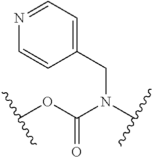 [SMILES: *OC(=O)N(*)Cc1ccncc1] | 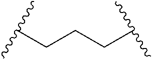 [SMILES: *CCC*] | 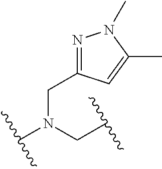 [SMILES: *CN(*)Cc1cc(C)n(C)n1] |
| 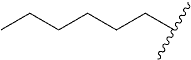 [SMILES: *CCCCCC] | 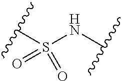 [SMILES: *NS(*)(=O)=O] | 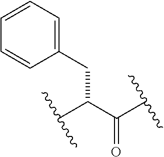 [SMILES: *C(=O)[C@H](*)Cc1ccccc1] | 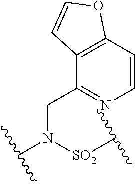 |
| 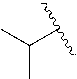 [SMILES: *C(C)C] | 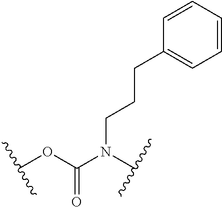 [SMILES: *OC(=O)N(*)CCCc1ccccc1] | 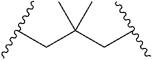 [SMILES: *CC(C)(C)C*] | 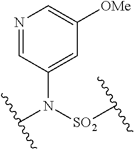 |
| 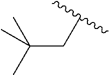 [SMILES: *CC(C)(C)C] | 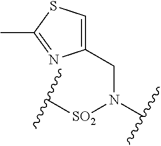 | 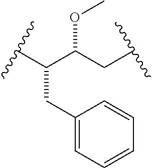 [SMILES: *C[C@@H](OC)[C@@H](*)Cc1ccccc1] | 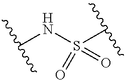 [SMILES: *NS(*)(=O)=O] |
| 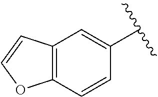 [SMILES: *c1ccc2occc2c1] | 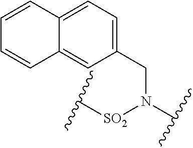 | 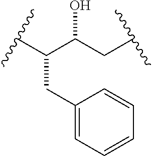 [SMILES: *C[C@@H](O)[C@@H](*)Cc1ccccc1] | 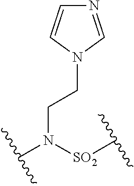 |
| 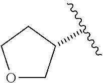 [SMILES: *[C@H]1CCOC1] | 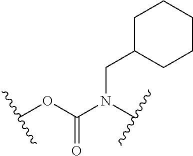 [SMILES: *OC(=O)N(*)CC1CCCCC1] | 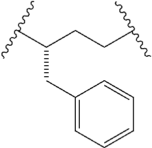 [SMILES: *CC[C@@H](*)Cc1ccccc1] | 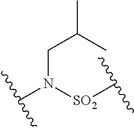 |
| 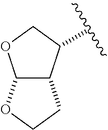 | 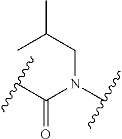 [SMILES: *C(=O)N(*)CC(C)C] | 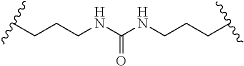 [SMILES: *CCCNC(=O)NCCC*] | 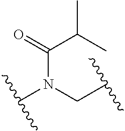 [SMILES: *CN(*)C(=O)C(C)C] |
| 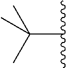 [SMILES: *C(C)(C)C] | 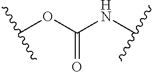 [SMILES: *NC(=O)O*] | 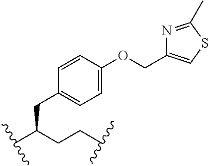 [SMILES: *CC[C@@H](*)Cc1ccc(OCc2csc(C)n2)cc1] | 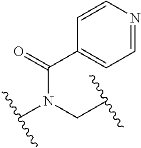 [SMILES: *CN(*)C(=O)c1ccncc1] |
| 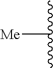 | 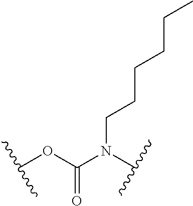 [SMILES: *OC(=O)N(*)CCCCCC] | 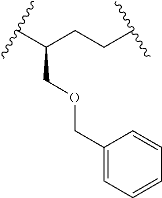 [SMILES: *CC[C@H](*)COCc1ccccc1] | 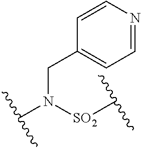 |
| | 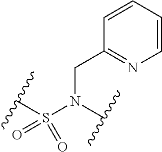 [SMILES: *N(Cc1ccccn1)S(*)(=O)=O] | 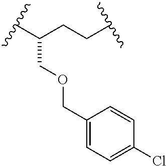 [SMILES: *CC[C@@H](*)COCc1ccc(Cl)cc1] | 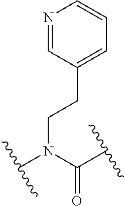 [SMILES: *C(=O)N(*)CCc1cccnc1] |
| | 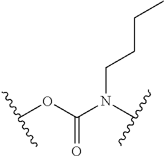 [SMILES: *OC(=O)N(*)CCCC] | | 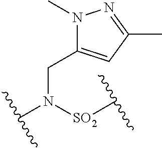 |
| | 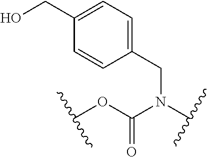 [SMILES: *OC(=O)N(*)Cc1ccc(CO)cc1] | | 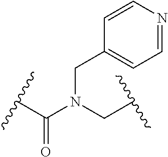 [SMILES: *CN(Cc1ccncc1)C(*)=O] |
| | 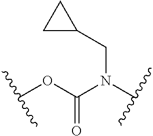 [SMILES: *OC(=O)N(*)CC1CC1] | | 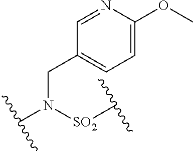 |
| | 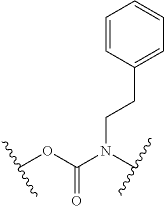 [SMILES: *OC(=O)N(*)CCc1ccccc1] | | 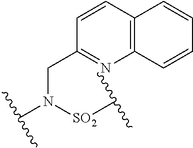 |
| | 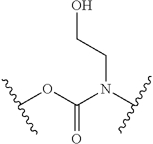 [SMILES: *OC(=O)N(*)CCO] | | 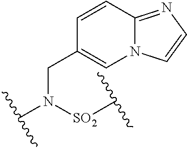 |
| | 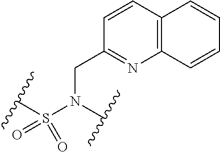 [SMILES: *N(Cc1ccc2ccccc2n1)S(*)(=O)=O] | | 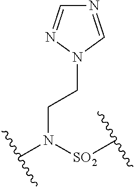 |
| | 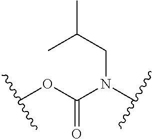 [SMILES: *OC(=O)N(*)CC(C)C] | | 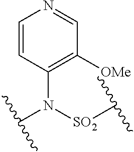 |
| | 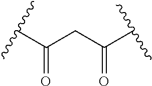 [SMILES: *C(=O)CC(*)=O] | | 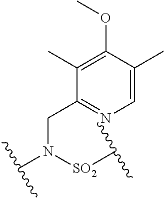 |
| | 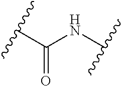 [SMILES: *NC(*)=O] | | 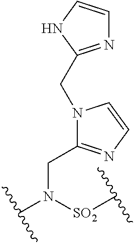 |
| | | | 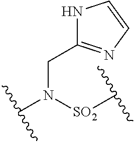 |
| | | | 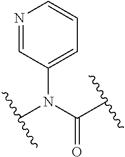 [SMILES: *C(=O)N(*)c1cccnc1] |

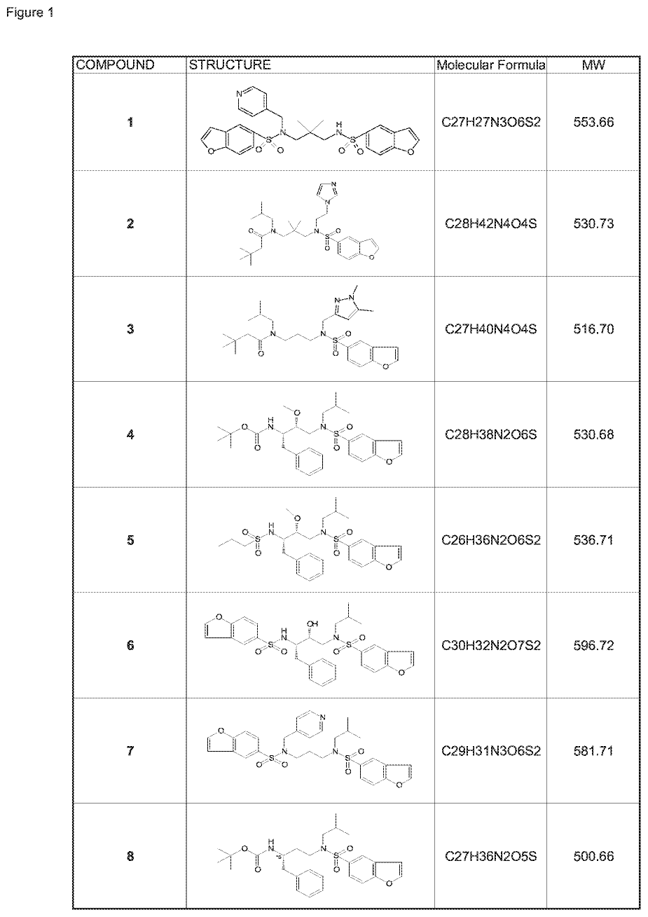

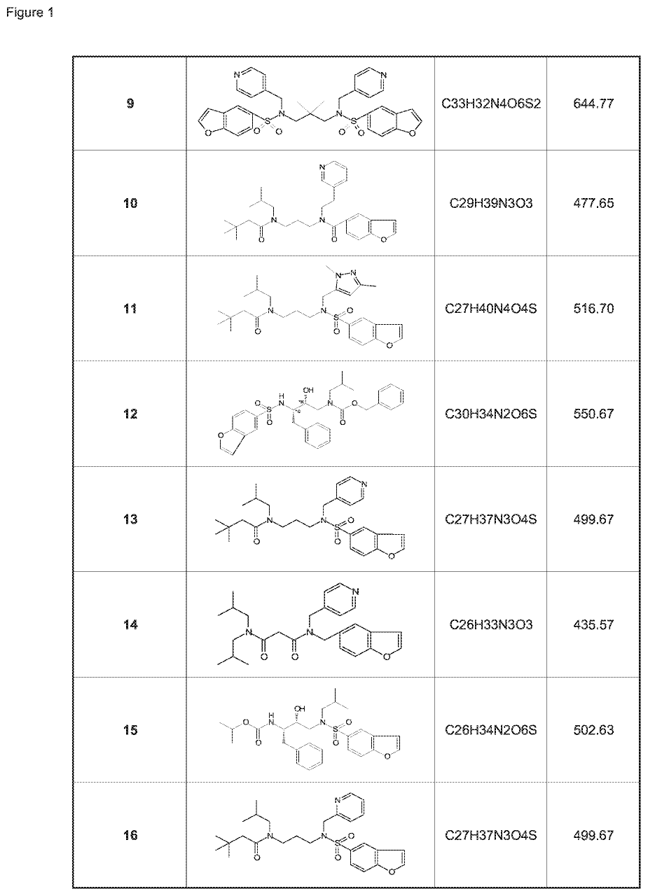

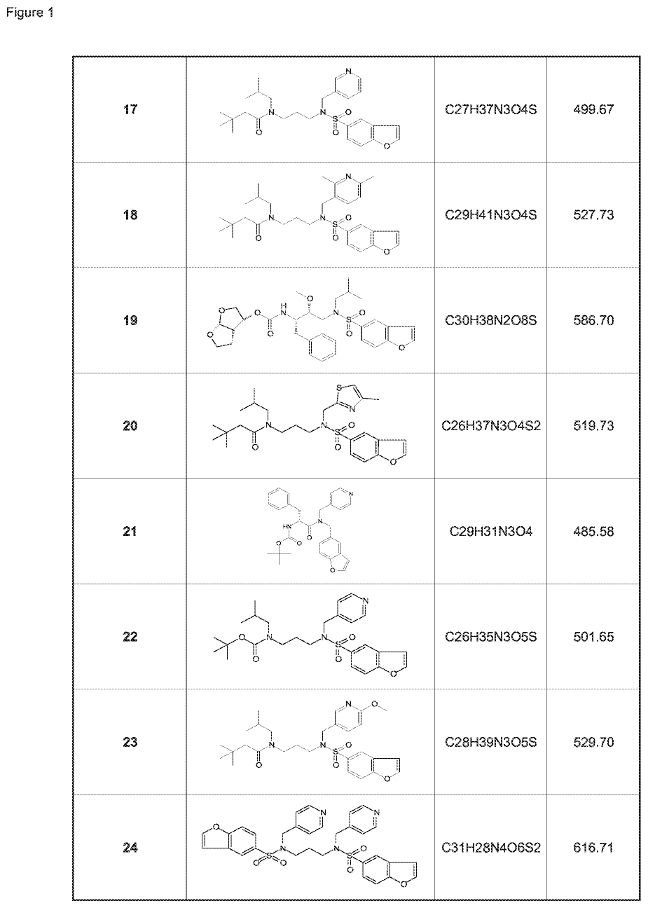

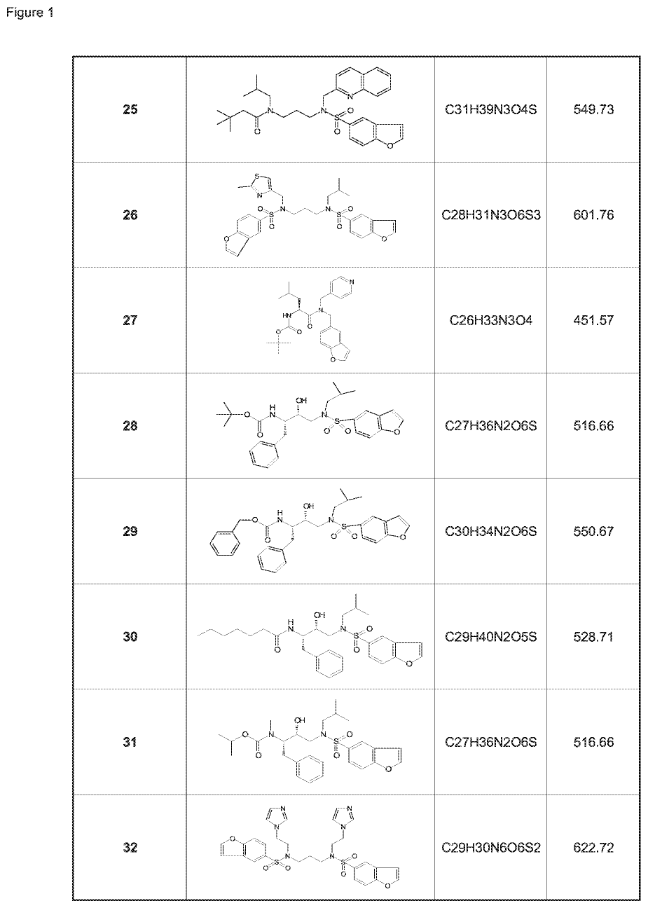

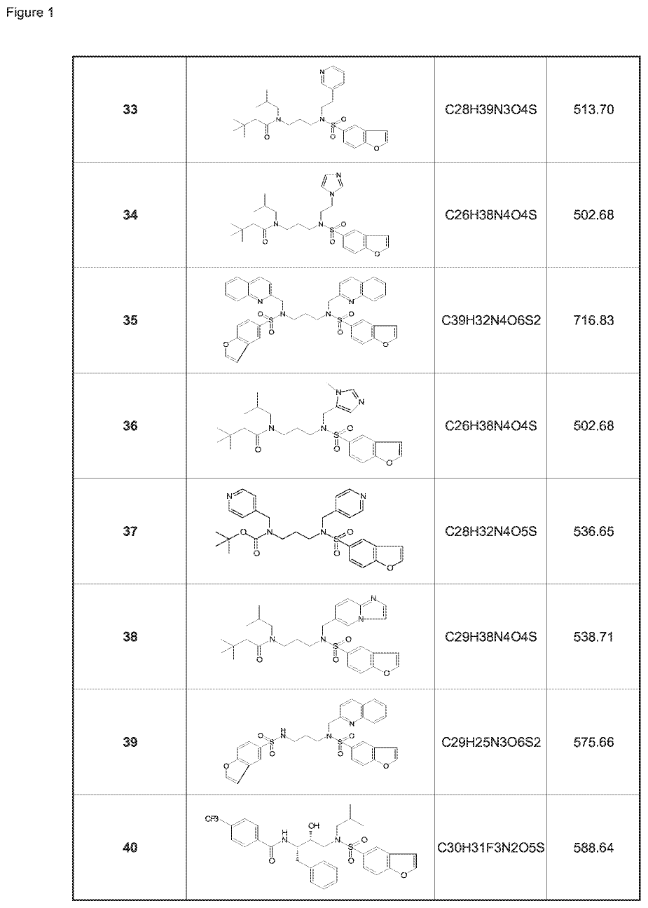

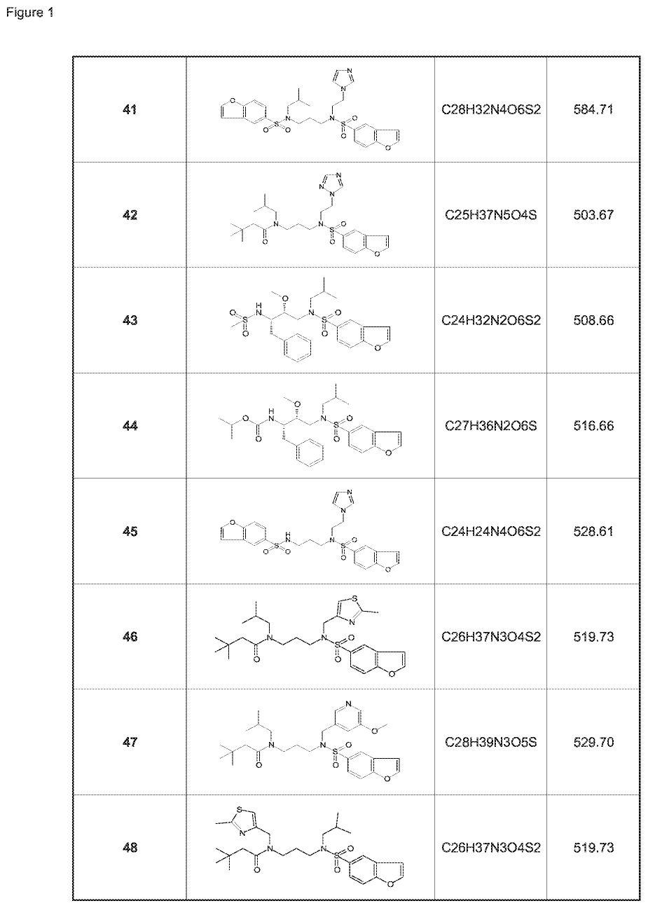

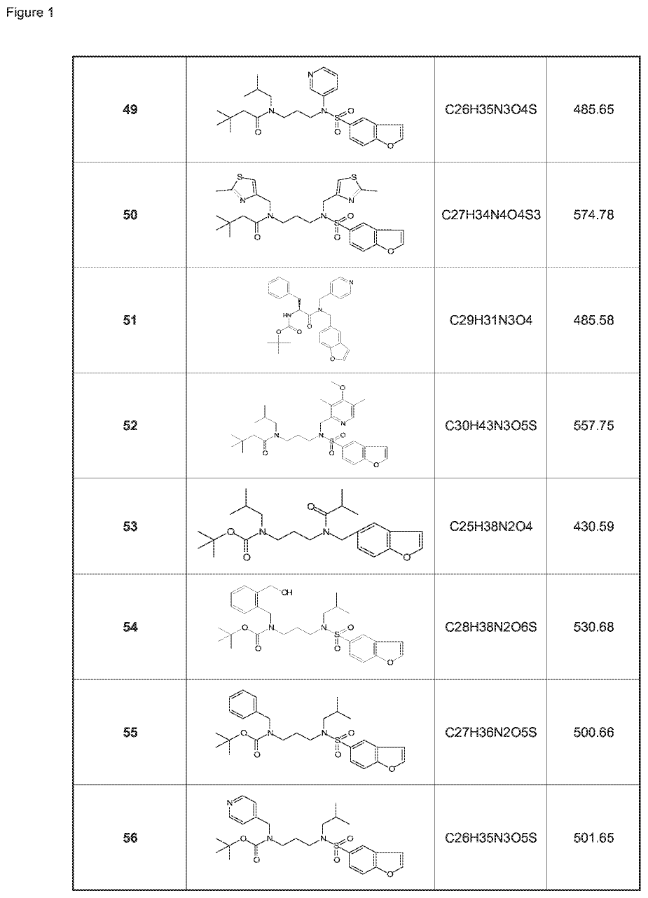

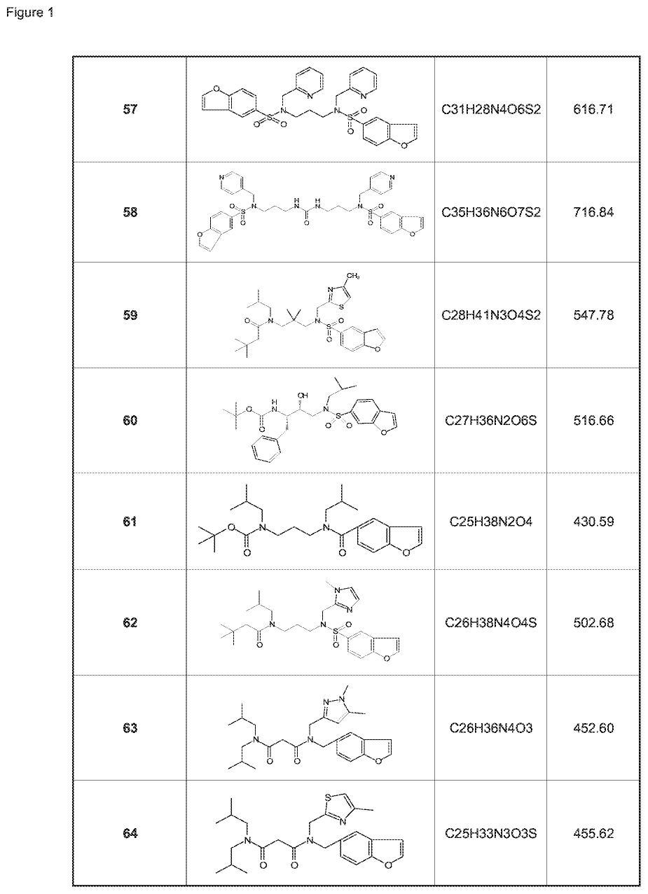

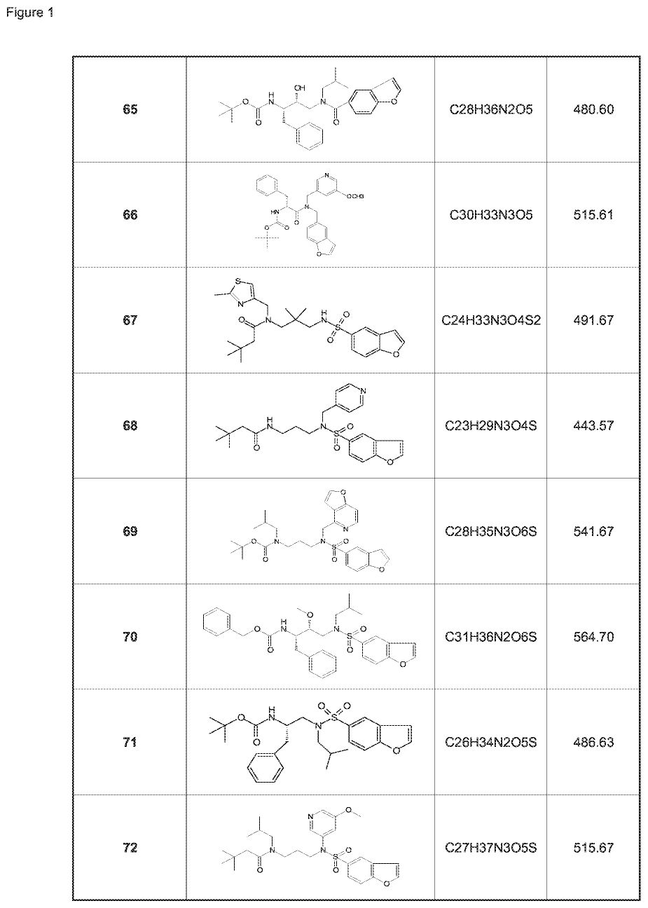

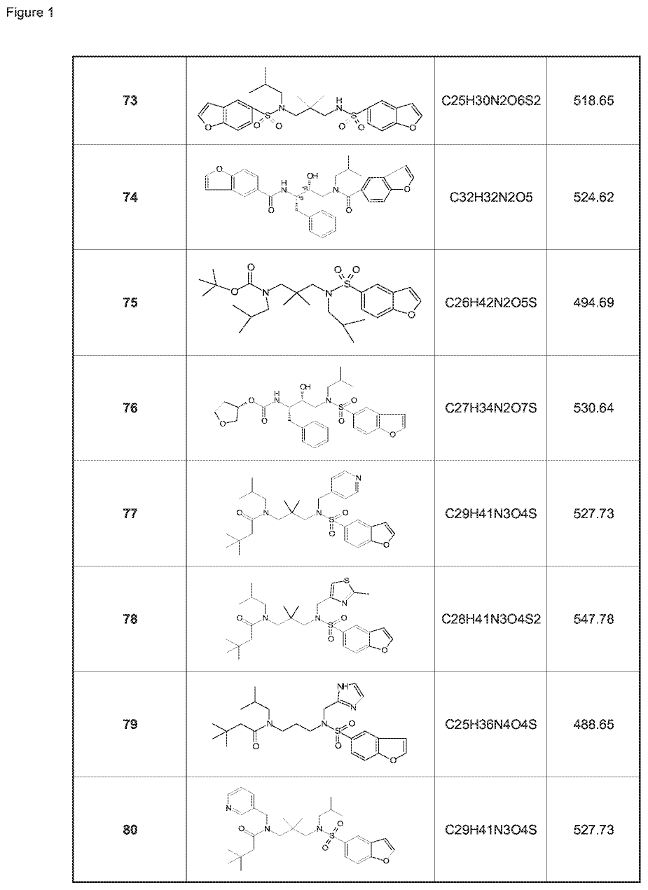

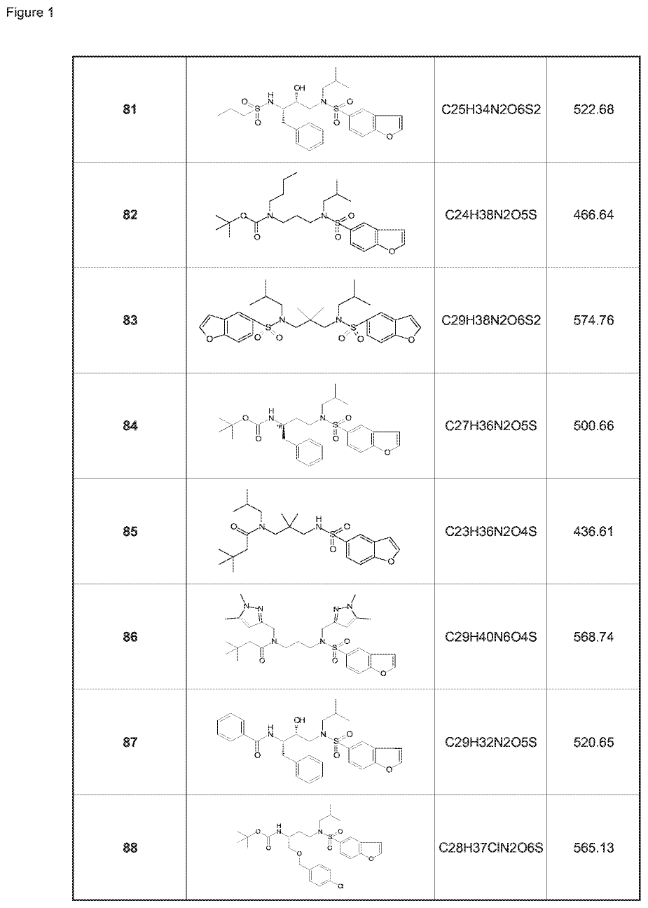

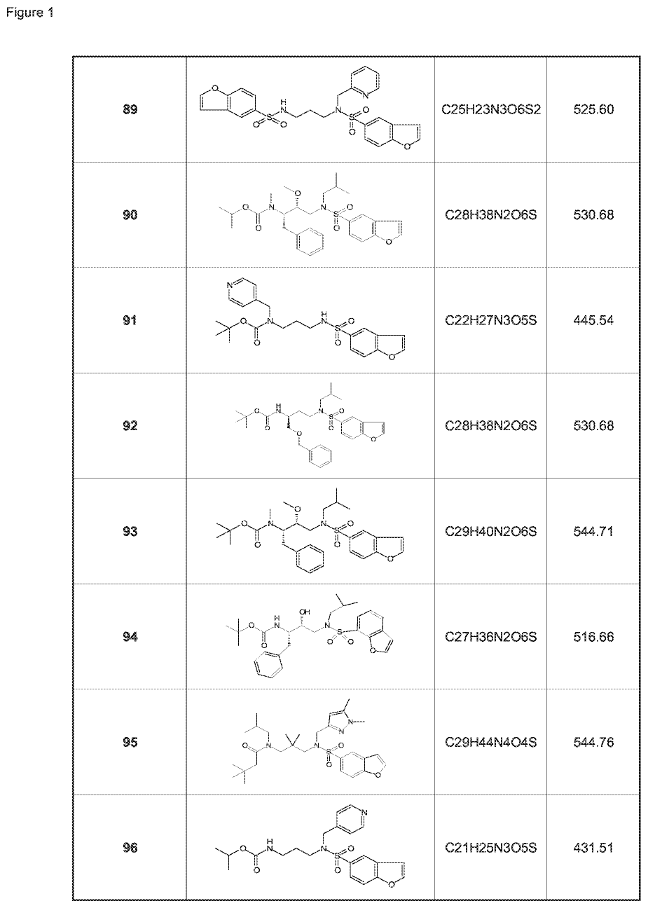

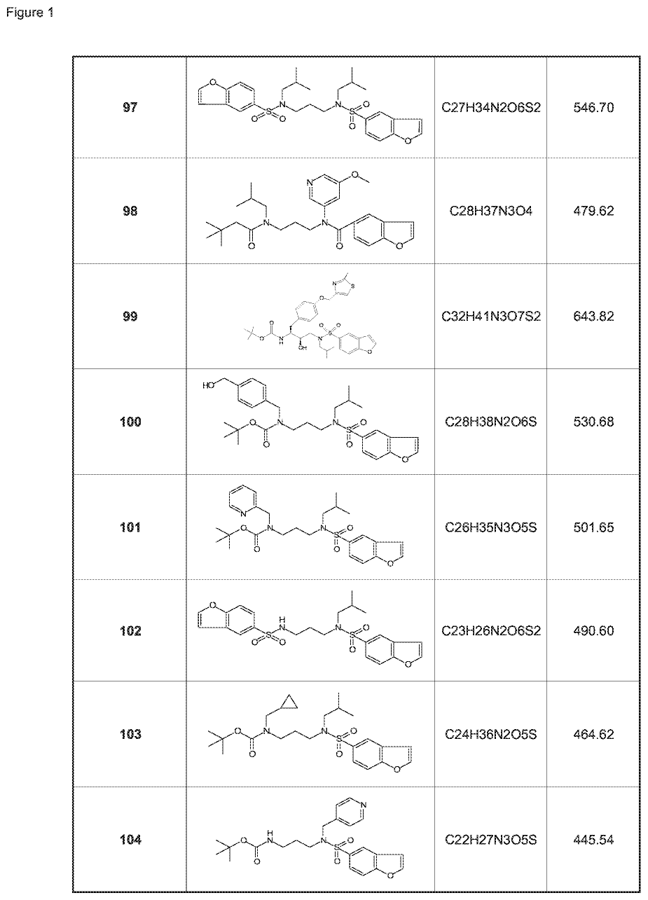

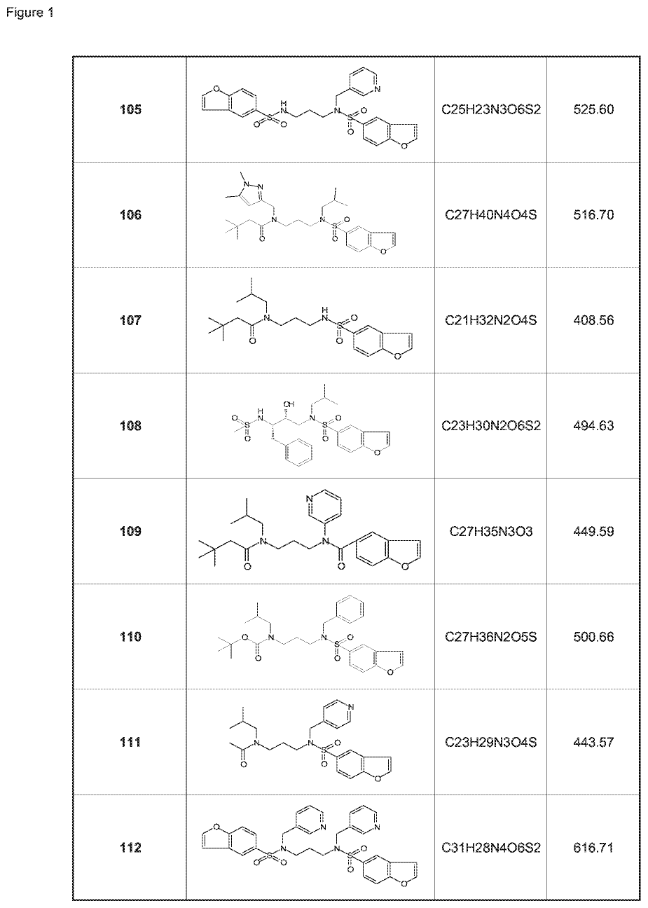

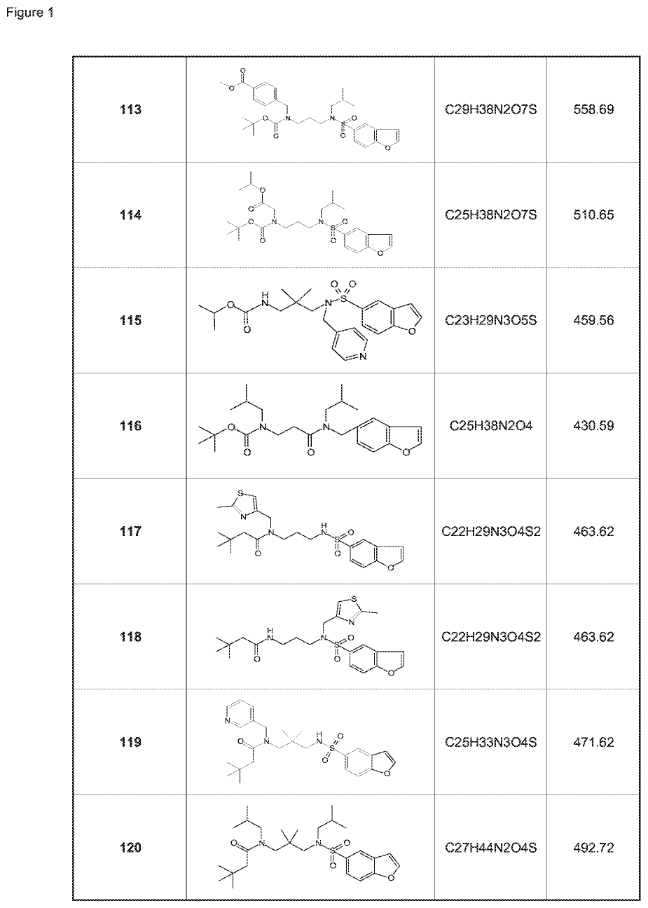

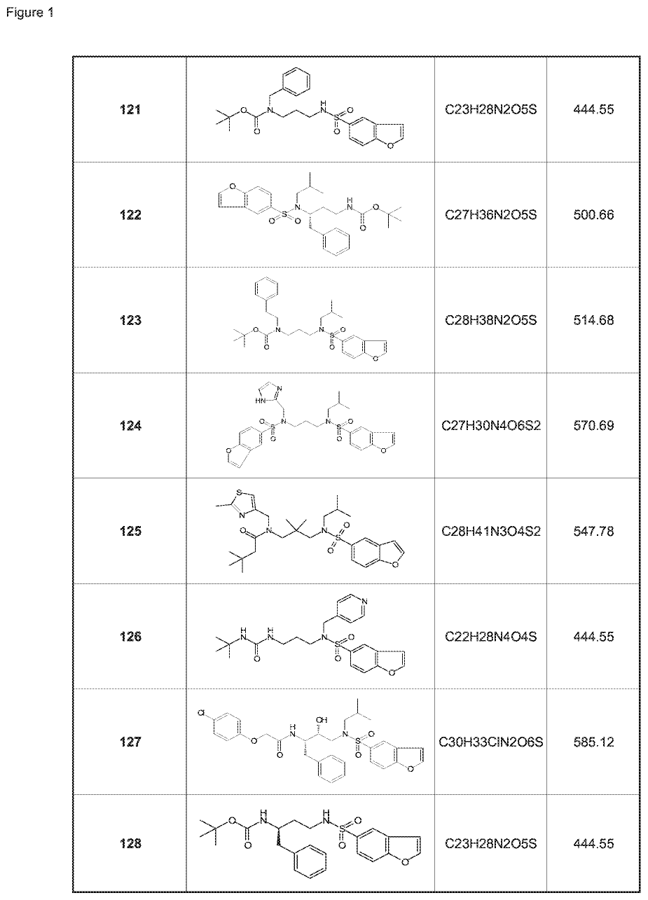

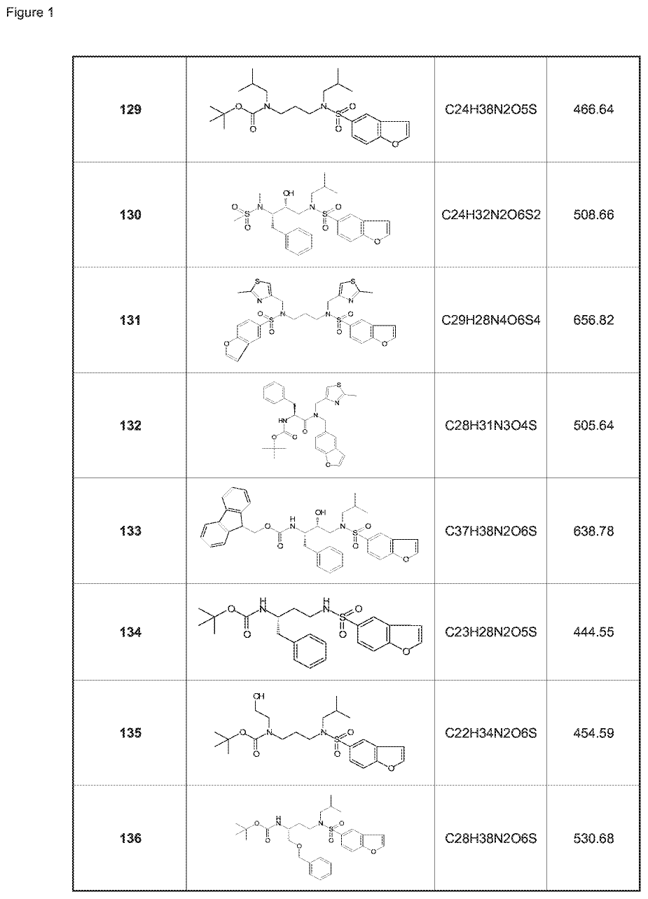

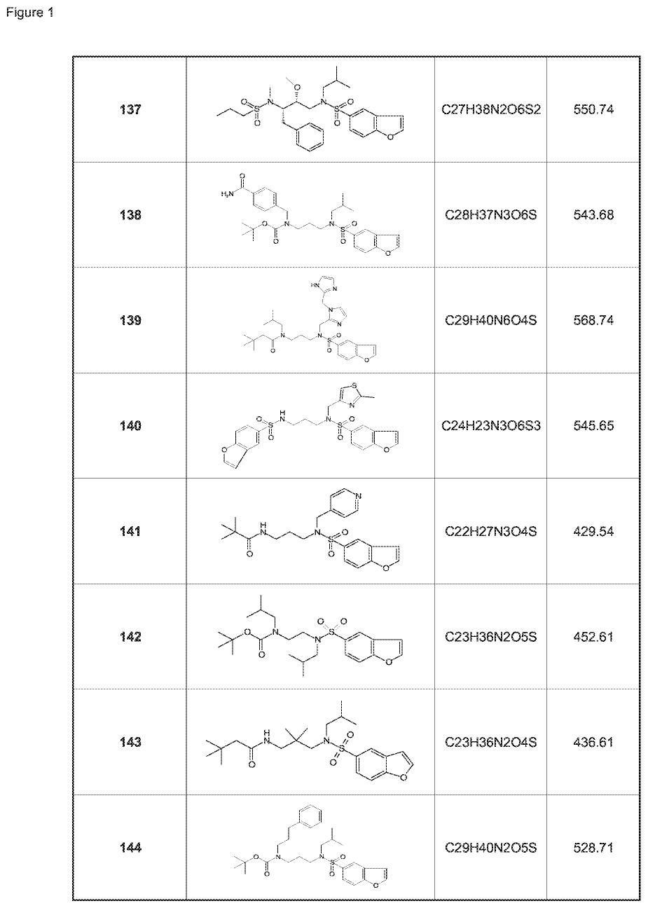

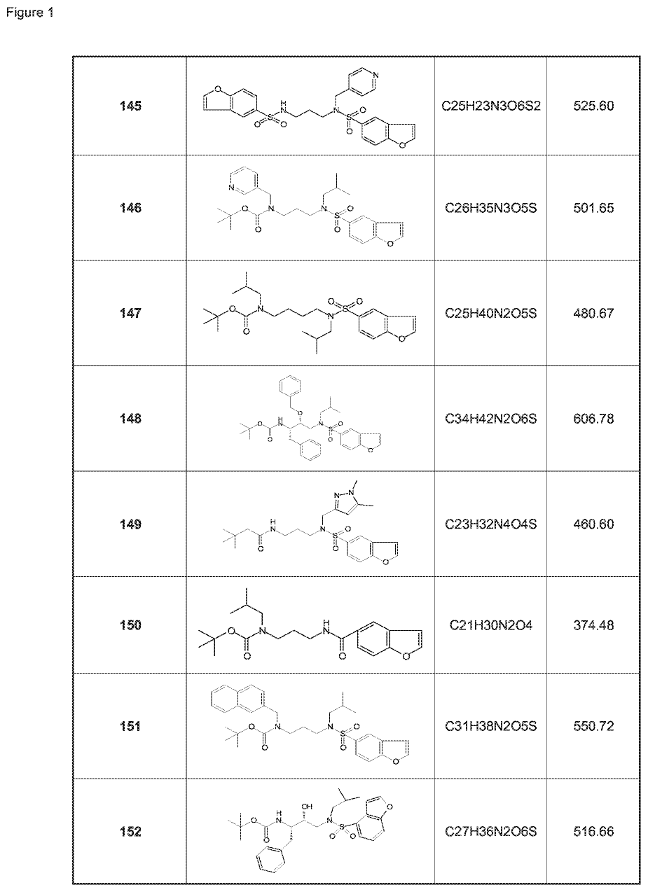

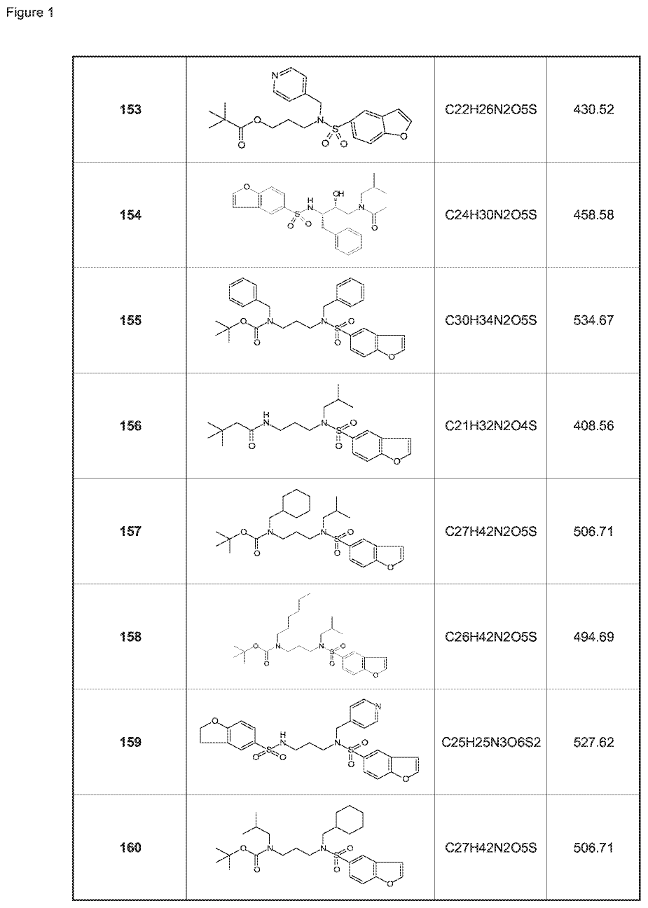

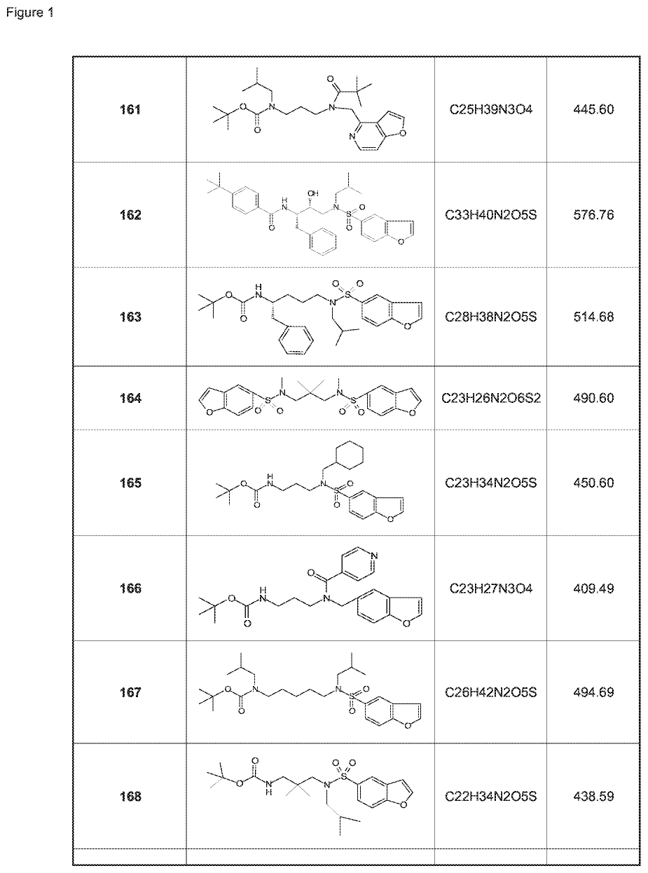

Further specific examples of ICI's conforming to formulas (I) and (II) are shown in FIG. 1.

In further examples the ICI may have the formula (III):

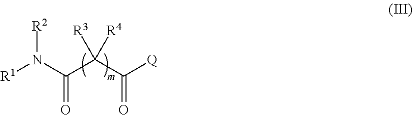

wherein:
Q is —NR⁵R⁶ or Q is —OR⁵ and R⁶ is absent;
m is 1-3;
at least one of the R¹, R², R³, R⁴, R⁵, and R⁶ groups present is C₁-C₆ alkyl substituted with an optionally substituted benzofuran;
each of R¹, R², R³, R⁴, R⁵, and R⁶ that is present is independently is selected from the group consisting of H, optionally substituted C₁-C₈ alkyl, optionally substituted C₃-C₈ cycloalkyl, optionally substituted cycloalkylalkyl, optionally substituted aryl, optionally substituted aralkyl, optionally substituted heteroaryl, optionally substituted heteroaralkyl, optionally substituted heterocyclo, optionally substituted heterocycloalkyl, and optionally substituted heterocycloalkylalkyl;
where each optional substituent is independently selected from the group consisting of halo, —CN, —NO₂, —COₙR, —CON(R)₂, —C(S)R, —C(S)N(R)₂, —SOₙN(R)₂, —SR, —SOₙR, —N(R)₂, —N(R)COₙR, —NRS(O)ₙR, —NRC[═N(R)]N(R)₂, —N(R)N(R)COₙR, —NRPOₙN(R)₂, —NRPOₙOR, oxo, ═N—OR, ═N—N(R)₂, ═NR, ═NNRC(O)N(R)₂, ═NNRCOₙR, ═NNRS(O)ₙN(R)₂, ═NNRS(O)ₙ(R)C₁-C₈ alkyl, —OR, alkyl, C₂-C₆ alkenyl, C₂-C₆ alkynyl, C₃-C₈ cycloalkyl, C₅-C₈ cycloalkenyl, heterocyclo, aryl, and heteroaryl;
each R is independently selected from the group consisting of: H, C₁-C₈ alkyl, C₃-C₈ cycloalkyl, cycloalkylalkyl, aryl, aralkyl, heteroaryl, heteroaralkyl, heterocyclo, heterocycloalkyl, and heterocycloalkylalkyl; and
each n is independently 1 or 2;
provided that at least two of the R¹, R², R³, R⁴, R⁵, and R⁶ groups present are not H; and
provided that when Q is —NR⁵R⁶, R¹ and R² are isobutyl, R³ and R⁴ are H, and R⁵ is —CH₂-[5]-benzofuranyl, then R⁶ cannot be —CH₂-4-pyridyl, —CH₂-1,5-dimethyl-3-pyrazole, or —CH2-4-methyl-2-thiazole.

In one embodiment compounds of formula (III) are compounds of formula (IV), in which Q is —NR⁵R⁶, and the compounds have the structure:

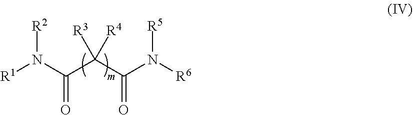

wherein:
m is 1-3;
at least one of R¹, R², R³, R⁴, R⁵, and R⁶ is C₁-C₆ alkyl substituted with an optionally substituted benzofuran;
each R¹, R², R³, R⁴, R⁵, and R⁶ independently is selected from the group consisting of H, optionally substituted C₁-C₈ alkyl, optionally substituted C₃-C₈ cycloalkyl, optionally substituted cycloalkylalkyl, optionally substituted aryl, optionally substituted aralkyl, optionally substituted heteroaryl, optionally substituted heteroaralkyl, optionally substituted heterocyclo, optionally substituted heterocycloalkyl, and optionally substituted heterocycloalkylalkyl;
where each optional substituent is independently selected from the group consisting of halo, —CN, —NO₂, —COₙR, —CON(R)₂, —C(S)R, —C(S)N(R)₂, —SOₙN(R)₂, —SR, —SOₙR, —N(R)₂, —N(R)COₙR, —NRS(O)ₙR, —NRC[═N(R)]N(R)₂, —N(R)N(R)COₙR, —NRPOₙN(R)₂, —NRPOₙOR, oxo, ═N—OR, ═N—N(R)₂, ═NR, ═NNRC(O)N(R)₂, ═NNRCOₙR, ═NNRS(O)ₙN(R)₂, ═NNRS(O)ₙ(R)C₁-C₈ alkyl, OR, C₁-C₈ alkyl, C₂-C₆ alkenyl, C₂-C₆ alkynyl, C₃-C₈ cycloalkyl, C₅-C₈ cycloalkenyl, heterocyclo, aryl, and heteroaryl;
each R is independently selected from the group consisting of: H, C₁-C₈ alkyl, C₃-C₈ cycloalkyl, cycloalkylalkyl, aryl, aralkyl, heteroaryl, heteroaralkyl, heterocyclo, heterocycloalkyl, and heterocycloalkylalkyl; and
each n is independently 1 or 2;
or a stereoisomeric form or pharmacologically acceptable salt thereof;
provided that at least two of R¹, R², R³, R⁴, R⁵, and R⁶ are not H; and provided that when R¹ and R² are isobutyl, R³ and R⁴ are H, and R⁵ is CH₂-[5]-benzofuranyl, then R⁶ cannot be —CH₂-4-pyridyl, —CH₂-1,5-dimethyl-3-pyrazolyl, or —CH2-4-methyl-2-thiazolyl.

In another embodiment, the compounds of formula (III) are compounds of formula (V), in which Q is —OR⁵, and the compounds have the structure:

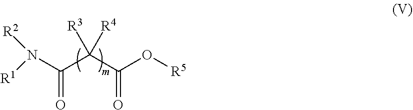

wherein:
m is 1-3;
at least one of R¹, R², R³, R⁴, and R⁵ is C₁-C₆ alkyl substituted with an optionally substituted benzofuran;
each R¹, R², R³, R⁴, and R⁵ independently is selected from the group consisting of H, optionally substituted C₁-C₈ alkyl, optionally substituted C₃-C₈ cycloalkyl, optionally substituted cycloalkylalkyl, optionally substituted aryl, optionally substituted aralkyl, optionally substituted heteroaryl, optionally substituted heteroaralkyl, optionally substituted heterocyclo, optionally substituted heterocycloalkyl, and optionally substituted heterocycloalkylalkyl;
where each optional substituent is independently selected from the group consisting of halo, —CN, —NO₂, —COₙR, —CON(R)₂, —C(S)R, —C(S)N(R)₂, —SOₙN(R)₂, —SR, —SOₙR, —N(R)₂, —N(R)COₙR, —NRS(O)ₙR, —NRC[═N(R)]N(R)₂, —N(R)N(R)COₙR, —NRPOₙN(R)₂, —NRPOₙOR, oxo, ═N—OR, ═N—N(R)₂, ═NR, ═NNRC(O)N(R)₂, ═NNRCOₙR, ═NNRS(O)ₙN(R)₂, ═NNRS(O)ₙ(R)C₁-C₈ alkyl, OR, C₁-C₈ alkyl, C₂-C₆ alkenyl, C₂-C₆ alkynyl, C₃-C₈ cycloalkyl, C₅-C₈ cycloalkenyl, heterocyclo, aryl, and heteroaryl;
each R is independently selected from the group consisting of: H, C₁-C₈ alkyl, C₃-C₈ cycloalkyl, cycloalkylalkyl, aryl, aralkyl, heteroaryl, heteroaralkyl, heterocyclo, heterocycloalkyl, and heterocycloalkylalkyl; and
each n is independently 1 or 2;
or a stereoisomeric form or pharmacologically acceptable salt thereof;
provided that at least two of R¹, R², R³, R⁴, and R⁵ are not H.

Each of the aspects and embodiments of the technology discussed above can include one or more of the following embodiments, including the following embodiments of a compound of formula (III), formula (IV), or formula (V).

In some embodiments R⁵ is C₁-C₆ alkyl substituted with an otherwise unsubstituted benzofuran, wherein said alkyl is linked to the 4, 5, 6 or 7 position of the benzofuran, for example, when m is 1,

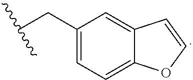 [SMILES: *Cc1ccc2occc2c1]

In other embodiments, R³ is H and R⁴ is selected from the group consisting of H, optionally substituted C₁-C₈ alkyl, optionally substituted C₃-C₈ cycloalkyl, optionally substituted cycloalkylalkyl, optionally substituted aryl, optionally substituted aralkyl, optionally substituted heteroaryl, optionally substituted heteroaralkyl, optionally substituted heterocyclo, optionally substituted heterocycloalkyl, and optionally substituted heterocycloalkylalkyl. In such an embodiment R⁵ may be C₁-C₆ alkyl substituted with an otherwise unsubstituted benzofuran, wherein said alkyl is linked to the 4, 5, 6, or 7 position of the benzofuran, e.g. R⁵ is —CH₂-5-benzofuran

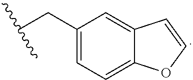 [SMILES: *Cc1ccc2occc2c1]

In some embodiments R¹ is optionally substituted C₁-C₈ alkyl.

In some embodiments R² is optionally substituted C₁-C₈ alkyl.

In some embodiments R⁴ is optionally substituted alkyl or optionally substituted heteroaralkyl.

In some embodiments, where Q is —NR⁵R⁶, R⁶ is H, optionally substituted C₁-C₈ alkyl, or optionally substituted heteroaralkyl.

In some embodiments R¹ and R² are optionally substituted C₁-C₈ alkyl.

In some embodiments where Q is —NR⁵R⁶, R³ is H, R⁴ is H, optionally substituted C₁-C₈ alkyl or optionally substituted heteroaralkyl, and R⁶ is H, optionally substituted C₁-C₈ alkyl or optionally substituted heteroaralkyl.

In some embodiments R³ is C₁-C₆ alkyl substituted with an otherwise unsubstituted benzofuran, wherein said alkyl is linked to the 4, 5, 6, or 7 position of the benzofuran. For example, when m is 1, R³ is

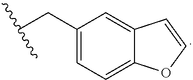 [SMILES: *Cc1ccc2occc2c1]

In other embodiments R³

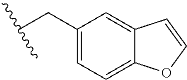 [SMILES: *Cc1ccc2occc2c1]

regardless of the value of m.

In some embodiments R⁴ is H or C₁-C₈ alkyl.

In some embodiments R⁴ is H.

In some embodiments, for example where Q is —NR⁵R⁶, R⁴ is H and R⁵ is H.

In some embodiments R⁶ is selected from the group consisting of optionally substituted C₁-C₈ alkyl, optionally substituted C₃-C₈ cycloalkyl, optionally substituted cycloalkylalkyl, optionally substituted aryl, optionally substituted aralkyl, optionally substituted heteroaryl, optionally substituted heteroaralkyl, optionally substituted heterocyclo, optionally substituted heterocycloalkyl, and optionally substituted heterocycloalkylalkyl.

In some embodiments R¹ is C₁-C₆ alkyl substituted with an otherwise unsubstituted benzofuran, wherein said alkyl is linked to the 4, 5, 6, or 7 position of the benzofuran, for example, when m is 1,

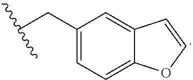 [SMILES: *Cc1ccc2occc2c1]

In some embodiments R³ and R⁴ are each independently heteroaralkyl.

In some embodiments R³ and R⁴ are each independently heteroaryl methyl.

In some embodiments the compounds are selected from the compounds listed in Table 1.

In some embodiments the cytochrome P450 monooxygenase is CYP3A4 or CYP3A5.

In some embodiments, for example when Q is —OR⁵, R⁵ is not H. In other embodiments, where Q is —OR⁵, R⁵ is C₁-C₆ alkyl substituted with benzofuran, e.g.,

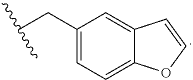 [SMILES: *Cc1ccc2occc2c1]

In other embodiments, for example where Q is —NR⁵R⁶, neither R⁵ nor R⁶ are H.

In some embodiments, compounds and compositions comprising compounds of formula III are limited to those comprising compounds of the formula (IV) or compounds of formula (V), wherein the R¹, R², R³, R⁴, R⁵ and R⁶ group present in the compounds are independently selected from those found in the following table.

| R¹ and R² | R³ and R⁴ | R⁵ and R⁶ |
|---|---|---|
| H | H | H |
| 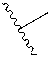 | 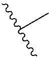 | |
| 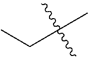 [SMILES: *CC] | | 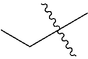 [SMILES: *CC] |
| 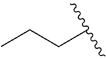 [SMILES: *CCC] | | 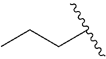 [SMILES: *CCC] |
| 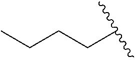 [SMILES: *CCCC] | | |
| 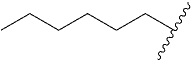 [SMILES: *CCCCCC] | | |
| 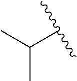 [SMILES: *C(C)C] | | |
| 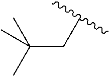 [SMILES: *CC(C)(C)C] | 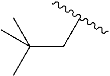 [SMILES: *CC(C)(C)C] | 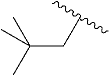 [SMILES: *CC(C)(C)C] |
| 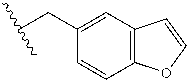 [SMILES: *Cc1ccc2occc2c1] | 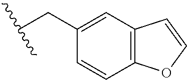 [SMILES: *Cc1ccc2occc2c1] | 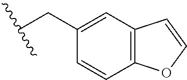 [SMILES: *Cc1ccc2occc2c1] |
| 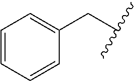 [SMILES: *Cc1ccccc1] | | |
| 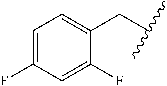 [SMILES: *Cc1ccc(F)cc1F] | | |
| 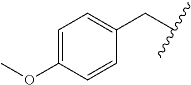 [SMILES: *Cc1ccc(OC)cc1] | 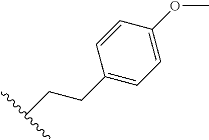 [SMILES: *CCc1ccc(OC)cc1] | 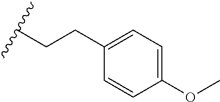 [SMILES: *CCc1ccc(OC)cc1] |
| | | 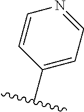 [SMILES: *c1ccncc1] |
| | 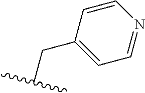 [SMILES: *Cc1ccncc1] | 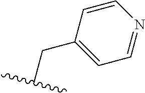 [SMILES: *Cc1ccncc1] |
| | 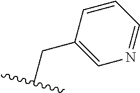 [SMILES: *Cc1cccnc1] | 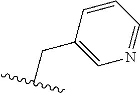 [SMILES: *Cc1cccnc1] |
| | 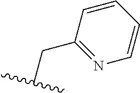 [SMILES: *Cc1ccccn1] | |
| | 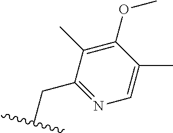 [SMILES: *Cc1ncc(C)c(OC)c1C] | |
| | 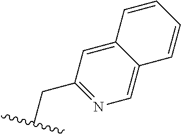 [SMILES: *Cc1cc2ccccc2cn1] | |
| | 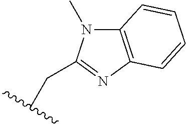 [SMILES: *Cc1nc2ccccc2n1C] | |
| | 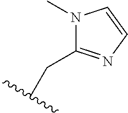 [SMILES: *Cc1nccn1C] | 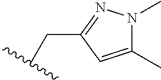 [SMILES: *Cc1cc(C)n(C)n1] |
| | 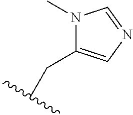 [SMILES: *Cc1cncn1C] | |
| | 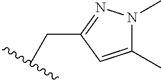 [SMILES: *Cc1cc(C)n(C)n1] | 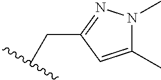 [SMILES: *Cc1cc(C)n(C)n1] |
| | 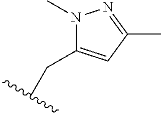 [SMILES: *Cc1cc(C)nn1C] | |
| | 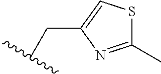 [SMILES: *Cc1csc(C)n1] | |
| | 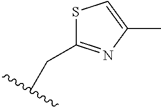 [SMILES: *Cc1nc(C)cs1] | |
| | 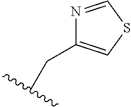 [SMILES: *Cc1cscn1] | |
| | 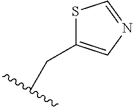 [SMILES: *Cc1cncs1] | |
| | 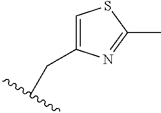 [SMILES: *Cc1csc(C)n1] | |
| 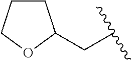 [SMILES: *CC1CCCO1] | | 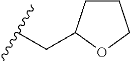 [SMILES: *CC1CCCO1] |

In some embodiments the compounds of formula (III) are selected from the compounds listed in the table below. It will be recognized that the compounds in the table are merely illustrative examples and are not limiting. All compounds in the table have an IC₅₀ less than 100 nM for the metabolism of dibenzylfluorescein (DBF) by human liver microsomes (Xeno Tech, LLC, Lenexa, Kans.). Where tested, the compounds also have an IC₅₀ less than 100 nM for the inhibition of DBF metabolism by CYP 3A4 bactosomes.

TABLE 1

| | |
|---|---|
| 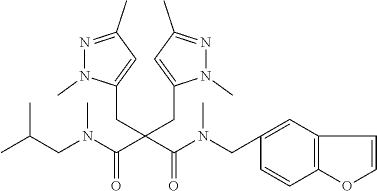 [SMILES: Cc1cc(CC(Cc2cc(C)nn2C)(C(=O)N(C)Cc2ccc3occc3c2)C(=O)N(C)CC(C)C)n(C)n1] | Cmpd. A 1 |
| 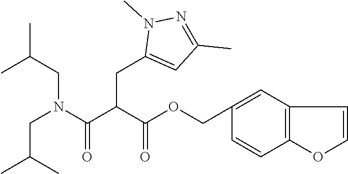 [SMILES: Cc1cc(CC(C(=O)OCc2ccc3occc3c2)C(=O)N(CC(C)C)CC(C)C)n(C)n1] | Cmpd. A 2 |
| 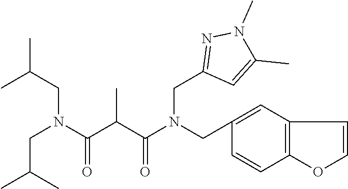 [SMILES: Cc1cc(CN(Cc2ccc3occc3c2)C(=O)C(C)C(=O)N(CC(C)C)CC(C)C)nn1C] | Cmpd. A 3 |
| 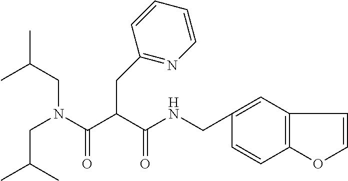 [SMILES: CC(C)CN(CC(C)C)C(=O)C(Cc1ccccn1)C(=O)NCc1ccc2occc2c1] | Cmpd. A 4 |
| 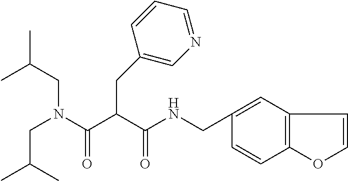 [SMILES: CC(C)CN(CC(C)C)C(=O)C(Cc1cccnc1)C(=O)NCc1ccc2occc2c1] | Cmpd. A 5 |
| 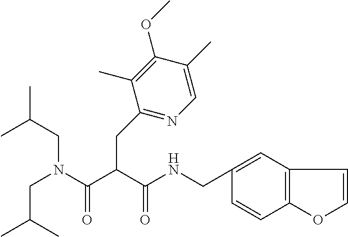 [SMILES: COc1c(C)cnc(CC(C(=O)NCc2ccc3occc3c2)C(=O)N(CC(C)C)CC(C)C)c1C] | Cmpd. A 6 |
| 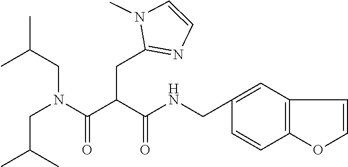 [SMILES: CC(C)CN(CC(C)C)C(=O)C(Cc1nccn1C)C(=O)NCc1ccc2occc2c1] | Cmpd. A 7 |
| 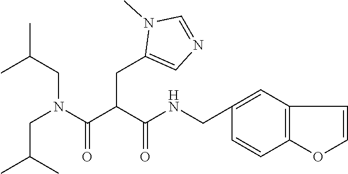 [SMILES: CC(C)CN(CC(C)C)C(=O)C(Cc1cncn1C)C(=O)NCc1ccc2occc2c1] | Cmpd. A 8 |
| 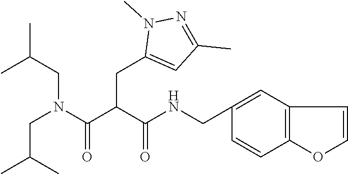 [SMILES: Cc1cc(CC(C(=O)NCc2ccc3occc3c2)C(=O)N(CC(C)C)CC(C)C)n(C)n1] | Cmpd. A 9 |
| 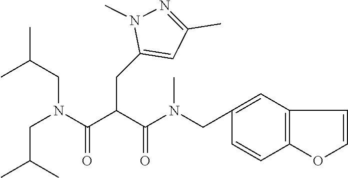 [SMILES: Cc1cc(CC(C(=O)N(C)Cc2ccc3occc3c2)C(=O)N(CC(C)C)CC(C)C)n(C)n1] | Cmpd. A 10 |
| 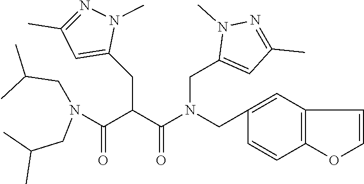 [SMILES: Cc1cc(CC(C(=O)N(Cc2ccc3occc3c2)Cc2cc(C)nn2C)C(=O)N(CC(C)C)CC(C)C)n(C)n1] | Cmpd. A 11 |
| 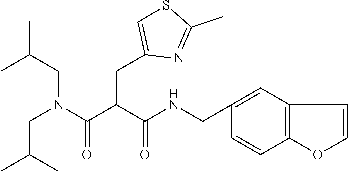 [SMILES: Cc1nc(CC(C(=O)NCc2ccc3occc3c2)C(=O)N(CC(C)C)CC(C)C)cs1] | Cmpd. A 12 |
| 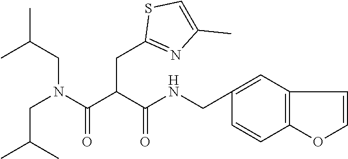 [SMILES: Cc1csc(CC(C(=O)NCc2ccc3occc3c2)C(=O)N(CC(C)C)CC(C)C)n1] | Cmpd. A 13 |
| 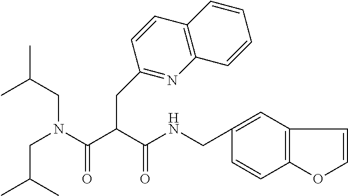 [SMILES: CC(C)CN(CC(C)C)C(=O)C(Cc1ccc2ccccc2n1)C(=O)NCc1ccc2occc2c1] | Cmpd. A 14 |
| 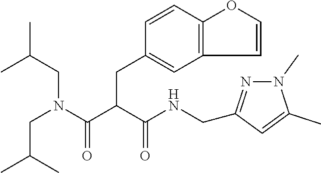 [SMILES: Cc1cc(CNC(=O)C(Cc2ccc3occc3c2)C(=O)N(CC(C)C)CC(C)C)nn1C] | Cmpd. A 15 |
| 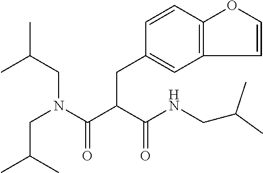 [SMILES: CC(C)CNC(=O)C(Cc1ccc2occc2c1)C(=O)N(CC(C)C)CC(C)C] | Cmpd. A 16 |
| 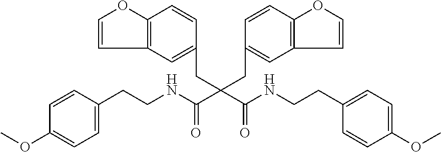 | Cmpd. A 17 |
| 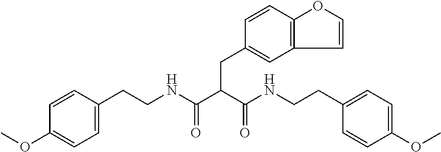 | Cmpd. A 18 |
| 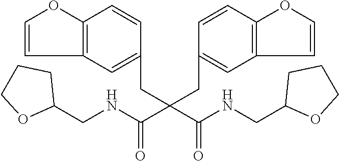 [SMILES: O=C(NCC1CCCO1)C(Cc1ccc2occc2c1)(Cc1ccc2occc2c1)C(=O)NCC1CCCO1] | Cmpd. A 19 |
| 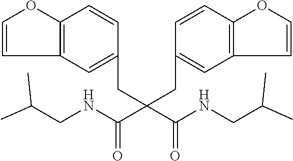 [SMILES: CC(C)CNC(=O)C(Cc1ccc2occc2c1)(Cc1ccc2occc2c1)C(=O)NCC(C)C] | Cmpd. A 20 |
| 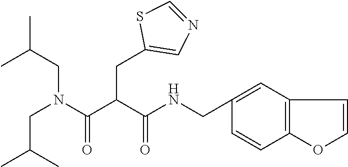 [SMILES: CC(C)CN(CC(C)C)C(=O)C(Cc1cncs1)C(=O)NCc1ccc2occc2c1] | Cmpd. A 21 |
| 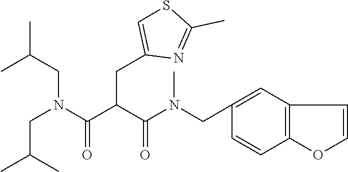 [SMILES: Cc1nc(CC(C(=O)N(C)Cc2ccc3occc3c2)C(=O)N(CC(C)C)CC(C)C)cs1] | Cmpd. A 22 |
| 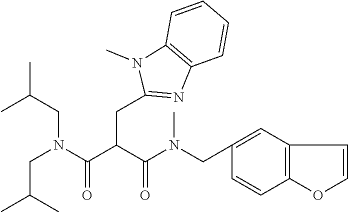 [SMILES: CC(C)CN(CC(C)C)C(=O)C(Cc1nc2ccccc2n1C)C(=O)N(C)Cc1ccc2occc2c1] | Cmpd. A 23 |
| 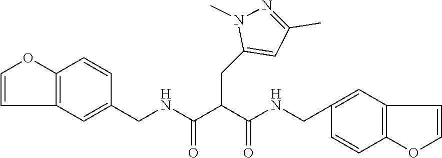 [SMILES: Cc1cc(CC(C(=O)NCc2ccc3occc3c2)C(=O)NCc2ccc3occc3c2)n(C)n1] | Cmpd. A 24 |
| 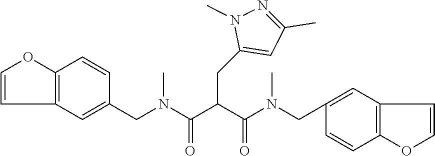 [SMILES: Cc1cc(CC(C(=O)N(C)Cc2ccc3occc3c2)C(=O)N(C)Cc2ccc3occc3c2)n(C)n1] | Cmpd. A 25 |
| 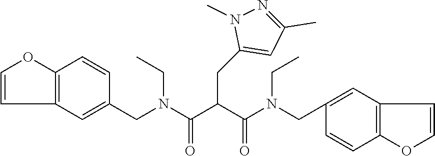 [SMILES: CCN(Cc1ccc2occc2c1)C(=O)C(Cc1cc(C)nn1C)C(=O)N(CC)Cc1ccc2occc2c1] | Cmpd. A 26 |
| 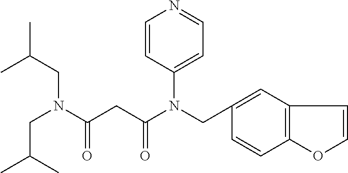 [SMILES: CC(C)CN(CC(C)C)C(=O)CC(=O)N(Cc1ccc2occc2c1)c1ccncc1] | Cmpd. A 27 |
| 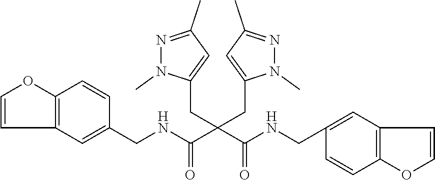 [SMILES: Cc1cc(CC(Cc2cc(C)nn2C)(C(=O)NCc2ccc3occc3c2)C(=O)NCc2ccc3occc3c2)n(C)n1] | Cmpd. A 28 |
| 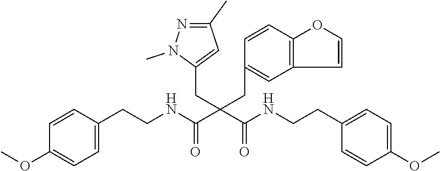 | Cmpd. A 29 |
| 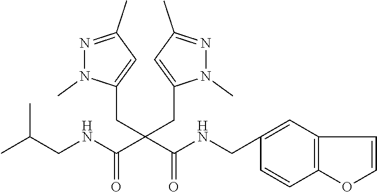 [SMILES: Cc1cc(CC(Cc2cc(C)nn2C)(C(=O)NCc2ccc3occc3c2)C(=O)NCC(C)C)n(C)n1] | Cmpd. A 30 |
| 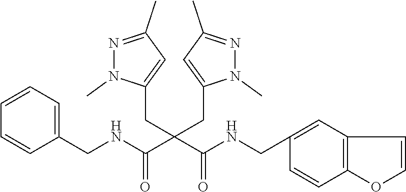 [SMILES: Cc1cc(CC(Cc2cc(C)nn2C)(C(=O)NCc2ccccc2)C(=O)NCc2ccc3occc3c2)n(C)n1] | Cmpd. A 31 |
| 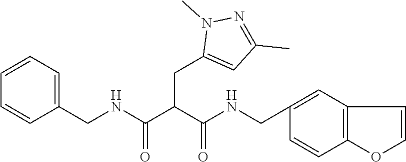 [SMILES: Cc1cc(CC(C(=O)NCc2ccccc2)C(=O)NCc2ccc3occc3c2)n(C)n1] | Cmpd. A 32 |
| 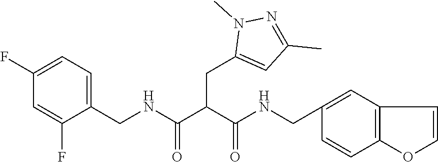 [SMILES: Cc1cc(CC(C(=O)NCc2ccc3occc3c2)C(=O)NCc2ccc(F)cc2F)n(C)n1] | Cmpd. A 33 |
| 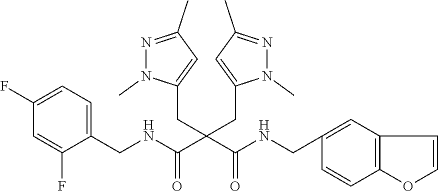 [SMILES: Cc1cc(CC(Cc2cc(C)nn2C)(C(=O)NCc2ccc3occc3c2)C(=O)NCc2ccc(F)cc2F)n(C)n1] | Cmpd. A 34 |
| 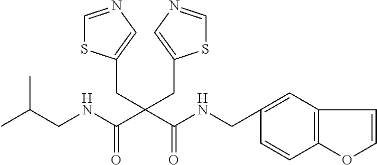 [SMILES: CC(C)CNC(=O)C(Cc1cncs1)(Cc1cncs1)C(=O)NCc1ccc2occc2c1] | Cmpd. A 35 |
| 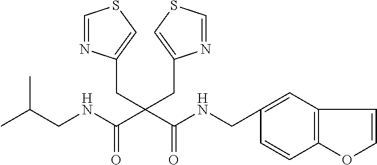 [SMILES: CC(C)CNC(=O)C(Cc1cscn1)(Cc1cscn1)C(=O)NCc1ccc2occc2c1] | Cmpd. A 36 |
| 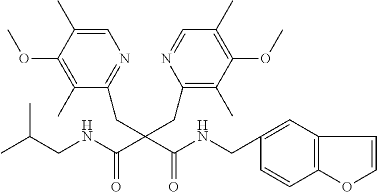 [SMILES: COc1c(C)cnc(CC(Cc2ncc(C)c(OC)c2C)(C(=O)NCc2ccc3occc3c2)C(=O)NCC(C)C)c1C] | Cmpd. A 37 |
| 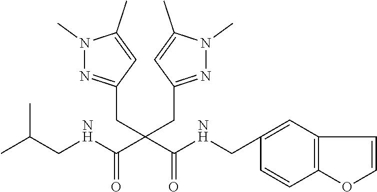 [SMILES: Cc1cc(CC(Cc2cc(C)n(C)n2)(C(=O)NCc2ccc3occc3c2)C(=O)NCC(C)C)nn1C] | Cmpd. A 38 |

In other embodiments the ICI may be represented by the formula VI:

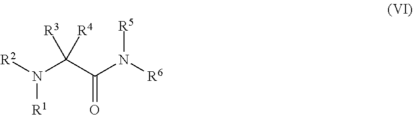

where:
each R¹, R², R³, R⁴, R⁵, and R⁶ independently is selected from the group consisting of H, optionally substituted C₁-C₈ alkyl, optionally substituted C₃-C₈ cycloalkyl, optionally substituted cycloalkylalkyl, optionally substituted aryl, optionally substituted aralkyl, optionally substituted heteroaryl, optionally substituted heteroaralkyl, optionally substituted heterocyclo, optionally substituted heterocycloalkylalkyl, —SOₙ(R), and —(CH₂)₀₋₆COₙ(CH₂)₀₋₃—R;
wherein at least one of R¹, R², R³, R⁴, R⁵, and R⁶ is —S(O)ₙ—(CH₂)₀₋₃-(benzofuranyl), —(CH₂)₁₋₅—N(R)(—SO₂-benzofuranyl), —C(O)ₙ—(CH₂)₀₋₃-(benzofuranyl), —(CH₂)₁₋₅—N(R)(C(═O)-(benzofuranyl), or C₁-C₆ alkylene-(benzofuranyl), wherein said benzofuranyl substituent may be substituted with an optional substituent, and wherein the benzofuranyl group is not fused as part of a tricyclic system;
each optional substituent is selected from the group consisting of halo, —CN, —NO₂, —COₙR, —OC(═O)R, —C(═O)N(R)₂, —C(═S)R, —C(═S)N(R)₂, —SOₙN(R)₂, —SR, —SOₙR, —N(R)₂, —N(R)COₙR, —NRS(═O)ₙR, —NRC[═N(R)]N(R)₂, —N(R)N(R)COₙR, —NRPOₙN(R)₂, —NRPOₙOR, oxo, ═N—OR, ═N—N(R)₂, ═NR, ═NNRC(O)N(R)₂, ═NNRCOₙR, ═NNRS(O)ₙN(R)₂, ═NNRS(O)ₙ(R)C₁-C₈ alkyl, —OR, C₁-C₈ alkyl, C₂-C₆ alkenyl, C₂-C₆ alkynyl, C₃-C₈ cycloalkyl, C₅-C₈ cycloalkenyl, heterocyclo, aryl, and heteroaryl;
each R is independently selected from the group consisting of: H, C₁-C₈ alkyl, aryl, aralkyl, heteroaryl, and heteroaralkyl, and when R is directly bound to a nitrogen, R may additionally be selected from —C(O)ₙalkyl, —S(O)ₙalkyl, —C(O)ₙ-aryl, —S(═O)ₙ-aryl, or —S(═O)ₙ-heteroaryl, wherein when R is a group selected from aryl, aralkyl, heteroaryl, heteroaralkyl, —C(O)ₙ-aryl, —S(═O)ₙ-aryl, or —S(═O)ₙ-heteroaryl, the group may substituted with one or more independently selected C₁-C₆ alkyls;
each n is independently 1 or 2;
provided that at least two of R¹, R², R3, R⁴, R⁵, and R⁶ are not H;
provided that neither R⁵ nor R⁶ forms a ring with R¹, R², R³, or R⁴. The compound may be

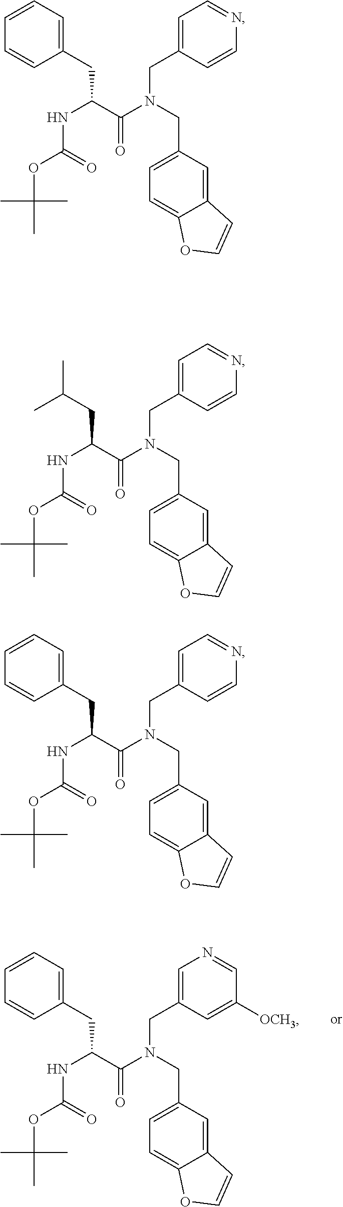

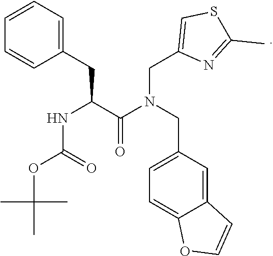 [SMILES: Cc1nc(CN(Cc2ccc3occc3c2)C(=O)[C@H](Cc2ccccc2)NC(=O)OC(C)(C)C)cs1]

In a specific embodiment, the ICI is a compound where:
each R¹, R², R³, R⁴, R⁵, and R⁶ independently is selected from the group consisting of H, optionally substituted C₁-C₈ alkyl, optionally substituted C₃-C₈ cycloalkyl, optionally substituted cycloalkylalkyl, optionally substituted aryl, optionally substituted aralkyl, optionally substituted heteroaryl, optionally substituted heteroaralkyl, optionally substituted heterocyclo, optionally substituted heterocycloalkylalkyl, —SOₙ(R), and —(CH₂)₀₋₆COₙ(CH₂)₀₋₃—R;
wherein at least one of R¹, R², R³, R⁴, R⁵, and R⁶ is —SOₙ-(benzofuranyl), —C(═O)-(benzofuranyl), —C(═O)O(CH₂)_{1_3}-(benzofuranyl), or C₁-C₆ alkylene-(benzofuranyl), wherein said benzofuranyl substituent may be substituted with an optional substituent and wherein the benzofuranyl group is not fused as part of a tricyclic system;
each optional substituent is selected from the group consisting of halo, —OC(═O)ₙR, —C(═O)N(R)₂, —C(═S)R, —C(═S)N(R)₂, —SOₙN(R)₂, —SR, —SOₙR, —N(R)₂, —N(R)COₙR, —NRS(═O)ₙR, —NRC[═N(R)]N(R)₂, —N(R)N(R)COₙR, —NRPOₙN(R)₂, —NRPOₙOR, oxo, —OR, C₁-C₈ alkyl, C₂-C₆ alkenyl, C₃-C₈ cycloalkyl, C₅-C₈ cycloalkenyl, heterocyclo, aryl, and heteroaryl;
each R is independently selected from the group consisting of: H, C₁-C₈ alkyl, aryl, aralkyl, heteroaryl, and heteroaralkyl, and when R is directly bound to a nitrogen, R may additionally be selected from —C(O)ₙalkyl, —S(O)ₙalkyl, —C(O)ₙ-aryl, —S(═O)ₙ-aryl, or —S(═O)ₙ-heteroaryl, wherein when R is a group selected from aryl, aralkyl, heteroaryl, heteroaralkyl, —C(O)ₙ-aryl, —S(═O)ₙ-aryl, or —S(═O)ₙ-heteroaryl, the group may substituted with one or more independently selected C₁-C₆ alkyls;
each n is independently 1 or 2.

In those embodiments where a benzofuranyl substituent group (radical) is recited, any benzofuranyl group may be present. Alternatively, where a benzofuranyl group is recited the radical may be selected from any one or more of a 4-benzofuranyl, 5-benzofuranyl, 6-benzofuranyl, or a 7-benzofuranyl radical.

In some embodiments, R is independently selected from the group consisting of: H, C₁-C₈ alkyl, aryl, aralkyl, heteroaryl, and heteroaralkyl, and when R is directly bound to a nitrogen, R may additionally be selected from —C(O)ₙalkyl, —S(O)ₙalkyl, —C(O)ₙ-aryl, —S(═O)ₙ-aryl, or —S(═O)ₙ-heteroaryl, wherein when R is a group selected from aryl, aralkyl, heteroaryl, heteroaralkyl, —C(O)ₙ-aryl, —S(═O)ₙ-aryl, or —S(═O)ₙ-heteroaryl, the group may substituted with one or more independently selected C₁-C₆ alkyls. In other embodiments, each R is independently selected from the group consisting of: H, C₁-C₈ alkyl, aryl, aralkyl, heteroaryl, and heteroaralky, and when R is directly bound to a nitrogen, R may additionally be selected from —S(═O)ₙ-aryl, or —S(═O)ₙ-heteroaryl, wherein when R is a group selected from aryl, aralkyl, heteroaryl, heteroaralkyl, —S(═O)ₙ-aryl, or —S(═O)ₙ-heteroaryl, the group may substituted with one or more independently selected C₁-C₆ alkyls. In other embodiments, each R is independently selected from the group consisting of: H, C₁-C₈ alkyl, aryl, aralkyl, heteroaryl, and heteroaralky, wherein when R is a group selected from aryl, aralkyl, heteroaryl, heteroaralkyl, the group may substituted with one or more independently selected C₁-C₆ alkyls. In another embodiment, R is selected from the group consisting of C₁-C₈ alkyl, aryl, aralkyl, heteroaryl, and heteroaralky, each of which may be substituted with one or more independently selected C₁-C₆ alkyls.

In various embodiments, R⁵ is an otherwise unsubstituted CH₂-4-benzofuranyl, CH₂-5-benzofuranyl, CH₂-6-benzofuranyl, or CH₂-7-benzofuranyl. R³ can be H and R⁴ can be selected from the group consisting of H, optionally substituted C₁-C₈ alkyl, optionally substituted C₃-C₈ cycloalkyl, optionally substituted cycloalkylalkyl, optionally substituted aryl, optionally substituted aralkyl, optionally substituted heteroaryl, optionally substituted heteroaralkyl, optionally substituted heterocyclo, and optionally substituted heterocycloalkylalkyl. R¹ can be optionally substituted C₁-C₈ alkyl. R² can be optionally substituted C₁-C₈ alkyl. R⁴ can be optionally substituted alkyl or optionally substituted heteroaralkyl. R⁶ can be H, optionally substituted C₁-C₈ alkyl, or optionally substituted heteroaralkyl. R¹ and R² can be optionally substituted C₁-C₈ alkyl. R³ can be H, R⁴ can be H, optionally substituted C₁-C₈ alkyl or optionally substituted heteroaralkyl, and R⁶ can be H, optionally substituted C₁-C₈ alkyl or optionally substituted heteroaralkyl.

In some embodiments, R³ is an otherwise unsubstituted —CH₂-4-benzofuranyl, —CH₂-5-benzofuranyl, —CH₂-6-benzofuranyl, or —CH₂-7-benzofuranyl.

In some embodiments, R⁴ can be H or C₁-C₈ alkyl. In other embodiments, R⁴ can be H. In another embodiment, R⁴ can be H and R⁵ can be H.

In some embodiments, R⁶ can be selected from the group consisting of optionally substituted C₁-C₈ alkyl, optionally substituted C₃-C₈ cycloalkyl, optionally substituted cycloalkylalkyl, optionally substituted aryl, optionally substituted aralkyl, optionally substituted heteroaryl, optionally substituted heteroaralkyl, optionally substituted heterocyclo, and optionally substituted heterocycloalkylalkyl.

In some embodiments, R¹ can be an otherwise unsubstituted —CH₂-4-benzofuranyl, —CH₂-5-benzofuranyl, —CH₂-6-benzofuranyl, or —CH₂-7-benzofuranyl. In another embodiment, only one of R1 and R3 is —CH₂-4-benzofuranyl, —CH₂-5-benzofuranyl, —CH₂-6-benzofuranyl, or —CH₂-7-benzofuranyl.

In certain embodiments, a compound is selected from the compounds listed in the table below.

In various embodiments, one or both of R³ and R⁴ is heteroarylalkyl.

In some embodiments, one or both of R³ and R⁴ is heteroarylmethyl.

By way of illustration, exemplary compounds of formula (VI) are shown in the table below, although it will be recognized that these examples are merely illustrative and not limiting. All shown compounds have an IC₅₀ less than 100 nM for the metabolism of dibenzylfluorescein (DFB) by human liver microsomes (Xeno Tech, LLC, Lenexa, Kans.). Where tested, the compound also have an IC₅₀ less than 100 nM for the inhibition of DFB utilization by CYP 3A4.

TABLE 2

| | |
|---|---|
| 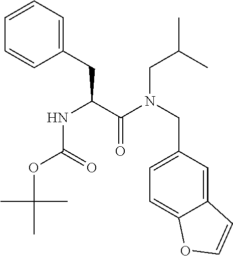 [SMILES: CC(C)CN(Cc1ccc2occc2c1)C(=O)[C@H](Cc1ccccc1)NC(=O)OC(C)(C)C] | Cmpd. B 1 |
| 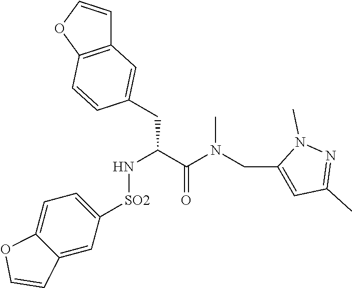 [SMILES: Cc1cc(CN(C)C(=O)[C@@H](Cc2ccc3occc3c2)NS(=O)(=O)c2ccc3occc3c2)n(C)n1] | Cmpd. B 2 |
| 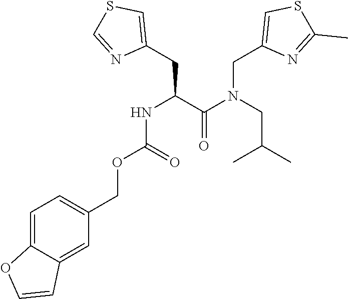 [SMILES: Cc1nc(CN(CC(C)C)C(=O)[C@H](Cc2cscn2)NC(=O)OCc2ccc3occc3c2)cs1] | Cmpd. B 3 |
| 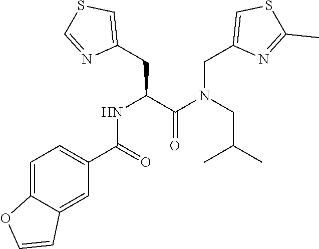 [SMILES: Cc1nc(CN(CC(C)C)C(=O)[C@H](Cc2cscn2)NC(=O)c2ccc3occc3c2)cs1] | Cmpd. B 4 |
| 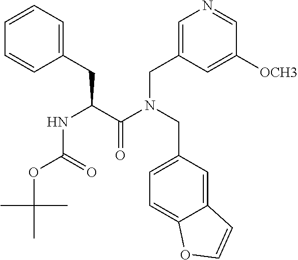 [SMILES: COc1cncc(CN(Cc2ccc3occc3c2)C(=O)[C@H](Cc2ccccc2)NC(=O)OC(C)(C)C)c1] | Cmpd. B 5 |
| 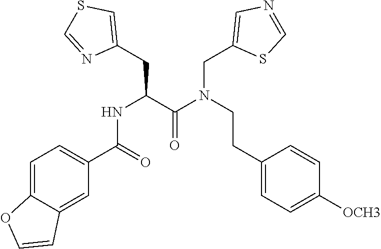 [SMILES: COc1ccc(CCN(Cc2cncs2)C(=O)[C@H](Cc2cscn2)NC(=O)c2ccc3occc3c2)cc1] | Cmpd. B 6 |
| 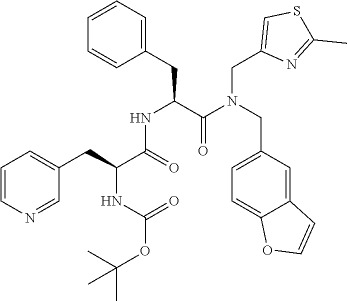 [SMILES: Cc1nc(CN(Cc2ccc3occc3c2)C(=O)[C@H](Cc2ccccc2)NC(=O)[C@H](Cc2cccnc2)NC(=O)OC(C)(C)C)cs1] | Cmpd. B 7 |
| 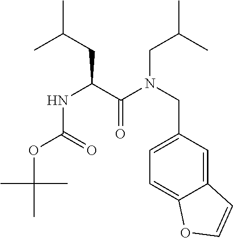 [SMILES: CC(C)C[C@H](NC(=O)OC(C)(C)C)C(=O)N(Cc1ccc2occc2c1)CC(C)C] | Cmpd. B 8 |
| 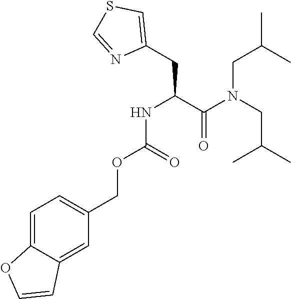 [SMILES: CC(C)CN(CC(C)C)C(=O)[C@H](Cc1cscn1)NC(=O)OCc1ccc2occc2c1] | Cmpd. B 9 |
| 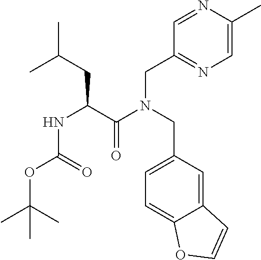 [SMILES: Cc1cnc(CN(Cc2ccc3occc3c2)C(=O)[C@H](CC(C)C)NC(=O)OC(C)(C)C)cn1] | Cmpd. B 10 |
| 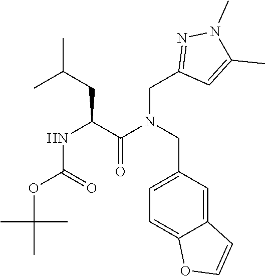 [SMILES: Cc1cc(CN(Cc2ccc3occc3c2)C(=O)[C@H](CC(C)C)NC(=O)OC(C)(C)C)nn1C] | Cmpd. B 11 |
| 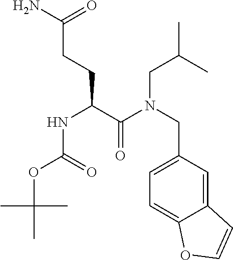 [SMILES: CC(C)CN(Cc1ccc2occc2c1)C(=O)[C@H](CCC(N)=O)NC(=O)OC(C)(C)C] | Cmpd. B 12 |
| 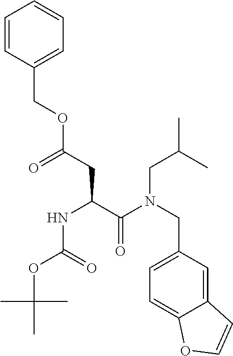 [SMILES: CC(C)CN(Cc1ccc2occc2c1)C(=O)[C@H](CC(=O)OCc1ccccc1)NC(=O)OC(C)(C)C] | Cmpd. B 13 |
| 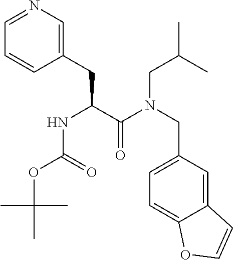 [SMILES: CC(C)CN(Cc1ccc2occc2c1)C(=O)[C@H](Cc1cccnc1)NC(=O)OC(C)(C)C] | Cmpd. B 14 |
| 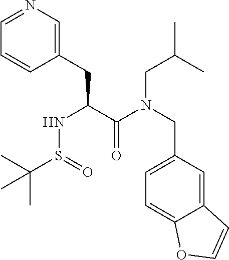 [SMILES: CC(C)CN(Cc1ccc2occc2c1)C(=O)[C@H](Cc1cccnc1)NS(=O)C(C)(C)C] | Cmpd. B 15 |
| 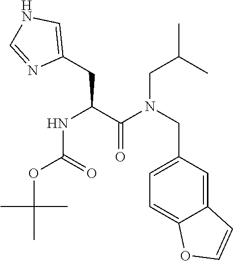 [SMILES: CC(C)CN(Cc1ccc2occc2c1)C(=O)[C@H](Cc1c[nH]cn1)NC(=O)OC(C)(C)C] | Cmpd. B 16 |
| 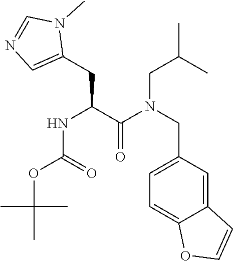 [SMILES: CC(C)CN(Cc1ccc2occc2c1)C(=O)[C@H](Cc1cncn1C)NC(=O)OC(C)(C)C] | Cmpd. B 17 |
| 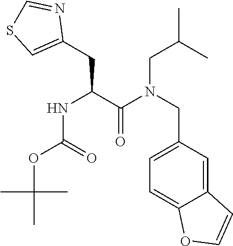 [SMILES: CC(C)CN(Cc1ccc2occc2c1)C(=O)[C@H](Cc1cscn1)NC(=O)OC(C)(C)C] | Cmpd. B 18 |
| 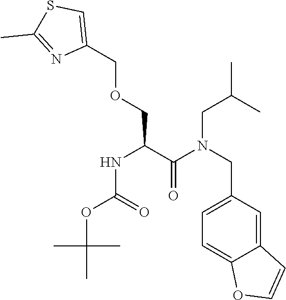 [SMILES: Cc1nc(COC[C@H](NC(=O)OC(C)(C)C)C(=O)N(Cc2ccc3occc3c2)CC(C)C)cs1] | Cmpd. B 19 |
| 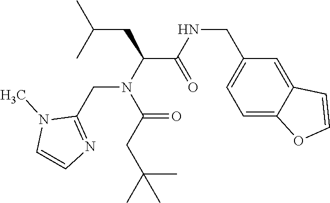 [SMILES: CC(C)C[C@@H](C(=O)NCc1ccc2occc2c1)N(Cc1nccn1C)C(=O)CC(C)(C)C] | Cmpd. B 20 |
| 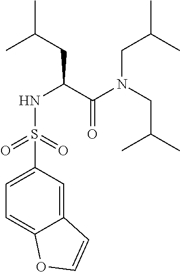 [SMILES: CC(C)C[C@H](NS(=O)(=O)c1ccc2occc2c1)C(=O)N(CC(C)C)CC(C)C] | Cmpd. B 21 |
| 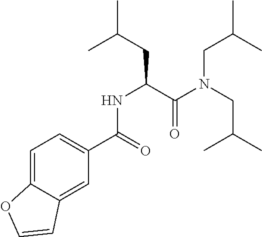 [SMILES: CC(C)C[C@H](NC(=O)c1ccc2occc2c1)C(=O)N(CC(C)C)CC(C)C] | Cmpd. B 22 |
| 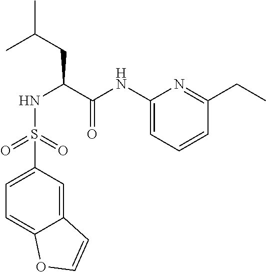 [SMILES: CCc1cccc(NC(=O)[C@H](CC(C)C)NS(=O)(=O)c2ccc3occc3c2)n1] | Cmpd. B 23 |
| 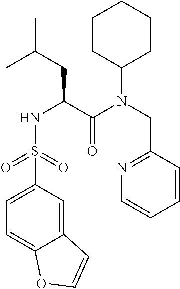 [SMILES: CC(C)C[C@H](NS(=O)(=O)c1ccc2occc2c1)C(=O)N(Cc1ccccn1)C1CCCCC1] | Cmpd. B 24 |
| 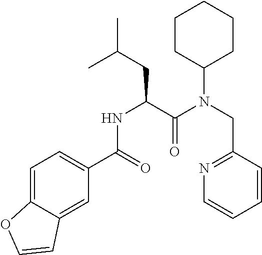 [SMILES: CC(C)C[C@H](NC(=O)c1ccc2occc2c1)C(=O)N(Cc1ccccn1)C1CCCCC1] | Cmpd. B 25 |
| 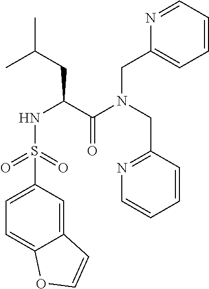 [SMILES: CC(C)C[C@H](NS(=O)(=O)c1ccc2occc2c1)C(=O)N(Cc1ccccn1)Cc1ccccn1] | Cmpd. B 26 |
| 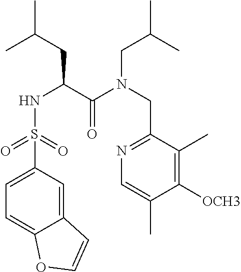 [SMILES: COc1c(C)cnc(CN(CC(C)C)C(=O)[C@H](CC(C)C)NS(=O)(=O)c2ccc3occc3c2)c1C] | Cmpd. B 27 |
| 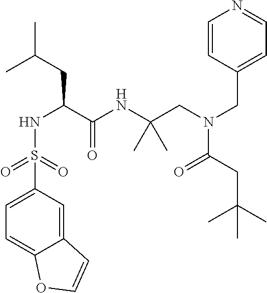 [SMILES: CC(C)C[C@H](NS(=O)(=O)c1ccc2occc2c1)C(=O)NC(C)(C)CN(Cc1ccncc1)C(=O)CC(C)(C)C] | Cmpd. B 28 |
| 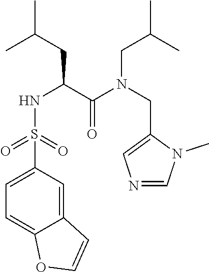 [SMILES: CC(C)C[C@H](NS(=O)(=O)c1ccc2occc2c1)C(=O)N(Cc1cncn1C)CC(C)C] | Cmpd. B 29 |
| 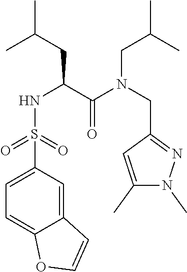 [SMILES: Cc1cc(CN(CC(C)C)C(=O)[C@H](CC(C)C)NS(=O)(=O)c2ccc3occc3c2)nn1C] | Cmpd. B 30 |
| 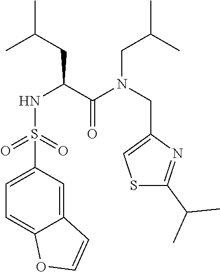 [SMILES: CC(C)C[C@H](NS(=O)(=O)c1ccc2occc2c1)C(=O)N(Cc1csc(C(C)C)n1)CC(C)C] | Cmpd. B 31 |
| 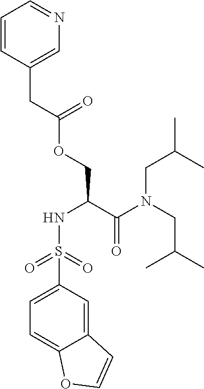 [SMILES: CC(C)CN(CC(C)C)C(=O)[C@H](COC(=O)Cc1cccnc1)NS(=O)(=O)c1ccc2occc2c1] | Cmpd. B 32 |
| 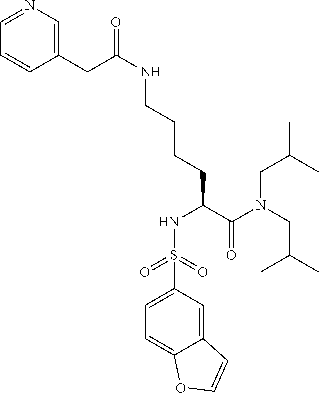 [SMILES: CC(C)CN(CC(C)C)C(=O)[C@H](CCCCNC(=O)Cc1cccnc1)NS(=O)(=O)c1ccc2occc2c1] | Cmpd. B 33 |
| 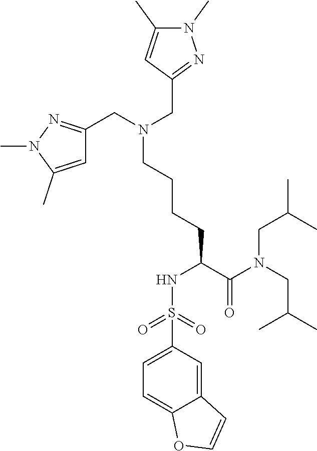 [SMILES: Cc1cc(CN(CCCC[C@H](NS(=O)(=O)c2ccc3occc3c2)C(=O)N(CC(C)C)CC(C)C)Cc2cc(C)n(C)n2)nn1C] | Cmpd. B 34 |
| 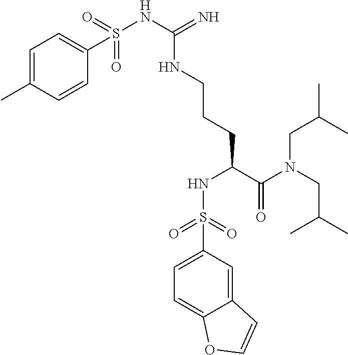 [SMILES: Cc1ccc(S(=O)(=O)NC(=N)NCCC[C@H](NS(=O)(=O)c2ccc3occc3c2)C(=O)N(CC(C)C)CC(C)C)cc1] | Cmpd. B 35 |
| 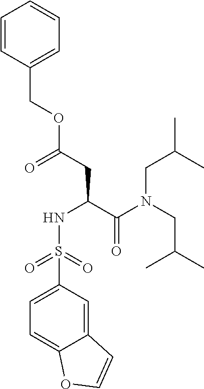 [SMILES: CC(C)CN(CC(C)C)C(=O)[C@H](CC(=O)OCc1ccccc1)NS(=O)(=O)c1ccc2occc2c1] | Cmpd. B 36 |
| 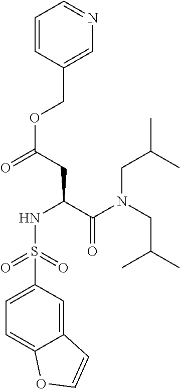 [SMILES: CC(C)CN(CC(C)C)C(=O)[C@H](CC(=O)OCc1cccnc1)NS(=O)(=O)c1ccc2occc2c1] | Cmpd. B 37 |
| 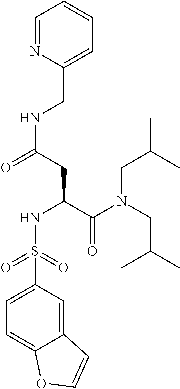 [SMILES: CC(C)CN(CC(C)C)C(=O)[C@H](CC(=O)NCc1ccccn1)NS(=O)(=O)c1ccc2occc2c1] | Cmpd. B 38 |
| 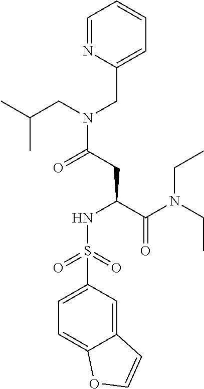 [SMILES: CCN(CC)C(=O)[C@H](CC(=O)N(Cc1ccccn1)CC(C)C)NS(=O)(=O)c1ccc2occc2c1] | Cmpd. B 39 |
| 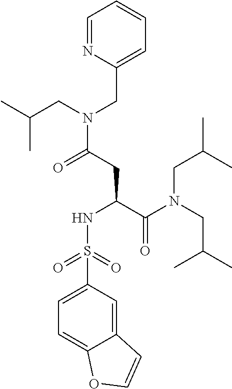 [SMILES: CC(C)CN(Cc1ccccn1)C(=O)C[C@H](NS(=O)(=O)c1ccc2occc2c1)C(=O)N(CC(C)C)CC(C)C] | Cmpd. B 40 |
| 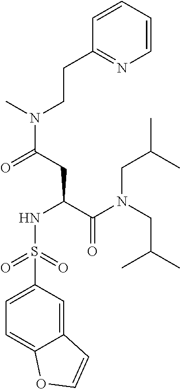 [SMILES: CC(C)CN(CC(C)C)C(=O)[C@H](CC(=O)N(C)CCc1ccccn1)NS(=O)(=O)c1ccc2occc2c1] | Cmpd. B 41 |
| 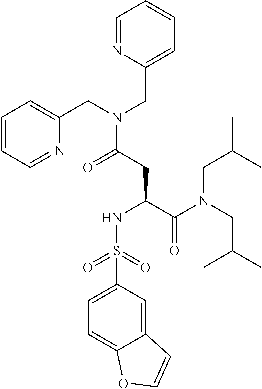 [SMILES: CC(C)CN(CC(C)C)C(=O)[C@H](CC(=O)N(Cc1ccccn1)Cc1ccccn1)NS(=O)(=O)c1ccc2occc2c1] | Cmpd. B 42 |
| 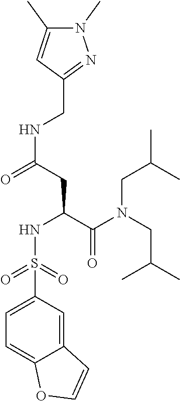 [SMILES: Cc1cc(CNC(=O)C[C@H](NS(=O)(=O)c2ccc3occc3c2)C(=O)N(CC(C)C)CC(C)C)nn1C] | Cmpd. B 43 |
| 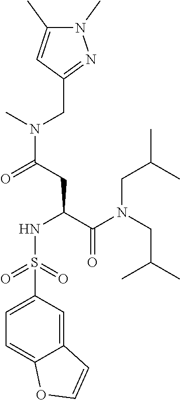 [SMILES: Cc1cc(CN(C)C(=O)C[C@H](NS(=O)(=O)c2ccc3occc3c2)C(=O)N(CC(C)C)CC(C)C)nn1C] | Cmpd. B 44 |
| 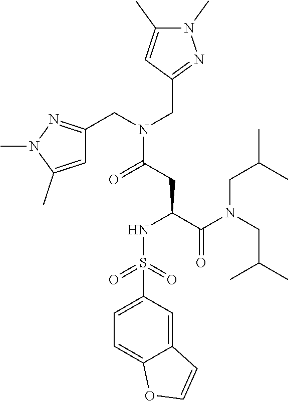 [SMILES: Cc1cc(CN(Cc2cc(C)n(C)n2)C(=O)C[C@H](NS(=O)(=O)c2ccc3occc3c2)C(=O)N(CC(C)C)CC(C)C)nn1C] | Cmpd. B 45 |
| 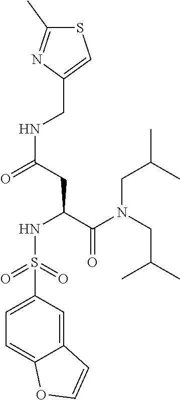 [SMILES: Cc1nc(CNC(=O)C[C@H](NS(=O)(=O)c2ccc3occc3c2)C(=O)N(CC(C)C)CC(C)C)cs1] | Cmpd. B 46 |
| 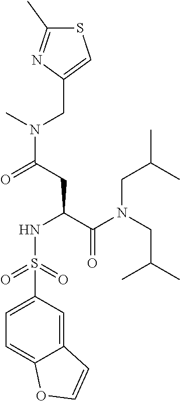 [SMILES: Cc1nc(CN(C)C(=O)C[C@H](NS(=O)(=O)c2ccc3occc3c2)C(=O)N(CC(C)C)CC(C)C)cs1] | Cmpd. B 47 |
| 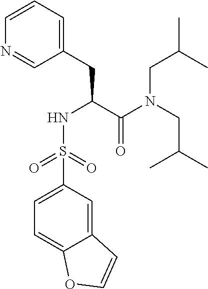 [SMILES: CC(C)CN(CC(C)C)C(=O)[C@H](Cc1cccnc1)NS(=O)(=O)c1ccc2occc2c1] | Cmpd. B 48 |
| 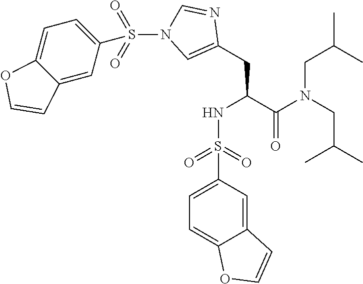 [SMILES: CC(C)CN(CC(C)C)C(=O)[C@H](Cc1cn(S(=O)(=O)c2ccc3occc3c2)cn1)NS(=O)(=O)c1ccc2occc2c1] | Cmpd. B 49 |
| 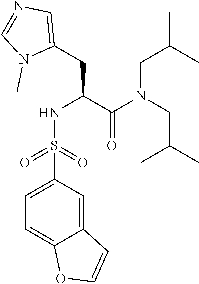 [SMILES: CC(C)CN(CC(C)C)C(=O)[C@H](Cc1cncn1C)NS(=O)(=O)c1ccc2occc2c1] | Cmpd. B 50 |
| 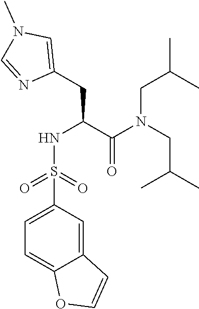 [SMILES: CC(C)CN(CC(C)C)C(=O)[C@H](Cc1cn(C)cn1)NS(=O)(=O)c1ccc2occc2c1] | Cmpd. B 51 |
| 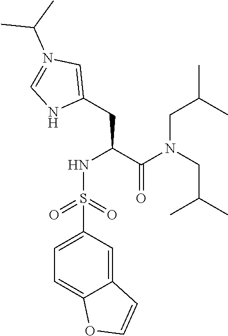 | Cmpd. B 52 |
| 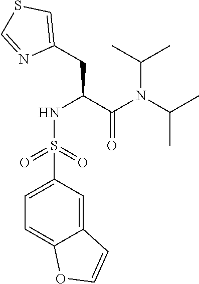 [SMILES: CC(C)N(C(=O)[C@H](Cc1cscn1)NS(=O)(=O)c1ccc2occc2c1)C(C)C] | Cmpd. B 53 |
| 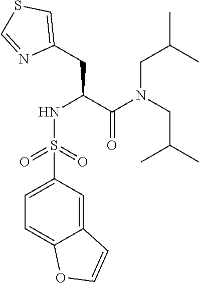 [SMILES: CC(C)CN(CC(C)C)C(=O)[C@H](Cc1cscn1)NS(=O)(=O)c1ccc2occc2c1] | Cmpd. B 54 |
| 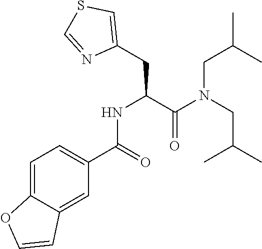 [SMILES: CC(C)CN(CC(C)C)C(=O)[C@H](Cc1cscn1)NC(=O)c1ccc2occc2c1] | Cmpd. B 55 |
| 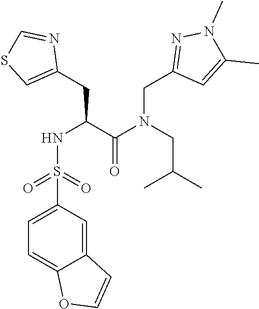 [SMILES: Cc1cc(CN(CC(C)C)C(=O)[C@H](Cc2cscn2)NS(=O)(=O)c2ccc3occc3c2)nn1C] | Cmpd. B 56 |
| 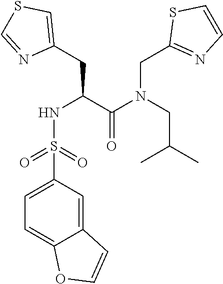 [SMILES: CC(C)CN(Cc1nccs1)C(=O)[C@H](Cc1cscn1)NS(=O)(=O)c1ccc2occc2c1] | Cmpd. B 57 |
| 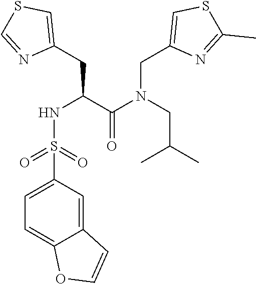 [SMILES: Cc1nc(CN(CC(C)C)C(=O)[C@H](Cc2cscn2)NS(=O)(=O)c2ccc3occc3c2)cs1] | Cmpd. B 58 |
| 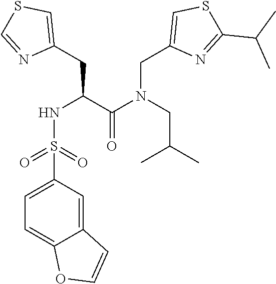 [SMILES: CC(C)CN(Cc1csc(C(C)C)n1)C(=O)[C@H](Cc1cscn1)NS(=O)(=O)c1ccc2occc2c1] | Cmpd. B 59 |
| 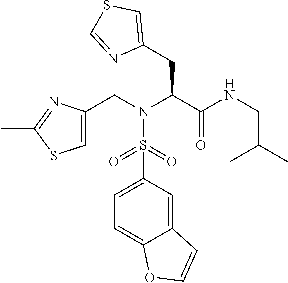 [SMILES: Cc1nc(CN([C@@H](Cc2cscn2)C(=O)NCC(C)C)S(=O)(=O)c2ccc3occc3c2)cs1] | Cmpd. B 60 |
| 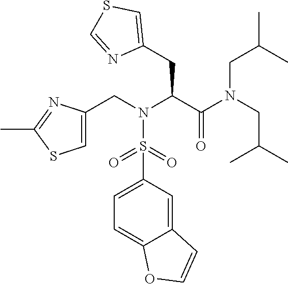 [SMILES: Cc1nc(CN([C@@H](Cc2cscn2)C(=O)N(CC(C)C)CC(C)C)S(=O)(=O)c2ccc3occc3c2)cs1] | Cmpd. B 61 |
| 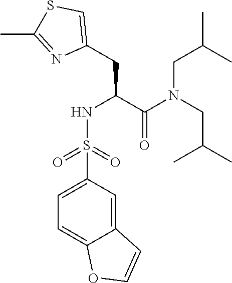 [SMILES: Cc1nc(C[C@H](NS(=O)(=O)c2ccc3occc3c2)C(=O)N(CC(C)C)CC(C)C)cs1] | Cmpd. B 62 |
| 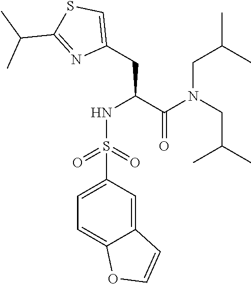 [SMILES: CC(C)CN(CC(C)C)C(=O)[C@H](Cc1csc(C(C)C)n1)NS(=O)(=O)c1ccc2occc2c1] | Cmpd. B 63 |
| 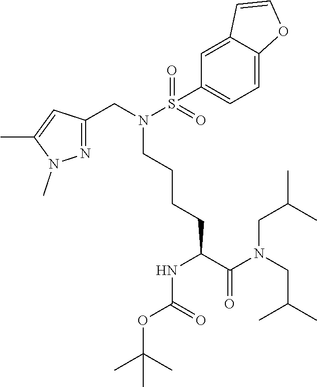 [SMILES: Cc1cc(CN(CCCC[C@H](NC(=O)OC(C)(C)C)C(=O)N(CC(C)C)CC(C)C)S(=O)(=O)c2ccc3occc3c2)nn1C] | Cmpd. B 64 |
| 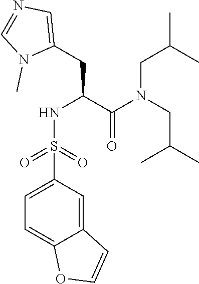 [SMILES: CC(C)CN(CC(C)C)C(=O)[C@H](Cc1cncn1C)NS(=O)(=O)c1ccc2occc2c1] | Cmpd. B 65 |
| 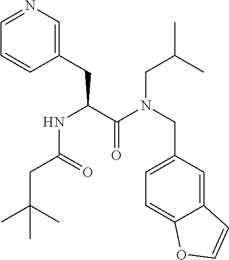 [SMILES: CC(C)CN(Cc1ccc2occc2c1)C(=O)[C@H](Cc1cccnc1)NC(=O)CC(C)(C)C] | Cmpd. B 66 |
| 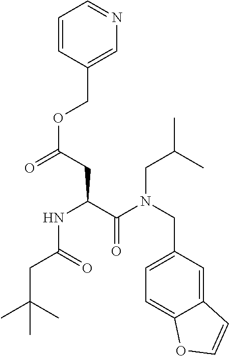 [SMILES: CC(C)CN(Cc1ccc2occc2c1)C(=O)[C@H](CC(=O)OCc1cccnc1)NC(=O)CC(C)(C)C] | Cmpd. B 67 |
| 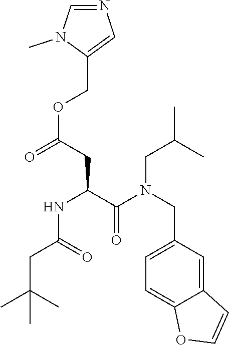 [SMILES: CC(C)CN(Cc1ccc2occc2c1)C(=O)[C@H](CC(=O)OCc1cncn1C)NC(=O)CC(C)(C)C] | Cmpd. B 68 |
| 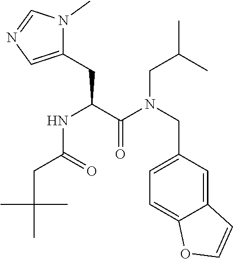 [SMILES: CC(C)CN(Cc1ccc2occc2c1)C(=O)[C@H](Cc1cncn1C)NC(=O)CC(C)(C)C] | Cmpd. B 69 |
| 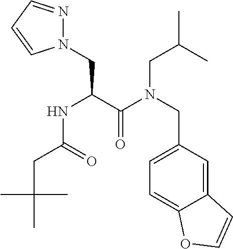 [SMILES: CC(C)CN(Cc1ccc2occc2c1)C(=O)[C@H](Cn1cccn1)NC(=O)CC(C)(C)C] | Cmpd. B 70 |
| 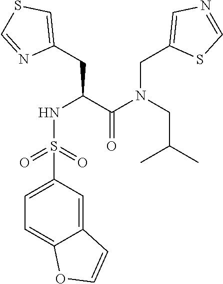 [SMILES: CC(C)CN(Cc1cncs1)C(=O)[C@H](Cc1cscn1)NS(=O)(=O)c1ccc2occc2c1] | Cmpd. B 71 |
| 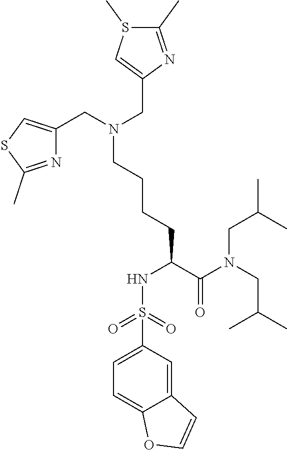 | Cmpd. B 72 |
| 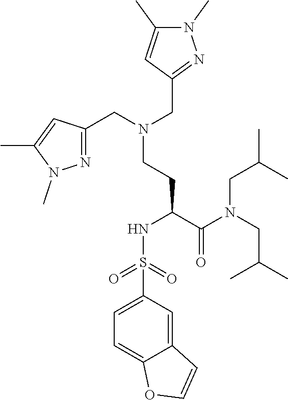 [SMILES: Cc1cc(CN(CC[C@H](NS(=O)(=O)c2ccc3occc3c2)C(=O)N(CC(C)C)CC(C)C)Cc2cc(C)n(C)n2)nn1C] | Cmpd. B 73 |
| 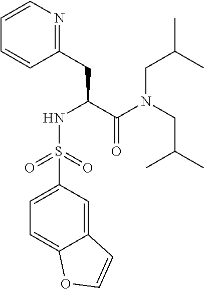 [SMILES: CC(C)CN(CC(C)C)C(=O)[C@H](Cc1ccccn1)NS(=O)(=O)c1ccc2occc2c1] | Cmpd. B 74 |
| 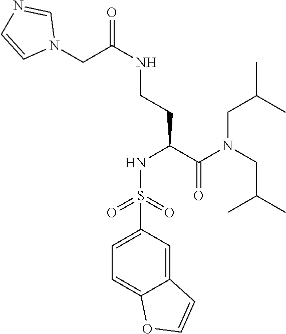 [SMILES: CC(C)CN(CC(C)C)C(=O)[C@H](CCNC(=O)Cn1ccnc1)NS(=O)(=O)c1ccc2occc2c1] | Cmpd. B 75 |
| 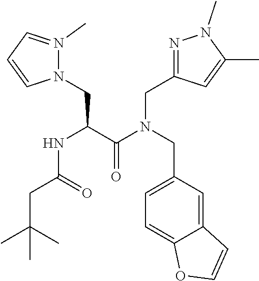 | Cmpd. B 76 |
| 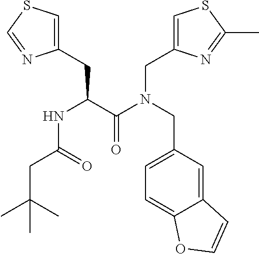 [SMILES: Cc1nc(CN(Cc2ccc3occc3c2)C(=O)[C@H](Cc2cscn2)NC(=O)CC(C)(C)C)cs1] | Cmpd. B 77 |
| 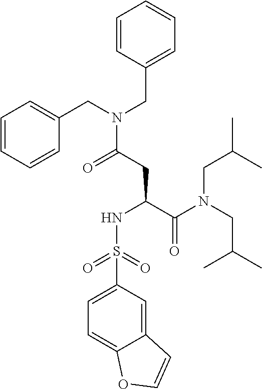 [SMILES: CC(C)CN(CC(C)C)C(=O)[C@H](CC(=O)N(Cc1ccccc1)Cc1ccccc1)NS(=O)(=O)c1ccc2occc2c1] | Cmpd. B 78 |
| 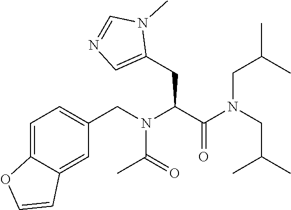 [SMILES: CC(=O)N(Cc1ccc2occc2c1)[C@@H](Cc1cncn1C)C(=O)N(CC(C)C)CC(C)C] | Cmpd. B 79 |
| 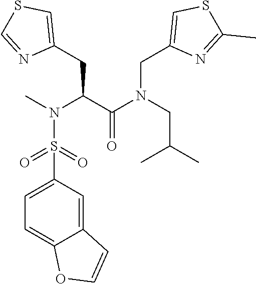 [SMILES: Cc1nc(CN(CC(C)C)C(=O)[C@H](Cc2cscn2)N(C)S(=O)(=O)c2ccc3occc3c2)cs1] | Cmpd. B 80 |
| 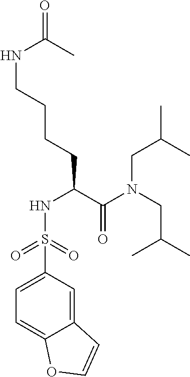 [SMILES: CC(=O)NCCCC[C@H](NS(=O)(=O)c1ccc2occc2c1)C(=O)N(CC(C)C)CC(C)C] | Cmpd. B 81 |
| 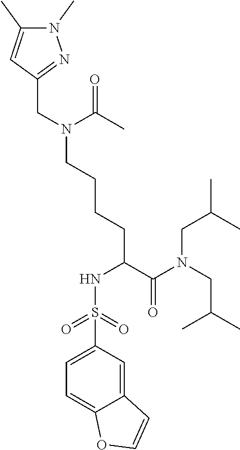 [SMILES: CC(=O)N(CCCCC(NS(=O)(=O)c1ccc2occc2c1)C(=O)N(CC(C)C)CC(C)C)Cc1cc(C)n(C)n1] | Cmpd. B 82 |
| 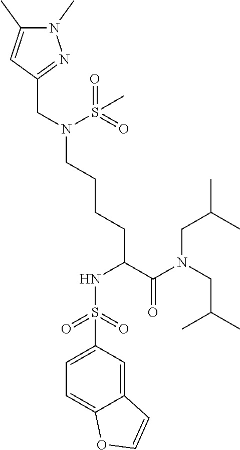 [SMILES: Cc1cc(CN(CCCCC(NS(=O)(=O)c2ccc3occc3c2)C(=O)N(CC(C)C)CC(C)C)S(C)(=O)=O)nn1C] | Cmpd. B 83 |
| 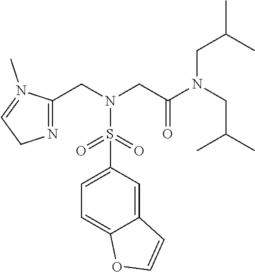 | Cmpd. B 84 |
| 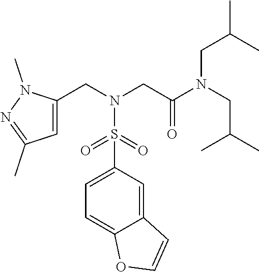 [SMILES: Cc1cc(CN(CC(=O)N(CC(C)C)CC(C)C)S(=O)(=O)c2ccc3occc3c2)n(C)n1] | Cmpd. B 85 |
| 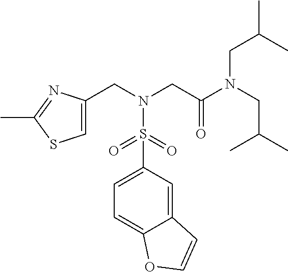 [SMILES: Cc1nc(CN(CC(=O)N(CC(C)C)CC(C)C)S(=O)(=O)c2ccc3occc3c2)cs1] | Cmpd. B 86 |
| 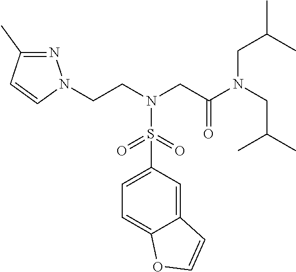 [SMILES: Cc1ccn(CCN(CC(=O)N(CC(C)C)CC(C)C)S(=O)(=O)c2ccc3occc3c2)n1] | Cmpd. B 87 |
| 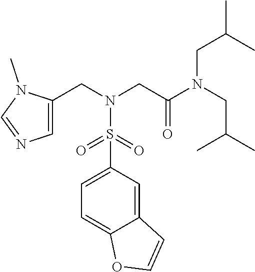 [SMILES: CC(C)CN(CC(C)C)C(=O)CN(Cc1cncn1C)S(=O)(=O)c1ccc2occc2c1] | Cmpd. B 88 |
| 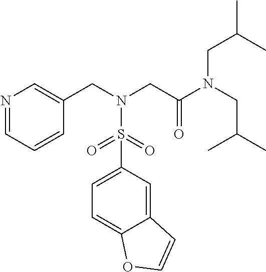 [SMILES: CC(C)CN(CC(C)C)C(=O)CN(Cc1cccnc1)S(=O)(=O)c1ccc2occc2c1] | Cmpd. B 89 |
| 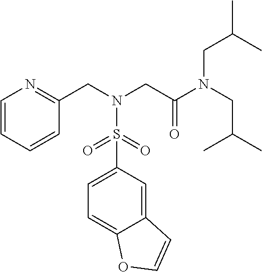 [SMILES: CC(C)CN(CC(C)C)C(=O)CN(Cc1ccccn1)S(=O)(=O)c1ccc2occc2c1] | Cmpd. B 90 |
| 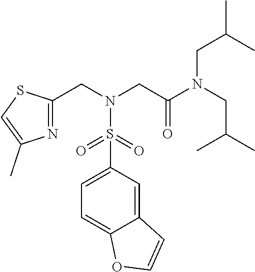 [SMILES: Cc1csc(CN(CC(=O)N(CC(C)C)CC(C)C)S(=O)(=O)c2ccc3occc3c2)n1] | Cmpd. B 91 |
| 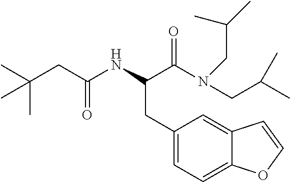 | Cmpd. B 92 |
| 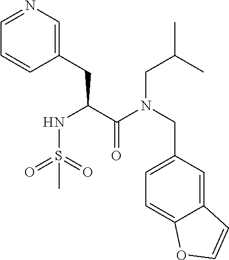 [SMILES: CC(C)CN(Cc1ccc2occc2c1)C(=O)[C@H](Cc1cccnc1)NS(C)(=O)=O] | Cmpd. B 93 |
| 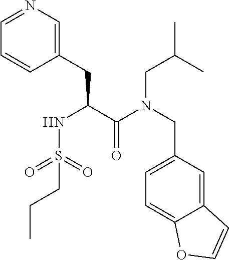 [SMILES: CCCS(=O)(=O)N[C@@H](Cc1cccnc1)C(=O)N(Cc1ccc2occc2c1)CC(C)C] | Cmpd. B 94 |
| 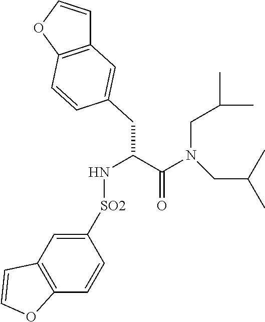 [SMILES: CC(C)CN(CC(C)C)C(=O)[C@@H](Cc1ccc2occc2c1)NS(=O)(=O)c1ccc2occc2c1] | Cmpd. B 95 |
| 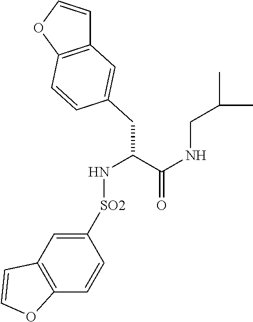 [SMILES: CC(C)CNC(=O)[C@@H](Cc1ccc2occc2c1)NS(=O)(=O)c1ccc2occc2c1] | Cmpd. B 96 |
| 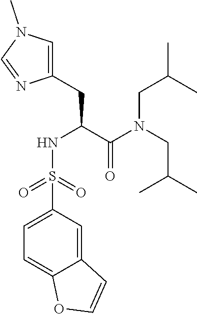 [SMILES: CC(C)CN(CC(C)C)C(=O)[C@H](Cc1cn(C)cn1)NS(=O)(=O)c1ccc2occc2c1] | Cmpd. B 97 |
| 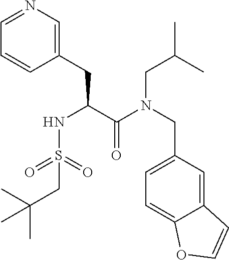 [SMILES: CC(C)CN(Cc1ccc2occc2c1)C(=O)[C@H](Cc1cccnc1)NS(=O)(=O)CC(C)(C)C] | Cmpd. B 98 |
| 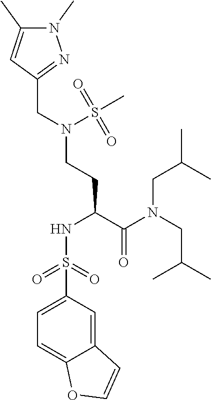 [SMILES: Cc1cc(CN(CC[C@H](NS(=O)(=O)c2ccc3occc3c2)C(=O)N(CC(C)C)CC(C)C)S(C)(=O)=O)nn1C] | Cmpd. B 99 |
| 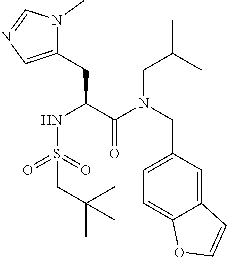 [SMILES: CC(C)CN(Cc1ccc2occc2c1)C(=O)[C@H](Cc1cncn1C)NS(=O)(=O)CC(C)(C)C] | Cmpd. B 100 |
| 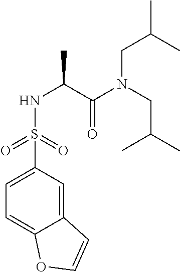 [SMILES: CC(C)CN(CC(C)C)C(=O)[C@H](C)NS(=O)(=O)c1ccc2occc2c1] | Cmpd. B 101 |
| 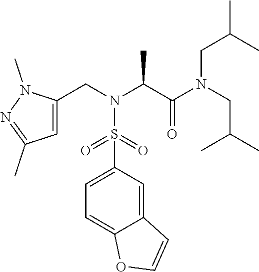 [SMILES: Cc1cc(CN([C@@H](C)C(=O)N(CC(C)C)CC(C)C)S(=O)(=O)c2ccc3occc3c2)n(C)n1] | Cmpd. B 102 |
| 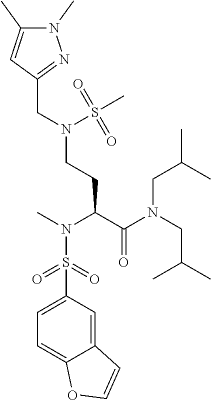 [SMILES: Cc1cc(CN(CC[C@@H](C(=O)N(CC(C)C)CC(C)C)N(C)S(=O)(=O)c2ccc3occc3c2)S(C)(=O)=O)nn1C] | Cmpd. B 103 |
| 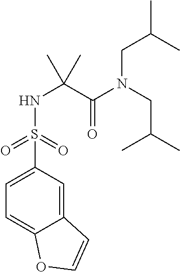 [SMILES: CC(C)CN(CC(C)C)C(=O)C(C)(C)NS(=O)(=O)c1ccc2occc2c1] | Cmpd. B 104 |
| 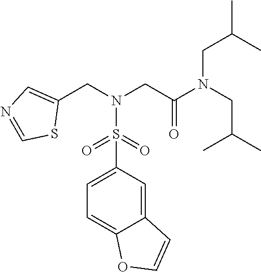 [SMILES: CC(C)CN(CC(C)C)C(=O)CN(Cc1cncs1)S(=O)(=O)c1ccc2occc2c1] | Cmpd. B 105 |
| 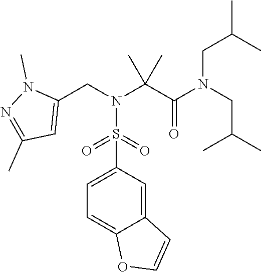 [SMILES: Cc1cc(CN(C(C)(C)C(=O)N(CC(C)C)CC(C)C)S(=O)(=O)c2ccc3occc3c2)n(C)n1] | Cmpd. B 106 |
| 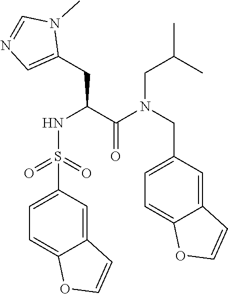 [SMILES: CC(C)CN(Cc1ccc2occc2c1)C(=O)[C@H](Cc1cncn1C)NS(=O)(=O)c1ccc2occc2c1] | Cmpd. B 107 |
| 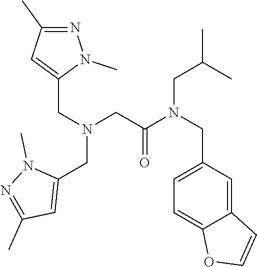 [SMILES: Cc1cc(CN(CC(=O)N(Cc2ccc3occc3c2)CC(C)C)Cc2cc(C)nn2C)n(C)n1] | Cmpd. B 108 |
| 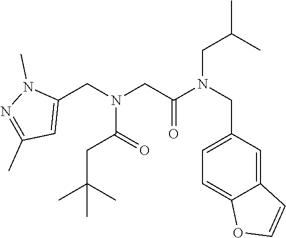 [SMILES: Cc1cc(CN(CC(=O)N(Cc2ccc3occc3c2)CC(C)C)C(=O)CC(C)(C)C)n(C)n1] | Cmpd. B 109 |
| 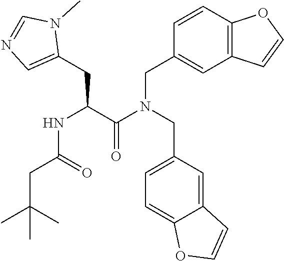 [SMILES: Cn1cncc1C[C@H](NC(=O)CC(C)(C)C)C(=O)N(Cc1ccc2occc2c1)Cc1ccc2occc2c1] | Cmpd. B 110 |
| 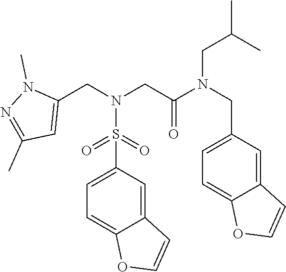 [SMILES: Cc1cc(CN(CC(=O)N(Cc2ccc3occc3c2)CC(C)C)S(=O)(=O)c2ccc3occc3c2)n(C)n1] | Cmpd. B 111 |
| 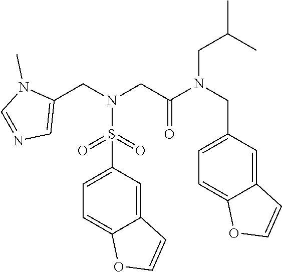 [SMILES: CC(C)CN(Cc1ccc2occc2c1)C(=O)CN(Cc1cncn1C)S(=O)(=O)c1ccc2occc2c1] | Cmpd. B 112 |
| 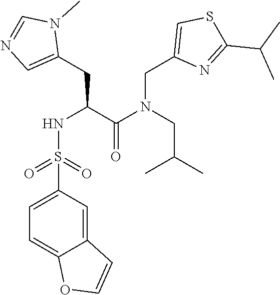 [SMILES: CC(C)CN(Cc1csc(C(C)C)n1)C(=O)[C@H](Cc1cncn1C)NS(=O)(=O)c1ccc2occc2c1] | Cmpd. B 113 |
| 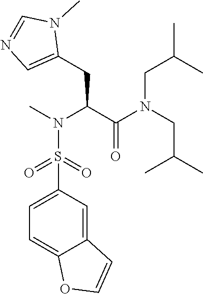 [SMILES: CC(C)CN(CC(C)C)C(=O)[C@H](Cc1cncn1C)N(C)S(=O)(=O)c1ccc2occc2c1] | Cmpd. B 114 |
| 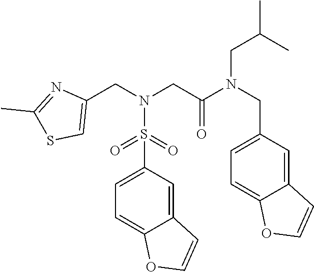 [SMILES: Cc1nc(CN(CC(=O)N(Cc2ccc3occc3c2)CC(C)C)S(=O)(=O)c2ccc3occc3c2)cs1] | Cmpd. B 115 |
| 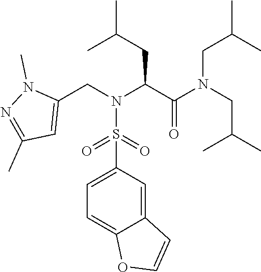 [SMILES: Cc1cc(CN([C@@H](CC(C)C)C(=O)N(CC(C)C)CC(C)C)S(=O)(=O)c2ccc3occc3c2)n(C)n1] | Cmpd. B 116 |
| 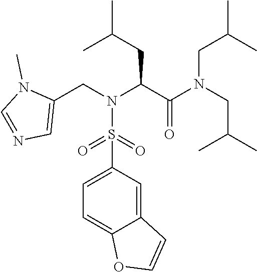 [SMILES: CC(C)C[C@@H](C(=O)N(CC(C)C)CC(C)C)N(Cc1cncn1C)S(=O)(=O)c1ccc2occc2c1] | Cmpd. B 117 |
| 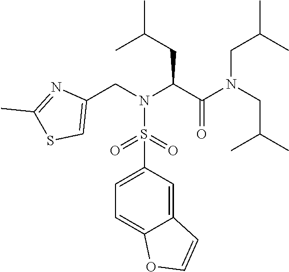 [SMILES: Cc1nc(CN([C@@H](CC(C)C)C(=O)N(CC(C)C)CC(C)C)S(=O)(=O)c2ccc3occc3c2)cs1] | Cmpd. B 118 |
| 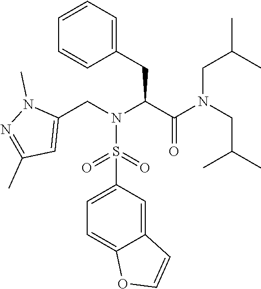 [SMILES: Cc1cc(CN([C@@H](Cc2ccccc2)C(=O)N(CC(C)C)CC(C)C)S(=O)(=O)c2ccc3occc3c2)n(C)n1] | Cmpd. B 119 |
| 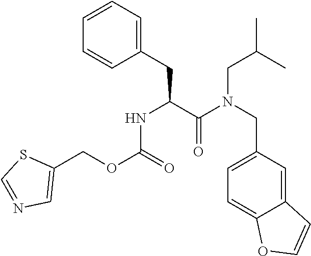 [SMILES: CC(C)CN(Cc1ccc2occc2c1)C(=O)[C@H](Cc1ccccc1)NC(=O)OCc1cncs1] | Cmpd. B 120 |
| 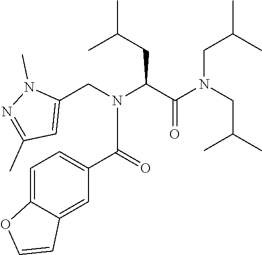 [SMILES: Cc1cc(CN(C(=O)c2ccc3occc3c2)[C@@H](CC(C)C)C(=O)N(CC(C)C)CC(C)C)n(C)n1] | Cmpd. B 121 |
| 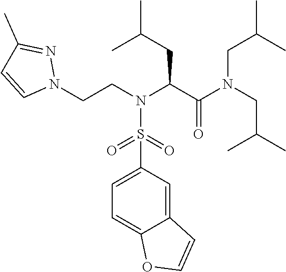 [SMILES: Cc1ccn(CCN([C@@H](CC(C)C)C(=O)N(CC(C)C)CC(C)C)S(=O)(=O)c2ccc3occc3c2)n1] | Cmpd. B 122 |
| 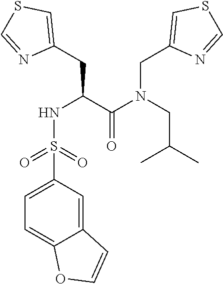 [SMILES: CC(C)CN(Cc1cscn1)C(=O)[C@H](Cc1cscn1)NS(=O)(=O)c1ccc2occc2c1] | Cmpd. B 123 |
| 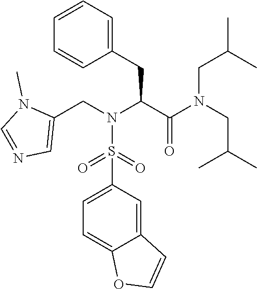 [SMILES: CC(C)CN(CC(C)C)C(=O)[C@H](Cc1ccccc1)N(Cc1cncn1C)S(=O)(=O)c1ccc2occc2c1] | Cmpd. B 124 |
| 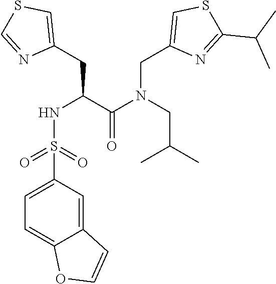 [SMILES: CC(C)CN(Cc1csc(C(C)C)n1)C(=O)[C@H](Cc1cscn1)NS(=O)(=O)c1ccc2occc2c1] | Cmpd. B 125 |
| 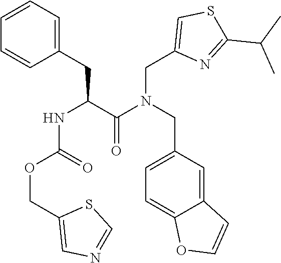 [SMILES: CC(C)c1nc(CN(Cc2ccc3occc3c2)C(=O)[C@H](Cc2ccccc2)NC(=O)OCc2cncs2)cs1] | Cmpd. B 126 |
| 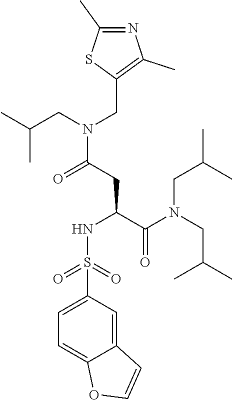 [SMILES: Cc1nc(C)c(CN(CC(C)C)C(=O)C[C@H](NS(=O)(=O)c2ccc3occc3c2)C(=O)N(CC(C)C)CC(C)C)s1] | Cmpd. B 127 |
| 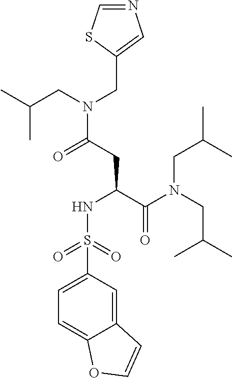 [SMILES: CC(C)CN(Cc1cncs1)C(=O)C[C@H](NS(=O)(=O)c1ccc2occc2c1)C(=O)N(CC(C)C)CC(C)C] | Cmpd. B 128 |
| 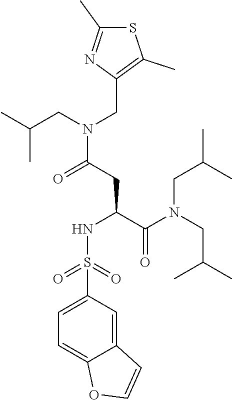 [SMILES: Cc1nc(CN(CC(C)C)C(=O)C[C@H](NS(=O)(=O)c2ccc3occc3c2)C(=O)N(CC(C)C)CC(C)C)c(C)s1] | Cmpd. B 129 |
| 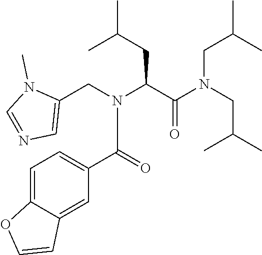 [SMILES: CC(C)C[C@@H](C(=O)N(CC(C)C)CC(C)C)N(Cc1cncn1C)C(=O)c1ccc2occc2c1] | Cmpd. B 130 |
| 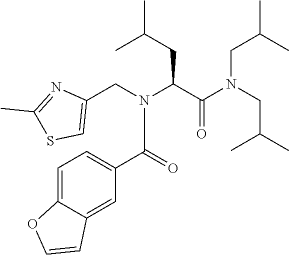 [SMILES: Cc1nc(CN(C(=O)c2ccc3occc3c2)[C@@H](CC(C)C)C(=O)N(CC(C)C)CC(C)C)cs1] | Cmpd. B 131 |
| 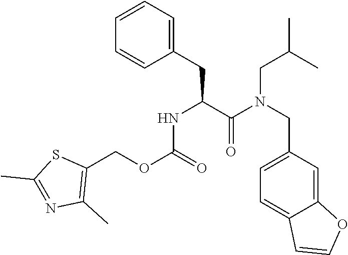 [SMILES: Cc1nc(C)c(COC(=O)N[C@@H](Cc2ccccc2)C(=O)N(Cc2ccc3ccoc3c2)CC(C)C)s1] | Cmpd. B 132 |
| 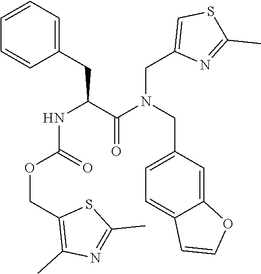 [SMILES: Cc1nc(CN(Cc2ccc3ccoc3c2)C(=O)[C@H](Cc2ccccc2)NC(=O)OCc2sc(C)nc2C)cs1] | Cmpd. B 133 |
| 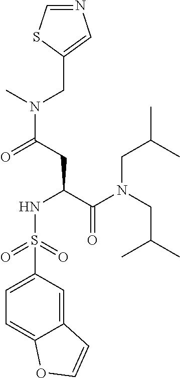 [SMILES: CC(C)CN(CC(C)C)C(=O)[C@H](CC(=O)N(C)Cc1cncs1)NS(=O)(=O)c1ccc2occc2c1] | Cmpd. B 134 |
| 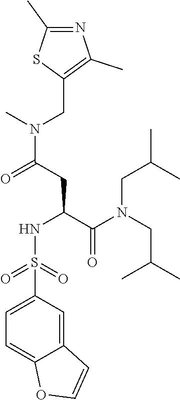 [SMILES: Cc1nc(C)c(CN(C)C(=O)C[C@H](NS(=O)(=O)c2ccc3occc3c2)C(=O)N(CC(C)C)CC(C)C)s1] | Cmpd. B 135 |
| 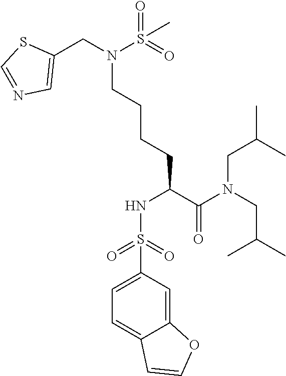 [SMILES: CC(C)CN(CC(C)C)C(=O)[C@H](CCCCN(Cc1cncs1)S(C)(=O)=O)NS(=O)(=O)c1ccc2ccoc2c1] | Cmpd. B 136 |
| 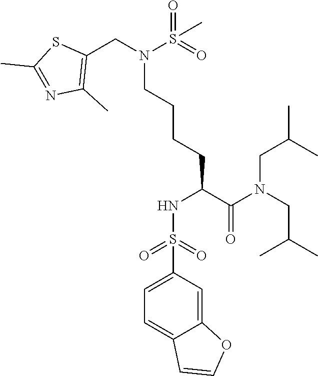 [SMILES: Cc1nc(C)c(CN(CCCC[C@H](NS(=O)(=O)c2ccc3ccoc3c2)C(=O)N(CC(C)C)CC(C)C)S(C)(=O)=O)s1] | Cmpd. B 137 |
| 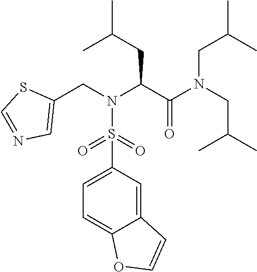 [SMILES: CC(C)C[C@@H](C(=O)N(CC(C)C)CC(C)C)N(Cc1cncs1)S(=O)(=O)c1ccc2occc2c1] | Cmpd. B 138 |
| 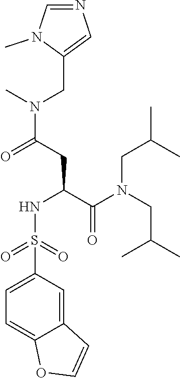 [SMILES: CC(C)CN(CC(C)C)C(=O)[C@H](CC(=O)N(C)Cc1cncn1C)NS(=O)(=O)c1ccc2occc2c1] | Cmpd. B 139 |
| 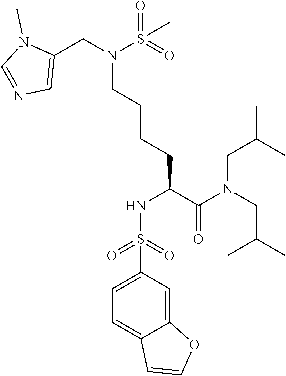 [SMILES: CC(C)CN(CC(C)C)C(=O)[C@H](CCCCN(Cc1cncn1C)S(C)(=O)=O)NS(=O)(=O)c1ccc2ccoc2c1] | Cmpd. B 140 |
| 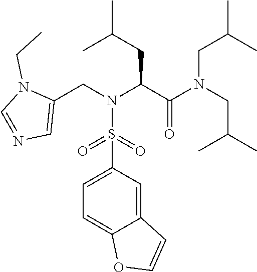 [SMILES: CCn1cncc1CN([C@@H](CC(C)C)C(=O)N(CC(C)C)CC(C)C)S(=O)(=O)c1ccc2occc2c1] | Cmpd. B 141 |
| 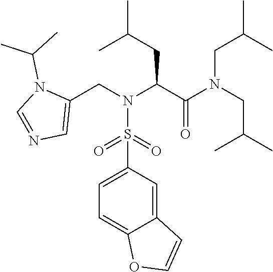 [SMILES: CC(C)C[C@@H](C(=O)N(CC(C)C)CC(C)C)N(Cc1cncn1C(C)C)S(=O)(=O)c1ccc2occc2c1] | Cmpd. B 142 |
| 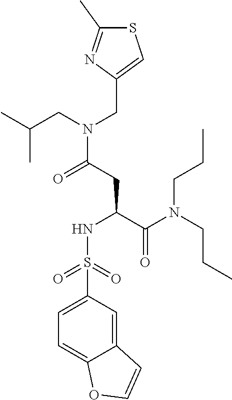 [SMILES: CCCN(CCC)C(=O)[C@H](CC(=O)N(Cc1csc(C)n1)CC(C)C)NS(=O)(=O)c1ccc2occc2c1] | Cmpd. B 143 |
| 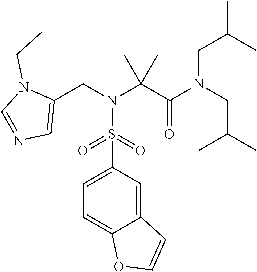 [SMILES: CCn1cncc1CN(C(C)(C)C(=O)N(CC(C)C)CC(C)C)S(=O)(=O)c1ccc2occc2c1] | Cmpd. B 144 |
| 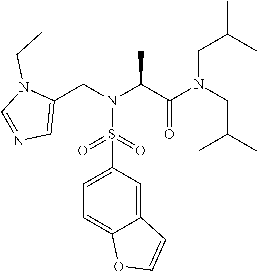 [SMILES: CCn1cncc1CN([C@@H](C)C(=O)N(CC(C)C)CC(C)C)S(=O)(=O)c1ccc2occc2c1] | Cmpd. B 145 |
| 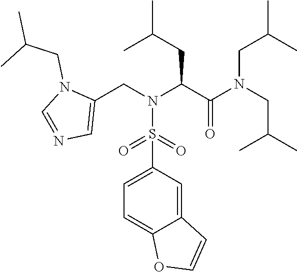 [SMILES: CC(C)C[C@@H](C(=O)N(CC(C)C)CC(C)C)N(Cc1cncn1CC(C)C)S(=O)(=O)c1ccc2occc2c1] | Cmpd. B 146 |
| 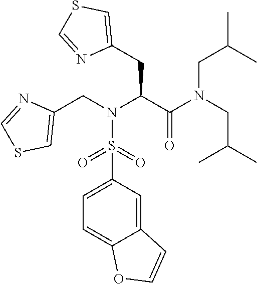 [SMILES: CC(C)CN(CC(C)C)C(=O)[C@H](Cc1cscn1)N(Cc1cscn1)S(=O)(=O)c1ccc2occc2c1] | Cmpd. B 147 |
| 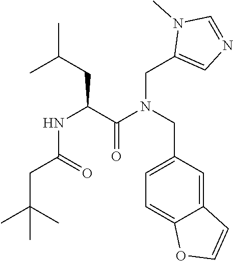 [SMILES: CC(C)C[C@H](NC(=O)CC(C)(C)C)C(=O)N(Cc1ccc2occc2c1)Cc1cncn1C] | Cmpd. B 148 |
| 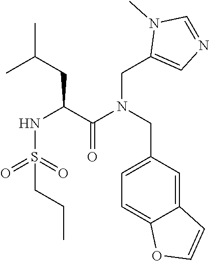 [SMILES: CCCS(=O)(=O)N[C@@H](CC(C)C)C(=O)N(Cc1ccc2occc2c1)Cc1cncn1C] | Cmpd. B 149 |
| 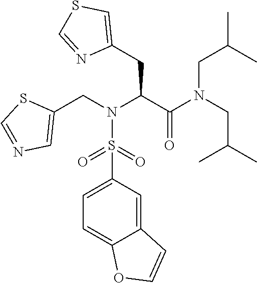 [SMILES: CC(C)CN(CC(C)C)C(=O)[C@H](Cc1cscn1)N(Cc1cncs1)S(=O)(=O)c1ccc2occc2c1] | Cmpd. B 150 |
| 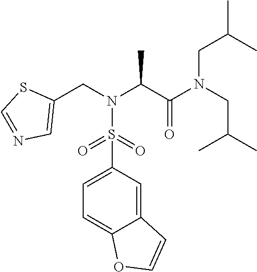 [SMILES: CC(C)CN(CC(C)C)C(=O)[C@H](C)N(Cc1cncs1)S(=O)(=O)c1ccc2occc2c1] | Cmpd. B 151 |
| 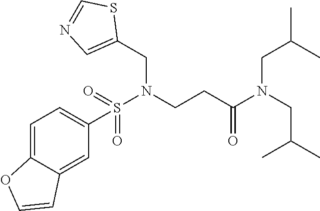 [SMILES: CC(C)CN(CC(C)C)C(=O)CCN(Cc1cncs1)S(=O)(=O)c1ccc2occc2c1] | Cmpd. B 152 |
| 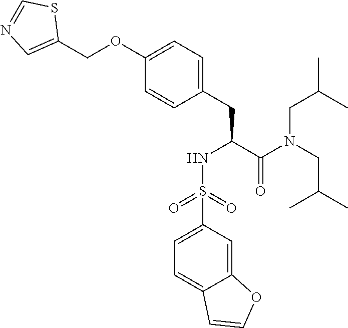 [SMILES: CC(C)CN(CC(C)C)C(=O)[C@H](Cc1ccc(OCc2cncs2)cc1)NS(=O)(=O)c1ccc2ccoc2c1] | Cmpd. B 153 |
| 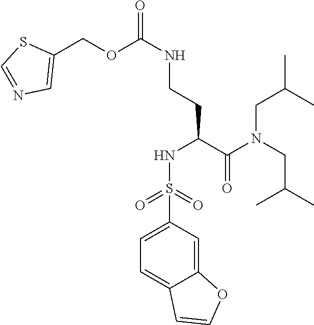 [SMILES: CC(C)CN(CC(C)C)C(=O)[C@H](CCNC(=O)OCc1cncs1)NS(=O)(=O)c1ccc2ccoc2c1] | Cmpd. B 154 |
| 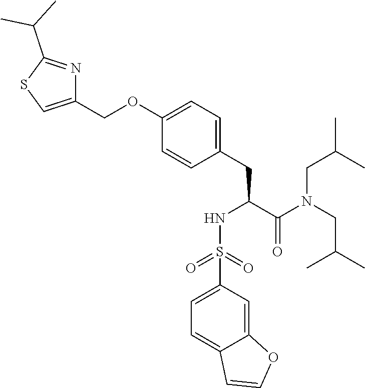 [SMILES: CC(C)CN(CC(C)C)C(=O)[C@H](Cc1ccc(OCc2csc(C(C)C)n2)cc1)NS(=O)(=O)c1ccc2ccoc2c1] | Cmpd. B 155 |
| 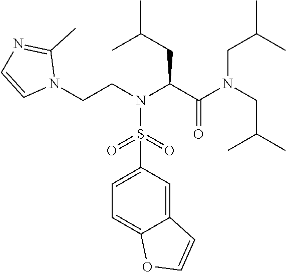 [SMILES: Cc1nccn1CCN([C@@H](CC(C)C)C(=O)N(CC(C)C)CC(C)C)S(=O)(=O)c1ccc2occc2c1] | Cmpd. B 156 |
| 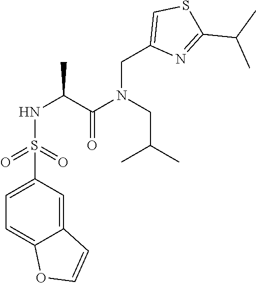 [SMILES: CC(C)CN(Cc1csc(C(C)C)n1)C(=O)[C@H](C)NS(=O)(=O)c1ccc2occc2c1] | Cmpd. B 154 |
| 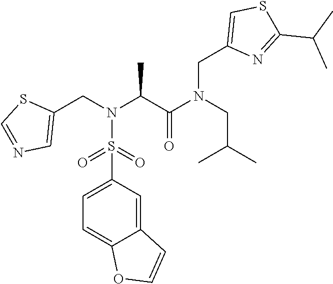 [SMILES: CC(C)CN(Cc1csc(C(C)C)n1)C(=O)[C@H](C)N(Cc1cncs1)S(=O)(=O)c1ccc2occc2c1] | Cmpd. B 158 |
| 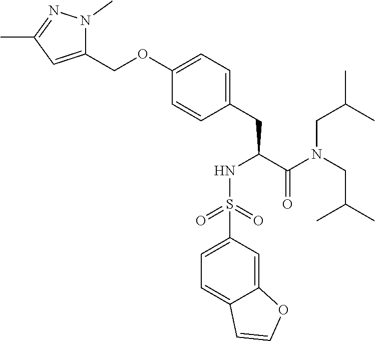 [SMILES: Cc1cc(COc2ccc(C[C@H](NS(=O)(=O)c3ccc4ccoc4c3)C(=O)N(CC(C)C)CC(C)C)cc2)n(C)n1] | Cmpd. B 159 |
| 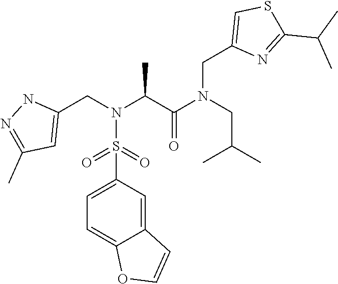 [SMILES: Cc1cc(CN([C@@H](C)C(=O)N(Cc2csc(C(C)C)n2)CC(C)C)S(=O)(=O)c2ccc3occc3c2)[nH]n1] | Cmpd. B 160 |
| 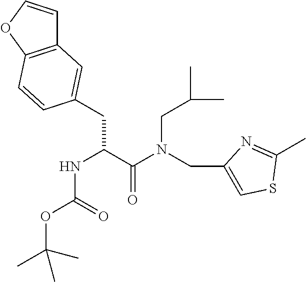 [SMILES: Cc1nc(CN(CC(C)C)C(=O)[C@@H](Cc2ccc3occc3c2)NC(=O)OC(C)(C)C)cs1] | Cmpd. B 161 |
| 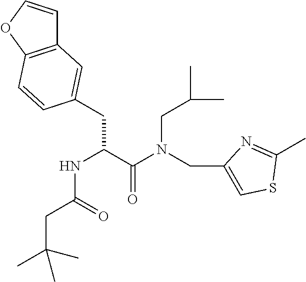 [SMILES: Cc1nc(CN(CC(C)C)C(=O)[C@@H](Cc2ccc3occc3c2)NC(=O)CC(C)(C)C)cs1] | Cmpd. B 162 |
| 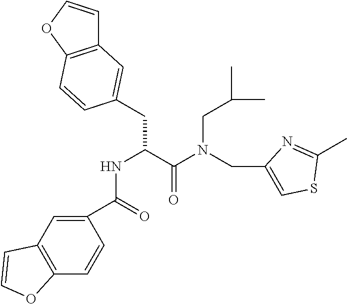 [SMILES: Cc1nc(CN(CC(C)C)C(=O)[C@@H](Cc2ccc3occc3c2)NC(=O)c2ccc3occc3c2)cs1] | Cmpd. B 163 |
| 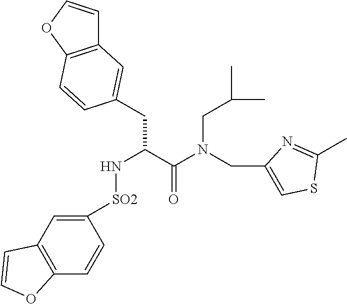 [SMILES: Cc1nc(CN(CC(C)C)C(=O)[C@@H](Cc2ccc3occc3c2)NS(=O)(=O)c2ccc3occc3c2)cs1] | Cmpd. B 164 |
| 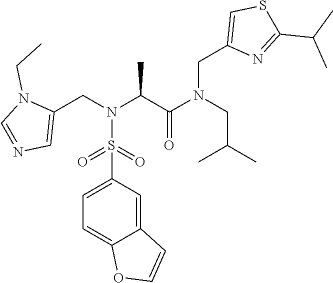 [SMILES: CCn1cncc1CN([C@@H](C)C(=O)N(Cc1csc(C(C)C)n1)CC(C)C)S(=O)(=O)c1ccc2occc2c1] | Cmpd. B 165 |
| 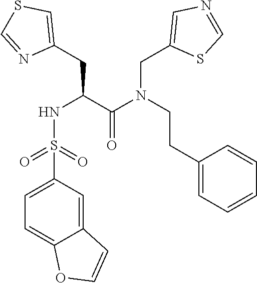 [SMILES: O=C([C@H](Cc1cscn1)NS(=O)(=O)c1ccc2occc2c1)N(CCc1ccccc1)Cc1cncs1] | Cmpd. B 166 |
| 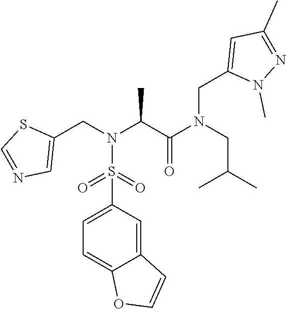 [SMILES: Cc1cc(CN(CC(C)C)C(=O)[C@H](C)N(Cc2cncs2)S(=O)(=O)c2ccc3occc3c2)n(C)n1] | Cmpd. B 167 |
| 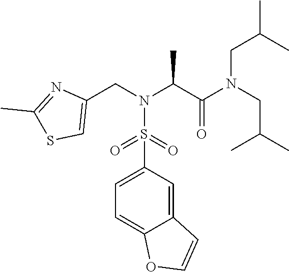 [SMILES: Cc1nc(CN([C@@H](C)C(=O)N(CC(C)C)CC(C)C)S(=O)(=O)c2ccc3occc3c2)cs1] | Cmpd. B 168 |
| 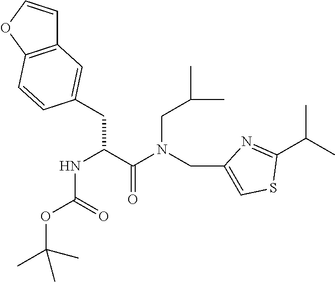 [SMILES: CC(C)CN(Cc1csc(C(C)C)n1)C(=O)[C@@H](Cc1ccc2occc2c1)NC(=O)OC(C)(C)C] | Cmpd. B 169 |
| 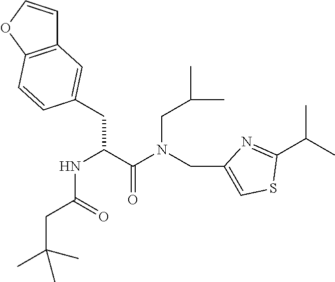 [SMILES: CC(C)CN(Cc1csc(C(C)C)n1)C(=O)[C@@H](Cc1ccc2occc2c1)NC(=O)CC(C)(C)C] | Cmpd. B 170 |
| 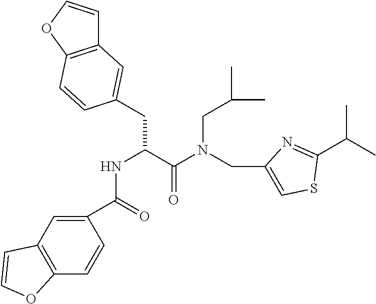 [SMILES: CC(C)CN(Cc1csc(C(C)C)n1)C(=O)[C@@H](Cc1ccc2occc2c1)NC(=O)c1ccc2occc2c1] | Cmpd. B 171 |
| 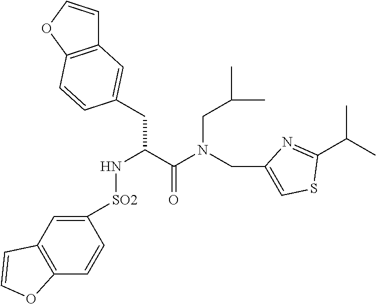 [SMILES: CC(C)CN(Cc1csc(C(C)C)n1)C(=O)[C@@H](Cc1ccc2occc2c1)NS(=O)(=O)c1ccc2occc2c1] | Cmpd. B 172 |
| 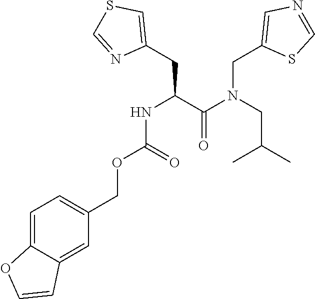 [SMILES: CC(C)CN(Cc1cncs1)C(=O)[C@H](Cc1cscn1)NC(=O)OCc1ccc2occc2c1] | Cmpd. B 173 |
| 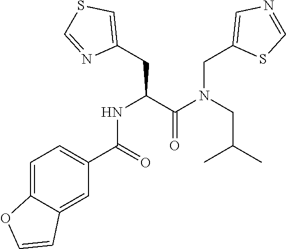 [SMILES: CC(C)CN(Cc1cncs1)C(=O)[C@H](Cc1cscn1)NC(=O)c1ccc2occc2c1] | Cmpd. B 174 |
| 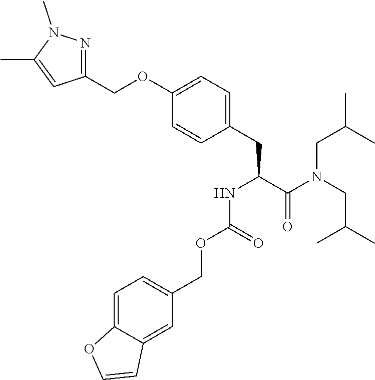 [SMILES: Cc1cc(COc2ccc(C[C@H](NC(=O)OCc3ccc4occc4c3)C(=O)N(CC(C)C)CC(C)C)cc2)nn1C] | Cmpd. B 175 |
| 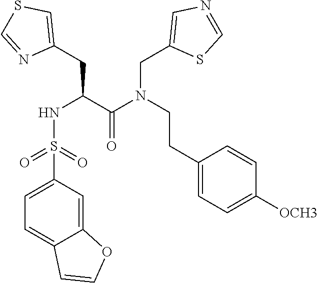 [SMILES: COc1ccc(CCN(Cc2cncs2)C(=O)[C@H](Cc2cscn2)NS(=O)(=O)c2ccc3ccoc3c2)cc1] | Cmpd. B 176 |
| 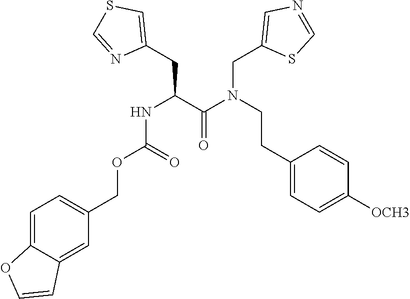 [SMILES: COc1ccc(CCN(Cc2cncs2)C(=O)[C@H](Cc2cscn2)NC(=O)OCc2ccc3occc3c2)cc1] | Cmpd. B 177 |
| 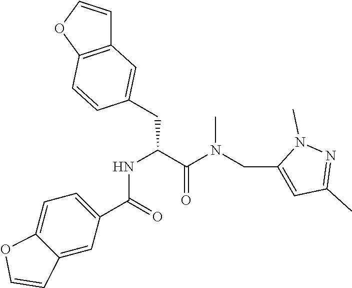 [SMILES: Cc1cc(CN(C)C(=O)[C@@H](Cc2ccc3occc3c2)NC(=O)c2ccc3occc3c2)n(C)n1] | Cmpd. B 178 |

In other embodiments, the ICI does not inhibit HIV protease. In the context of the present invention, a compound is said to not inhibit HIV protease when the Ki of the compound is greater than about 1 μM. Such a Ki means that the compound is not clinically useful for inhibiting HIV protease in a patient infected with HIV.

In general, in the context of an ICI when administered as described herein the terms “effective amount,” “pharmaceutically effective amount,” “therapeutically effective amount” or “therapeutic dose” or “efficacious dose” refer to an amount that when administered to a subject is effective in inhibiting a CYP enough to reduce or prevent the in vivo degradation, and more specifically the intracellular CYP-mediated degradation of an anticancer drug in cancer cells, thereby boosting the drug's efficacy. As used herein, a “subject” refers to a mammal, including a human.

The term “lipophilic group” as used herein refers to a group that, when a part of a compound, increases the affinity or propensity of the compound to bind, attach or dissolve in fat, lipid or oil rather than water. A measure of the lipophilicity or hydrophobicity of compounds of the technology can be calculated using the Hansch equation:

Log 1/C=kP

where C is the concentration of a compound in a given solvent and P is the hydrophobicity. Details of this method can be obtained from J. Amer. Chem. Soc, 86:5175 (1964) and DRUG DESIGN I, edited by E. J. Ariens, Academic Press (1971), both of which are hereby incorporated by reference in their entireties.

Examples of a typical lipophilic group include, but are not limited to, alkyl groups such as methyl, ethyl, n-propyl, isopropyl, n-butyl, isobutyl, t-butyl, n-pentyl, isopentyl, neopentyl, amyl, n-hexyl, n-heptyl, cyclohexyl, cycloheptyl, octyl, nonyl, decyl, undecyl, and dodecyl, alkenes such as ethylene, propylene, butene, pentene, hexene, cyclohexene, heptene, cycloheptene, octene, cyclooctene, nonene, decene, undecene, dodecene, 1,3-butadiene, alkynes such as propyne and butyne, aryls such as phenyl, naphthyl, anthracenyl, phenanthrenyl, fluorenyl, aralkyls such as benzyl, heterocycles such as tetrahydrothiophene, dihydrobenzofuran, heteroaryls such as pyrrole, furan, thiophene, pyrazole, thiazole, indole, carbazole, benzofuran, benzothiophene, indazole, benzothiazole, purine, pyridine, pyridazine, pyrazine, triazine, quinoline, acridine, isoquinoline, and phenanthroline.

For small groups containing heteroatom substituents, such as small heterocycles with a high ratio of heteroatoms to carbon atoms, the introduction of substituents that reduce the heteroatom to carbon atom ratio renders the group lipophilic. For example, a triazole ring can be rendered more lipophilic by the introduction of alkyl substituents. Similarly, non-lipophilic substituents such as hydroxy or amido can be rendered lipophilic by introducing additional carbon atoms, for example by exchanging a hydroxymethyl group to a hydroxybenzyl group, or by exchanging a carboxamido group to a dialkyl carboxamido group.

The term “substituted”, whether preceded by the term “optionally” or not, and substitutions contained in formulas of this technology, include the replacement of one or more hydrogen radicals in a given structure with the radical of a specified substituent. When more than one position in a given structure can be substituted with more than one substituent selected from a specified group, the substituents can be either the same or different at every position (for example, in the moiety —N(R2)(R2), the two R2 substituents can be the same or different). Typically, when a structure can be optionally substituted, 0-3 substitutions are preferred, and 0-1 substitution is more preferred. Advantageously, each substituent enhances cytochrome P450 inhibitory activity in permissive mammalian cells, or enhances deliverability by improving solubility characteristics or pharmacokinetic or pharmacodynamic profiles as compared to the unsubstituted compound. Combinations of substituents and variables envisioned by this technology are limited to those that result in the formation of stable compounds.

The term “stable”, as used herein, refers to compounds which possess stability sufficient to allow manufacture, formulation, and administration to a mammal by methods known in the art. Typically, such compounds are stable at a temperature of 40° C. or less, in the absence of moisture or other chemically reactive conditions, for at least a week.

The term “alkyl”, alone or in combination with any other term, refers to a straight-chain or branched-chain saturated aliphatic hydrocarbon radical containing the specified number of carbon atoms, or where no number is specified, advantageously from 1 to about 12 or 1 to 15 carbon atoms. Examples of alkyl radicals include, but are not limited to: methyl, ethyl, n-propyl, isopropyl, n-butyl, isobutyl, sec-butyl, tert-butyl, pentyl, isoamyl, n-hexyl and the like.

The term “alkenyl”, alone or in combination with any other term, refers to a straight-chain or branched-chain mono- or poly-unsaturated aliphatic hydrocarbon radical containing the specified number of carbon atoms, or where no number is specified, advantageously from 2-6 or 2-10 carbon atoms. Alkenyl groups include all possible E and Z isomers unless specifically stated otherwise. Examples of alkenyl radicals include, but are not limited to, ethenyl, propenyl, isopropenyl, butenyl, isobutenyl, pentenyl, hexenyl, hexadienyl and the like.

The term “alkynyl,” alone or in combination with any other term, refers to a straight-chain or branched-chain hydrocarbon radical having one or more triple bonds containing the specified number of carbon atoms, or where no number is specified, advantageously from 2 to about 10 carbon atoms. Examples of alkynyl radicals include, but are not limited to, ethynyl, propynyl, propargyl, butynyl, pentynyl and the like.

The term “alkoxy” refers to an alkyl ether radical, where the term “alkyl” is as defined above. Examples of suitable alkyl ether radicals include, but are not limited to, methoxy, ethoxy, n-propoxy, isopropoxy, n-butoxy, isobutoxy, sec-butoxy, tert-butoxy and the like.

The terms “alkylamino” or “dialkylamino” include amino radicals substituted by one or two alkyl groups, where the term “alkyl” is defined above, and the alkyl group can be the same or different. Examples of suitable alkylamino and dialkylamino radicals include, but are not limited to, methylamino, ethylamino, isopropylamino, dimethylamino, methylethylamino, ethylbutylamino and the like.

The term “hydroxyalkyl” refers to an alkyl radical as defined above in which one of the hydrogen atoms is replaced by hydroxy group. Examples of suitable hydroxyalkyl radicals include, but are not limited to, hydroxymethyl, 2-hydroxypropyl and the like.

The term “alkoxyalkyl” refers to an alkyl radical as defined above in which one of the hydrogen atoms is replaced by an alkoxy radical as defined above.

The terms “aminoalkyl”, “alkylaminoalkyl” or “dialkylaminoalkyl” refers to an alkyl radical as defined above in which one of the hydrogen atoms is replaced by an amino or “alkylamino” or “dialkylamino” radical as defined above.

The term “halo” or “halogen” includes fluorine, chlorine, bromine or iodine.

The term “haloalkyl” includes alkyl groups with one or more of its hydrogens replaced by halogens.

The term “thioalkyl” includes alkyl radicals having at least one sulfur atom, where alkyl has the significance given above. An example of a thioalkyl is CH₃SCH₂. The definition also encompasses the corresponding sulfoxide and sulfone of this thioalkyl CH₃S(O)CH₂ and CH₃S(O)₂CH₂ respectively. Unless expressly stated to the contrary, the terms “—SO₂—” and “—S(O)₂—” as used herein include sulfone or sulfone derivative (i.e., both appended groups linked to the S), and not a sulfinate ester.

The terms “carboalkoxy” or “alkoxycarbonyl” include alkyl esters of a carboxylic acid. Examples of “carboalkoxy” or “alkoxycarbonyl” radicals include, but are not limited to ethoxycarbonyl (or carboethoxy), Boc (or t-butoxycarbonyl), Cbz (or benzyloxycarbonyl) and the like.

The term “alkanoyl” includes acyl radicals derived from an alkanecarboxylic acid. Examples of alkanoyl radicals include, but are not limited to acetyl, propionyl, isobutyryl and the like.

The term “aryl,” alone or in combination with any other term, refers to a carbocyclic aromatic radical (such as phenyl or naphthyl) containing the specified number of carbon atoms, preferably from 6-15 carbon atoms, and more preferably from 6-10 carbon atoms, optionally substituted with one or more substituents selected from alkyl, alkoxy, (for example methoxy), nitro, halo, amino, mono or dialkylamino, carboalkoxy, cyano, thioalkyl, alkanoyl, carboxylate, and hydroxy. Examples of aryl radicals include, but are not limited to phenyl, p-tolyl, 4-hydroxyphenyl, 1-naphthyl, 2-naphthyl, indenyl, indanyl, azulenyl, fluorenyl, anthracenyl and the like.

The term “aralkyl”, alone or in combination, includes alkyl radicals as defined above in which one or more hydrogen atoms is replaced by an aryl radical as defined above. Examples of aralkyl radicals include, but are not limited to benzyl, 2-phenylethyl and the like.

The term “aralkanoyl” includes acyl radicals derived from an aryl-substituted alkanecarboxylic acid such as phenylacetyl, 3-phenylpropionyl (hydrocinnamoyl), 4-phenylbutyryl, (2-naphthyl)acetyl, 4-chlorohydrocinnamoyl, 4-aminohydrocinnamoyl, (1-naphthyl)acetyl, 4-methoxyhydrocinnamoyl, and the like.

The term “aroyl” includes acyl radicals derived from an aromatic carboxylic acid such as benzoyl, 4-chlorobenzoyl, 4-carboxybenzoyl, 4-benzyloxycarbonyl)benzoyl, 1-naphthoyl, 2-naphthoyl, 6-carboxy-2-naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The term “arylsulfonyl” includes sulfonyl radicals derived from an aromatic sulfonic acid such as benzenesulfonyl, 4-chlorobenzenesulfonyl, 1-naphthalenesulfonyl, 2-naphthalenesulfonyl, and the like.

The term “carbocycle” refers to a non-aromatic stable 3- to 8-membered carbon ring which can be saturated, mono-unsaturated or poly-unsaturated. The carbocycle can be attached at any endocyclic carbon atom which results in a stable structure. Preferred carbocycles have 5-7 carbons.

The term “cycloalkyl”, alone or in combination, includes alkyl radicals which contain from about 3 to about 8 carbon atoms and are cyclic. Examples of such cycloalkyl radicals include cyclopropyl, cyclobutyl, cyclopentyl, cyclohexyl and the like.

The term “cycloalkenyl” alone or in combination includes alkenyl radicals as defined above which contain about 3-8 carbon atoms and are cyclic.

The term “cycloalkylalkyl” includes alkyl radicals as defined above which are substituted by a cycloalkyl radical containing from about 3 to about 8, preferably from about 3 to about 6, carbon atoms.

The term “heterocyclyl” or “heterocyclo” or “heterocycloalkyl” refers to a stable 3-7 membered monocyclic heterocycle or 8-11 membered bicyclic heterocycle which is either saturated or partially unsaturated, and which can be optionally benzofused if monocyclic and which is optionally substituted on one or more carbon atoms by halogen, alkyl, alkoxy, oxo, and the like, and/or on a secondary nitrogen atom (i.e., —NH—) by alkyl, aralkoxycarbonyl, alkanoyl, phenyl or phenylalkyl or on a tertiary nitrogen atom (i.e., +N—) by oxido and which is attached via a carbon atom. Each heterocycle consists of one or more carbon atoms and from one to four heteroatoms selected from the group consisting of nitrogen, oxygen and sulfur. As used herein, the terms “nitrogen and sulfur heteroatoms” include oxidized forms of nitrogen and sulfur, and the quaternized form of any basic nitrogen. A heterocyclyl radical can be attached at any endocyclic carbon or heteroatom which results in the creation of a stable structure. Preferred heterocycles include 5-7 membered monocyclic heterocycles, and 8-10 membered bicyclic heterocycles. Examples of such groups imidazolinyl, imidazolidinyl, indazolinyl, perhydropyridazyl, pyrrolinyl, pyrrolidinyl, piperidinyl, pyrazolinyl, piperazinyl, morpholinyl, thiamorpholinyl, thiazolidinyl, thiamorpholinyl sulfone, oxopiperidinyl, oxopyrrolidinyl, oxoazepinyl, tetrahydropyranyl, tetrahydrofuranyl, dioxolyl, dioxinyl, benzodioxolyl, dithiolyl, tetrahydrothienyl, sulfolanyl, dioxanyl, dioxolanyl, tetahydrofurodihydrofuranyl, tetrahydropyranodihydrofuranyl, dihydropyranyl, tetrahydrofurofuranyl and tetrahydropyranofuranyl.

The term “heteroaryl” refers to stable 5-6 membered monocyclic or 8-11 membered bicyclic or 13-16 membered tricyclic aromatic heterocycles where heterocycles is as defined above. Non-limiting examples of such groups include imidazolyl, quinolyl, isoquinolyl, indolyl, indazolyl, pyridazyl, pyridyl, pyrrolyl, pyrazolyl, pyrazinyl, quinoxolyl, pyranyl, pyrimidinyl, furyl, thienyl, triazolyl, thiazolyl, carbolinyl, tetrazolyl, benzofuranyl, thiamorpholinyl sulfone, oxazolyl, benzoxazolyl, benzimidazolyl, benzthiazolyl, oxopiperidinyl, oxopyrrolidinyl, oxoazepinyl, azepinyl, isoxazolyl, isothiazolyl, furazanyl, thiazolyl, thiadiazyl, oxathiolyl, acridinyl, phenanthridinyl, and benzocinnolinyl.

The term “heterocycloalkylalkyl” refers to an alkyl radical as defined above which is substituted by a heterocycloalkyl radical as defined above.

The term “heteroaralkyl” alone or in combination, includes alkyl radicals as defined above in which one or more hydrogen atom is replaced by a heteroroaryl group as defined above.

The ICIs administered as described herein include pharmaceutically acceptable derivatives or prodrugs thereof. A “pharmaceutically acceptable derivative or prodrug” includes a pharmaceutically acceptable salt, ester, salt of an ester, or other derivative of a compound of this technology which, upon administration to a recipient, is capable of providing (directly or indirectly) a compound of this technology. Particularly favored derivatives and prodrugs are those that increase the bioavailability of the compounds of this technology when such compounds are administered to a mammal (e.g., by allowing an orally administered compound to be more readily absorbed into the blood) or which enhance delivery of the parent compound to a biological compartment (e.g., the brain or lymphatic system) relative to the parent species. Examples of prodrugs of hydroxy containing compounds are amino acid esters or phosphonate or phosphate esters that can be cleaved in vivo hydrolytically or enzymatically to provide the parent compound. These have the advantage of providing potentially improved solubility.

The compounds of this technology can contain one or more asymmetric carbon atoms and thus occur as racemates and racemic mixtures, single enantiomers, diastereomeric mixtures and individual diastereomers. All such isomeric forms of these compounds are expressly included in the technology. Each stereogenic carbon can be of the R or S configuration. Although the specific compounds exemplified in this application can be depicted in a particular stereochemical configuration, compounds having either the opposite stereochemistry at any given chiral center or mixtures thereof are also envisioned.

The ICIs also include compounds with quaternization of any basic nitrogen-containing groups in the compounds. The basic nitrogen can be quaternized with any agents known to those of ordinary skill in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products can be obtained by such quaternization.

Administration of ICIs

The ICI can be co-administered with one or more anticancer drugs, or may be administered substantially contemporaneously with the drug. When the ICI is administered prior to administration of the cancer drug, as described in more detail above, the time period for administering the ICI may be at least 30 minutes, at least 1 hour, at least 2 hours, at least 4 hours, at least 8 hours, or at least 12 hours prior to administration of the drug. In other embodiments the ICI is administered 16, 20, or 24 hours prior to administration of the anticancer drug, or 1, 2, 3, 4, 5, 6, 7, 10 or 14 days prior to administration of the anticancer drug.

The ICIs tend to have a long half in vivo, presumably as a result of inhibiting their own metabolism. This means that once treatment has begun, the ICI may be administered less frequently than the cancer drug, although the skilled artisan will recognize that different administration regiments may be needed in specific situations.

The dosage of the ICI used may depend on the context of the administration but is generally that dose that reduces CYP activity in cancer cells to a level that substantially eliminates CYP-mediated metabolism of the cancer drug in the cancer cells, for a time sufficient for the anticancer drug to be clinically effective. The ICI may be administered orally or parenterally as desired and as determined by the individual circumstances of the patient treatment.

The ICIs can be administered in the form of pharmaceutically acceptable salts derived from inorganic or organic acids. Included among such acid salts, for example, are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate and undecanoate.

Other pharmaceutically acceptable salts include salts with an inorganic base, organic base, inorganic acid, organic acid, or basic or acidic amino acid. Inorganic bases which form the pharmaceutically acceptable salts include alkali metals such as sodium or potassium, alkali earth metals such as calcium and magnesium, aluminum, and ammonia. Organic bases which form pharmaceutically acceptable salts include trimethylamine, triethylamine, pyridine, picoline, ethanolamine, diethanolamine, triethanolamine, dicyclohexylamine.

Inorganic acids which form the pharmaceutically acceptable salts include hydrochloric acid, hydroboric acid, nitric acid, sulfuric acid, and phosphoric acid. Organic acids appropriate to form the salt include formic acid, acetic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid. Basic amino acids to form the salt include arginine, lysine and ornithine. Acidic amino acids to form the salt include aspartic acid and glutamic acid.

The technology also contemplates compositions which can be administered orally or non-orally in the form of, for example, granules, powders, tablets, capsules, syrup, suppositories, injections, emulsions, elixirs, suspensions or solutions, by mixing these effective components, individually or simultaneously, with pharmaceutically acceptable carriers, excipients, binders, diluents or the like.

As a solid formulation for oral administration, the composition can be in the form of powders, granules, tablets, pills and capsules. In these cases, the compounds can be mixed with at least one additive, for example, sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. These formulations can contain, as in conventional cases, further additives, for example, an inactive diluent, a lubricant such as magnesium stearate, a preservative such as paraben or sorbic acid, an anti-oxidant such as ascorbic acid, tocopherol or cysteine, a disintegrator, a binder, a thickening agent, a buffer, a sweetener, a flavoring agent and a perfuming agent. Tablets and pills can further be prepared with enteric coating.

Examples of liquid preparations for oral administration include pharmaceutically acceptable emulsions, syrups, elixirs, suspensions and solutions, which can contain an inactive diluent, for example, water.

As used herein, “non-orally” includes subcutaneous injection, intravenous injection, intramuscular injection, intraperitoneal injection or instillation. Injectable preparations, for example, sterile injectable aqueous suspensions or oil suspensions can be prepared by known procedures in the fields concerned, using a suitable dispersant or wetting agent and suspending agent. The sterile injections can be, for example, a solution or a suspension, which is prepared with a non-toxic diluent administrable non-orally, such as an aqueous solution, or with a solvent employable for sterile injection. Examples of usable vehicles or acceptable solvents include water, Ringer's solution and an isotonic aqueous saline solution. Further, a sterile non-volatile oil can usually be employed as solvent or suspending agent. A non-volatile oil and a fatty acid can be used for this purpose, including natural or synthetic or semi-synthetic fatty acid oil or fatty acid, and natural or synthetic mono- or di- or tri-glycerides.

The pharmaceutical compositions can be formulated for nasal aerosol or inhalation and can be prepared as solutions in saline, and benzyl alcohol or other suitable preservatives, absorption promoters, fluorocarbons, or solubilizing or dispersing agents.

Rectal suppositories can be prepared by mixing the drug with a suitable vehicle, for example, cocoa butter and polyethylene glycol, which is in the solid state at ordinary temperatures, in the liquid state at temperatures in intestinal tubes and melts to release the drug.

The pharmaceutical composition can be easily formulated for topical administration with a suitable ointment containing one or more of the compounds suspended or dissolved in a carrier, which include mineral oil, liquid petroleum, white petroleum, propylene glycol, polyoxyethylene, polyoxypropylene compound, emulsifying wax and water. In addition, topical formulations can be formulated with a lotion or cream containing the active compound suspended or dissolved in a carrier. Suitable carriers include mineral oil, sorbitan monostearate, polysorbate 60, cetyl esters wax, cetaryl alcohol, 2-octyldodecanol, benzyl alcohol and water.

In some embodiments, the pharmaceutical compositions can include α-, β-, or γ-cyclodextrins or their derivatives. In certain embodiments, co-solvents such as alcohols can improve the solubility and/or the stability of the compounds in pharmaceutical compositions. In the preparation of aqueous compositions, addition salts of the compounds can be suitable due to their increased water solubility.

Appropriate cyclodextrins are α-, β-, or γ-cyclodextrins (CDs) or ethers and mixed ethers thereof where one or more of the hydroxy groups of the anhydroglucose units of the cyclodextrin are substituted with C₁-C₆alkyl, such as methyl, ethyl or isopropyl, e.g. randomly methylated β-CD; hydroxy C₁₆ alkyl, particularly hydroxy-ethyl, hydroxypropyl or hydroxybutyl; carboxy C₁-C₆alkyl, particularly carboxymethyl or carboxyethyl; C₁-C₆alkyl-carbonyl, particularly acetyl; C₁-C₆ alkyloxycarbonylC₁-C₆alkyl or carboxyC₁-C₆alkyloxyC₁-C₆alkyl, particularly carboxymethoxypropyl or carboxyethoxypropyl; C₁-C₆alkylcarbonyloxyC₁-C₆alkyl, particularly 2-acetyloxypropyl. Especially noteworthy as complexants and/or solubilizers are β-CD, randomly methylated β-CD, 2,6-dimethyl-β-CD, 2-hydroxyethyl-β-CD, 2-hydroxyethyl-γ-CD, hydroxy-propyl-γ-CD and (2-carboxymethoxy)propyl-β-CD, and in particular 2-hydroxy-propyl-β-CD (2-HP-β-CD).

The term “mixed ether” denotes cyclodextrin derivatives where at least two cyclodextrin hydroxy groups are etherified with different groups such as, for example, hydroxy-propyl and hydroxyethyl.

The compounds can be formulated in combination with a cyclodextrin or a derivative thereof as described in U.S. Pat. No. 5,707,975. Although the formulations described therein are with antifungal active ingredients, they are equally relevant for formulating compounds of the technology. The formulations described therein are particularly suitable for oral administration and comprise an antifungal as active ingredient, a sufficient amount of a cyclodextrin or a derivative thereof as a solubilizer, an aqueous acidic medium as bulk liquid carrier and an alcoholic co-solvent that greatly simplifies the preparation of the composition. The formulations can also be rendered more palatable by adding pharmaceutically acceptable sweeteners and/or flavors.

Other convenient ways to enhance the solubility of the compounds of the technology in pharmaceutical compositions are described in WO 94/05263, WO 98/42318, EP-A-499,299 and WO 97/44014, all incorporated herein by reference.

In some embodiments, the compounds can be formulated in a pharmaceutical composition comprising a therapeutically effective amount of particles consisting of a solid dispersion comprising a compound of formula I, and one or more pharmaceutically acceptable water-soluble polymers.

The term “solid dispersion” defines a system in a solid state comprising at least two components, where one component is dispersed more or less evenly throughout the other component or components. When the dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered.

The term “solid dispersion” also comprises dispersions which are less homogenous throughout than solid solutions. Such dispersions are not chemically and physically uniform throughout or comprise more than one phase.

The water-soluble polymer in the particles is conveniently a polymer that has an apparent viscosity of 1 to 100 mPa·s when dissolved in a 2% aqueous solution at 20° C.

Preferred water-soluble polymers are hydroxypropyl methylcelluloses (HPMC). HPMC having a methoxy degree of substitution from about 0.8 to about 2.5 and a hydroxypropyl molar substitution from about 0.05 to about 3.0 are generally water soluble. Methoxy degree of substitution refers to the average number of methyl ether groups present per anhydroglucose unit of the cellulose molecule. Hydroxypropyl molar substitution refers to the average number of moles of propylene oxide which have reacted with each anhydroglucose unit of the cellulose molecule.

The particles as defined hereinabove can be prepared by first preparing a solid dispersion of the components, and then optionally grinding or milling that dispersion. Various techniques exist for preparing solid dispersions including melt-extrusion, spray-drying and solution-evaporation.

It can further be convenient to formulate the compounds in the form of nanoparticles which have a surface modifier adsorbed on the surface thereof in an amount sufficient to maintain an effective average particle size of less than 1000 nm. Useful surface modifiers are believed to include those which physically adhere to the surface of the antiretroviral agent but do not chemically bond to the antiretroviral agent.

Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants.

The compounds can also be incorporated in hydrophilic polymers and applied as a film over many small beads, thus yielding a composition with good bioavailability which can conveniently be manufactured and which is suitable for preparing pharmaceutical dosage forms for oral administration. The beads comprise a central, rounded or spherical core, a coating film of a hydrophilic polymer and an antiretroviral agent and a seal-coating polymer layer. Materials suitable for use as cores are pharmaceutically acceptable and have appropriate dimensions and firmness. Examples of such materials are polymers, inorganic substances, organic substances, saccharides and derivatives thereof. The route of administration can depend on the condition of the subject, co-medication and the like.

Dosages of the compounds are dependent on age, body weight, general health conditions, sex, diet, dose interval, administration routes, excretion rate, combinations of drugs and conditions of the diseases treated, while taking these and other necessary factors into consideration. Generally, dosage levels of between about 10 μg per day to about 5000 mg per day, preferably between about 100 mg per day to about 1000 mg per day of the compound are useful for the inhibition of CYP enzymes. Typically, the pharmaceutical compositions of this technology will be administered from about 1 to about 3 times per day or alternatively, as a continuous infusion. Such administration can be used as a chronic or acute therapy.

The amount of active ingredient that can be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

While these dosage ranges can be adjusted by a necessary unit base for dividing a daily dose, as described above, such doses are decided depending on the diseases to be treated, conditions of such diseases, the age, body weight, general health conditions, sex, diet of the patient then treated, dose intervals, administration routes, excretion rate, and combinations of drugs, while taking these and other necessary factors into consideration. For example, a typical preparation will contain from about 5% to about 95% active compound (w/w). Preferably, such preparations contain from about 10% to about 80% active compound. The desired unit dose of the composition of this technology is administered once or multiple times daily.

In some embodiments, the technology contemplates compositions and formulations comprising one or more of the compounds in combination with one or more other drugs that can be metabolized or degraded by CYP.

The CYP inhibitors of this technology can be administered to a patient either as a single agent (for use with a separate dose of another drug) or in a combined dosage form with at least one other drug. Additional drugs also can be used to increase the therapeutic effect of these compounds.

Determination of CYP Activity in Cancer Cells

Methods of determining cellular CYP activity are known in the art and can be used to determine whether or not cancer cells express elevated CYP activity. Cancer cells can be obtained by, for example, standard biopsy and tested for CYP activity using histochemistry, p450-GLO assays (Promega Corp., Madison, Wis.), immunoassay and cellular enzyme assay using a fluorescent or colorimetric substrate. The enzyme activity can be measured directly, or the amount of CYP protein or mRNA can be used as a surrogate for CYP activity. The amount of CYP protein can be determined by the methods described above or by, for example, mass spectrometric methods using an isotopically labeled standard. Such methods are well known in the art. A cancer cell expresses an elevated level of CYP activity when the measured CYP activity is significantly higher than the CYP activity in an equivalent non-cancer cell. For example, the levels of CYP activity in a malignant prostate carcinoma cell can be compared to the level in a non-malignant cell from the same patient, or can be compared to reference amounts. An elevated level of CYP activity can be, for example, at least 50%, at least 100%, at least 150%, at least 200%, or at least 300% higher than the activity in the reference cell.

Enhancing Oral Availability of Anticancer Drugs

The ICIs described herein can be used to enhance oral availability of cancer drugs that are systemically metabolized by CYPs. For example, docetaxel is a widely used anticancer drug applied against a variety of cancer types including breast, lung and prostate cancer. A major disadvantage of docetaxel is that it has a very low oral bioavailability and can therefore only be administered intravenously. This low oral bioavailability has been shown to be because the drug is a substrate for CYP3A in the intestinal tract and in the liver, and is also subject to active efflux by the drug transporter MDR1 (P-glycoprotein; ABCB 1) (Miller and Ojima, Chem. Rec. 1(3):195-211, 2001; deWeger et al., Anticancer Drugs 25(5):488-494, 2014). The natural interpatient variability of CYP3A4 activity leads to variable drug exposure and present significant risks of under and overdosing patients with docetaxel (Baker, et al., Clin Pharmacokinet 45:235-52, 2006). Disadvantages of intravenous formulations of docetaxel are severe side effects and dose-limiting toxicities such as neutropenia, hypersensitivity reactions, neurotoxicity, and alopecia. Several new dosage/administration forms are in development, and a more tolerable, albumin-bound, intravenous formulation of paclitaxel (Abraxane) has recently been registered. New formulations of docetaxel are being sought to improve efficacy and safety.

The ICIs described herein can be used to enhance drug oral bioavailability, including that of docetaxel by inhibiting systemic CYP activity, allowing drug exposure to be achieved that is equivalent to, or in the same range as that achieved by intravenous or other parenteral administration.

### Example

Combination of Docataxel and an ICI

The compound of formula IA (150 mg po qd) is administered to patients for the first 3 days of a 21 day treatment cycle. Escalating doses of Docataxel are administered IV beginning on day 4. Docataxel is dose-escalated at 15, 25, 40, and 50 mg/m², where the standard dose is 75 mg/m². In the trial the use of the ICI decreases variability in AUC and drug response, diminishes toxicity (lower dose of docataxel and lower Cₘₐₓ while maintaining AUC) and improves cancer cell kill in tumors expressing high levels of CYP3A4.

1. A method of inhibiting the growth of a tumor in a patient, comprising administering to said patient an effective amount of at least one cytochrome p450 monooxidase inhibitor and an effective amount of an anticancer drug,
wherein said cytochrome p450 monooxidase inhibitor is a functionally irreversible inhibitor,
wherein said effective amount of said cytochrome p450 monooxidase inhibitor is effective to inhibit cytochrome p450 monooxidase activity in said tumor or in cells required for growth of said tumor and to substantially prevent degradation of said anticancer drug in said tumor or in said cells, and wherein said tumor or said cells required for growth of said tumor express elevated levels of at least one cytochrome p450 monooxidase compared to the level in a non-malignant cell from the same patient.

2. The method of claim 1, wherein said effective amount of said cytochrome p450 monooxidase inhibitor is effective to inhibit cytochrome P450 monooxidase activity in said tumor.

3. The method of claim 1, wherein said effective amount of said cytochrome p450 monooxidase inhibitor is effective to inhibit cytochrome P450 monooxidase activity in cells required for growth of said tumor, and wherein said cells are selected from the group consisting of cancer stem cells, stromal cells and endothelial cells.

4. The method of claim 3, wherein said cells are cancer stem cells.

5. The method of claim 3, wherein said cells are stromal cells.

6. The method of claim 3, wherein said cells are endothelial cells.

7. The method of claim 1, wherein said tumor has become resistant to the antitumor activity of at least one anticancer drug.

8. The method of claim 1 wherein said anticancer drug is selected from the group consisting of alkylating agents, vinca alkaloids, aromatase inhibitors, selective estrogen receptor modulators, topoisomerase I inhibitors, topoisomerase II inhibitors, microtubule stabilizing and disrupting agents, tubulin binding agents, tyrosine kinase inhibitors, proteosome inhibitors, mTOR inhibitors and conjugated antibodies.

9. The method of claim 1 wherein said tumor or said cells required for the growth of said tumor have been shown to express elevated levels of said cytochrome p450 monooxidase.

10. A method of preventing or slowing the development of drug resistance in a tumor, comprising administering to a patient suffering from said tumor an effective amount of at least one cytochrome p450 monooxidase inhibitor,
wherein said cytochrome p450 monooxidase inhibitor is an irreversible inhibitor,
wherein said effective amount of said cytochrome p450 monooxidase inhibitor is effective to inhibit cytochrome monooxidase activity in said tumor or in cells required for growth of said tumor and to substantially prevent degradation of a preselected anticancer drug in said tumor or in said cells, and wherein said tumor or said cells required for growth of said tumor express elevated levels of at least one cytochrome p450 monooxidase compared to the level in a non-malignant cell from the same patient.

11. A method of improving a cancer therapy outcome in a patient being treated with at least one anticancer drug, comprising administering to said patient an effective amount of at least one cytochrome p450 monooxidase inhibitor,
wherein said cytochrome p450 monooxidase inhibitor is an irreversible inhibitor,
wherein said effective amount of said cytochrome p450 monooxidase inhibitor is effective to inhibit cytochrome P450 monooxidase activity in tumor cells or in cells required for growth of said tumor cells, and to substantially prevent degradation of a preselected anticancer drug in said tumor cells or in said cells required for growth of said tumor cells, and wherein said tumor or said cells required for growth of said tumor express elevated levels of at least one cytochrome p450 monooxidase compared to the level in a non-malignant cell from the same patient.

12. The method of claim 11, wherein said cancer therapy outcome is selected from the group consisting of improved efficacy, and improved safety.

13. The method of claim 1 wherein said anticancer drug is not substantially degraded by cytochrome activity in systemic circulation outside of said tumor or said cells required for the growth of said tumor.

14. A method of preparing a patient for cancer therapy prior to treatment with an anticancer drug, comprising administering to said patient an effective amount of a cytochrome p450 monooxidase inhibitor,
wherein said cytochrome p450 monooxidase inhibitor is an irreversible inhibitor,
wherein said effective amount of said cytochrome p450 monooxidase inhibitor is effective to inhibit cytochrome P450 monooxidase activity in tumor cells in said patient or in cells in said patient required for growth of said tumor cells such that degradation of said anticancer drug in said tumor or in said cells is substantially inhibited upon subsequent administration of said anticancer drug to said patient, and wherein said tumor or said cells required for growth of said tumor express elevated levels of at least one cytochrome p450 monooxidase compared to the level in a non-malignant cell from the same patient.

15. The method of claim 1, wherein said cytochrome inhibitor is administered to said patient prior to the first treatment with said anticancer drug.

16-17. (canceled)

17. The method of claim 16 wherein the improvement in said trial is reduced interpatient variability of drug-degrading metabolic activity, improved clinical outcome, or reduced clinical trial size.

18. (canceled)

19. The method according claim 1, wherein the anticancer drug or cancer therapy is selected from the group consisting of Cyclophosphamide, Ifosfamide, Vincristine, Vinblastine, Vindesine, Vinorelbine, Exemestane, Letrozole, Tamoxifen, Toremifene, Camptothecin and Camptothecan analogs such as Topotecan And Irinotecan, Etoposide, Teniposide, Taxol and Taxol analogs such as Taxotere, Erlotinib, Lapatanib, Sunitinib, Pazopanib, Imatinib, Dasatanib, Nilotinib, Bortezomib, Temsirolimus, Cyclosporine, Tacrolimus (FK506), Sirolimus (rapamycin), Indinavir, Ritonavir, Saquinavir, Felodipine, Isradipine, Nicardipine, Nisoldipine, Nimodipine, Nitrendipine, Nifedipine, Verapamil, Etoposide, Tamoxifen, Vinblastine, Vincristine, Taxol, Atorvastatin, Fluvastatin, Lovastatin, Pravastatin, Simvastatin, Terfenadine, Loratadine, Astemizole, Alfentanil, Carbamazepine, Azithromycin, Clarithromycin, Erythromycin, Itraconazole, Rifabutin, Lidocaine, Cisapride, Sertraline, Pimozide, Triazolam, Anastrazole, Busulfan, Corticosteroids (dexamethasone, methylprednisone and prednisone), Cytarabine, Docetaxel, Doxorubicin, Erlotinib, Exemestane, Gefitinib, Idarubicin, Ifosphamide, Imatinib mesylate, Irinotecan, Ketoconazole, Letrozole, Paclitaxel, Teniposide, Tretinoin, Vinorelbine, telithromycin: quinidine; alprazolam, diazepam, midazolam, nelfinavir, chlorpheniramine, amlodipine, diltiazem, lercanidipine, cerivastatin, estradiol, hydrocortisone, progesterone, testosterone, alfentanyl, aripiprazole, buspirone, cafergot, caffeine, cilostazol, cocaine, codeine, dapsone, dextromethorphan, docetaxel, domperidone, eplerenone, fentanyl, finasteride, gleevec, haloperidol, irinotecan, Levo-Alpha Acetyl Methadol (LAAM), methadone, nateglinide, odanestron, propranolol, quinine, salmetrol, sildenafil, terfenadine, trazodone, vincristine, zaleplon, zolpidem, ixabepilone, Agenerase (APV), Aptivus (TPV), Crixivan (IDV), Invirase (SQV), Lexiva (FPV), Prezista (DRV), Reyataz (ATV) Viracept (NFV), Elvitegravir, Selzentry, Vicriviroc, Telaprevir, Telithromycin, tandospirone, ibrutinib, canertinib, semaxinib, vatalanib, sorafenib, luflonamide, and buspirone.

20. A composition comprising a cytochrome inhibitor and trastuzumab emtansine.

21. The composition according to claim 20 wherein said cytochrome inhibitor is ritonavir, cobicistat, or structure (IA)

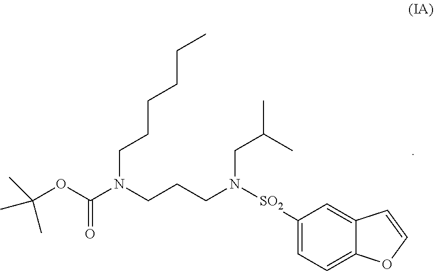 [SMILES: CCCCCCN(CCCN(CC(C)C)S(=O)(=O)c1ccc2occc2c1)C(=O)OC(C)(C)C]

22. (canceled)
